(19) United States
(12) Patent Application Publication
(10) Pub. No.: US 2019/0077795 A1
(43) Pub. Date: Mar. 14, 2019

(54) ARGININE METHYLTRANSFERASE INHIBITORS AND USES THEREOF

(71) Applicant: Epizyme, Inc., Cambridge, MA (US)

(72) Inventors: Lorna Helen Mitchell, Cambridge, MA (US); Kerren Kalai Swinger, Lexington, MA (US); Gideon Shapiro, Gainesville, FL (US); Paula Ann Boriack-Sjodin, Lexington, MA (US)

(73) Assignee: Epizyme, Inc., Cambridge, MA (US)

(21) Appl. No.: 16/101,176

(22) Filed: Aug. 10, 2018

Related U.S. Application Data
(63) Continuation of application No. 15/511,523, filed on Mar. 15, 2017, now abandoned.
(60) Provisional application No. 62/115,198, filed on Feb. 12, 2015.
(60) Provisional application No. 62/051,905, filed on Sep. 17, 2014.

(51) Int. Cl. C07D 405/12; A61P 37/00; C07D 403/12; A61P 21/00; A61P 3/00; A61P 25/00; A61P 9/00; C07D 231/12; C07D 413/04; C07D 405/04; C07D 403/10; C07D 403/04; C07D 401/04; C07D 405/14; C07D 401/14; A61P 35/00

(52) U.S. Cl. CPC C07D 405/12; A61P 37/00; A61P 35/00; A61P 21/00; A61P 3/00; A61P 25/00; A61P 9/00; C07D 231/12; C07D 413/04; C07D 405/04; C07D 403/10; C07D 403/04; C07D 401/04; C07D 405/14; C07D 401/14; C07D 403/12

(57) ABSTRACT

Provided herein are various compounds, and pharmaceutically acceptable salts thereof, and pharmaceutical compositions thereof, useful for inhibiting arginine methyltransferase activity. Methods of using the compounds for treating arginine methyltransferase-mediated disorders are also described.

### RELATED APPLICATIONS

The present application is a continuation of and claims priority under 35 U.S.C. § 120 to U.S. application, U.S. Ser. No. 15/511,523, filed Mar. 15, 2017, which is a national stage filing under 35 U.S.C. § 371 of international PCT application, PCT/US2015/050629, filed Sep. 17, 2015, which claims priority under 35 U.S.C. § 119(e) to U.S. provisional patent applications, U.S. Ser. No. 62/051,905, filed Sep. 17, 2014, and U.S. Ser. No. 62/115,198, filed Feb. 12, 2015, the entire contents of which are incorporated herein by reference.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., arginine methyltransferases), many of which are associated with specific genetic alterations that can cause human disease.

Disease-associated chromatin-modifying enzymes (e.g., arginine methyltransferases) play a role in diseases such as proliferative disorders, autoimmune disorders, muscular disorders, vascular disorders, metabolic disorders, and neurological disorders. Thus, there is a need for the development of small molecules that are capable of inhibiting the activity of arginine methyltransferases.

### Detailed Description of Certain Embodiments

Arginine methyltransferases are attractive targets for modulation given their role in the regulation of diverse biological processes. It has now been found that compounds described herein, and pharmaceutically acceptable salts and compositions thereof, are effective as inhibitors of arginine methyltransferases. Such compounds are listed in Table 1, infra.

In certain embodiments, compounds described herein inhibit activity of an arginine methyltransferase (RMT) (e.g., PRMT1, PRMT3, CARM1, PRMT6, and/or PRMT8). In certain embodiments, methods of inhibiting an arginine methyltransferase are provided which comprise contacting the arginine methyltransferase with an effective amount of a compound of Table 1, or a pharmaceutically acceptable salt thereof. The RMT may be purified or crude, and may be present in a cell, tissue, or a subject. Thus, such methods encompass inhibition of RMT activity both in vitro and in vivo. In certain embodiments, the RMT is wild-type. In certain embodiments, the RMT is overexpressed. In certain embodiments, the RMT is a mutant. In certain embodiments, the RMT is in a cell. In some embodiments, the RMT is expressed at normal levels in a subject, but the subject would benefit from RMT inhibition (e.g., because the subject has one or more mutations in an RMT substrate that causes an increase in methylation of the substrate with normal levels of RMT). In some embodiments, the RMT is in a subject known or identified as having abnormal RMT activity (e.g., overexpression).

In certain embodiments, methods of modulating gene expression in a cell are provided which comprise contacting a cell with an effective amount of a compound of Table 1, or a pharmaceutically acceptable salt thereof, or a pharmaceutical composition thereof. In certain embodiments, the cell in culture in vitro. In certain embodiments, cell is in an animal, e.g., a human.

In certain embodiments, methods of modulating transcription in a cell are provided which comprise contacting a cell with an effective amount of a compound of Table 1, or a pharmaceutically acceptable salt thereof, or a pharmaceutical composition thereof. In certain embodiments, the cell in culture in vitro. In certain embodiments, the cell is in an animal, e.g., a human.

In some embodiments, methods of treating an RMT-mediated disorder (e.g., a PRMT1-, PRMT3-, CARM1-, PRMT6-, or PRMT8-mediated disorder) are provided which comprise administering to a subject suffering from an RMT-mediated disorder an effective amount of a compound of Table 1, or a pharmaceutically acceptable salt thereof, or a pharmaceutical composition thereof. In certain embodiments, the RMT-mediated disorder is a proliferative disorder. In certain embodiments, compounds described herein are useful for treating cancer. In certain embodiments, compounds described herein are useful for treating breast cancer, prostate cancer, lung cancer, colon cancer, bladder cancer, or leukemia. In certain embodiments, the RMT-mediated disorder is a muscular disorder. In certain embodiments, the RMT-mediated disorder is an autoimmune disorder. In certain embodiments, the RMT-mediated disorder is a neurological disorder. In certain embodiments, the RMT-mediated disorder is a vascular disorder. In certain embodiments, the RMT-mediated disorder is a metabolic disorder.

Compounds described herein are also useful for the study of arginine methyltransferases in biological and pathological phenomena, the study of intracellular signal transduction pathways mediated by arginine methyltransferases, and the comparative evaluation of new RMT inhibitors.

This application refers to various issued patent, published patent applications, journal articles, and other publications, all of which are incorporated herein by reference.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

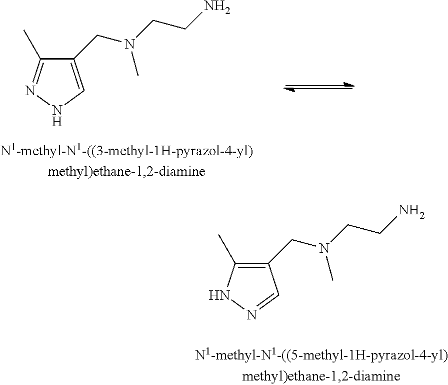

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, quaternary salts.

A “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), rodents (e.g., rats and/or mice), reptiles, amphibians, and fish. In certain embodiments, the non-human animal is a mammal. The non-human animal may be a male or female at any stage of development. A non-human animal may be a transgenic animal.

“Condition,” “disease,” and “disorder” are used interchangeably herein.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

An “effective amount” of a compound refers to an amount sufficient to elicit the desired biological response, e.g., treat the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “methyltransferase” represents transferase class enzymes that are able to transfer a methyl group from a donor molecule to an acceptor molecule, e.g., an amino acid residue of a protein or a nucleic base of a DNA molecule. Methytransferases typically use a reactive methyl group bound to sulfur in S-adenosyl methionine (SAM) as the methyl donor. In some embodiments, a methyltransferase described herein is a protein methyltransferase. In some embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and arginine residues of histone proteins. In certain embodiments, a methyltransferase described herein is a histone-arginine N-methyltransferase.

As generally described above, provided herein are compounds contemplated useful as arginine methyltransferase (RMT) inhibitors, i.e., compounds, or pharmaceutically acceptable salts thereof, as provided in Table 1:

| | | | PRMT1 | PRMT6 | PRMT8 | ICW |
|---|---|---|---|---|---|---|
| # | Structure | LCMS | IC₅₀ (uM) | IC₅₀ (uM) | IC₅₀ (uM) | EC₃₀ (uM) |
| 1 | 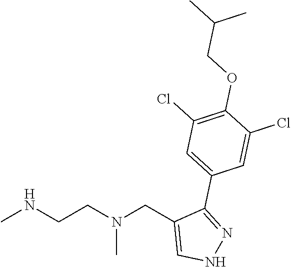 [SMILES: CNCCN(C)Cc1c[nH]nc1-c1cc(Cl)c(OCC(C)C)c(Cl)c1] | — | 0.00351 | 0.0104 | 0.01727 | 0.38 |
| 2 | 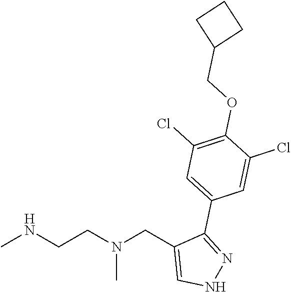 [SMILES: CNCCN(C)Cc1c[nH]nc1-c1cc(Cl)c(OCC2CCC2)c(Cl)c1] | — | 0.00361 | 0.01197 | 0.02176 | 0.304 |
| 3 | 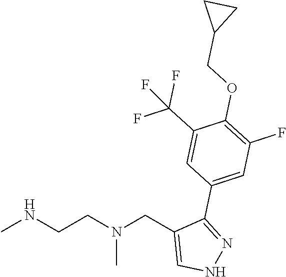 [SMILES: CNCCN(C)Cc1c[nH]nc1-c1cc(F)c(OCC2CC2)c(C(F)(F)F)c1] | — | 0.00398 | 0.0195 | 0.02187 | 0.664 |
| 5 | 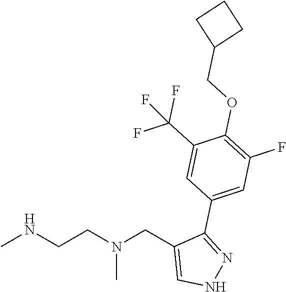 [SMILES: CNCCN(C)Cc1c[nH]nc1-c1cc(F)c(OCC2CCC2)c(C(F)(F)F)c1] | — | 0.00458 | 0.00685 | 0.01221 | 0.468 |
| 6 | 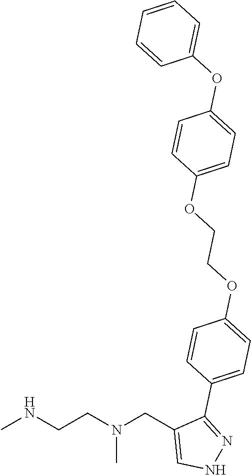 [SMILES: CNCCN(C)Cc1c[nH]nc1-c1ccc(OCCOc2ccc(Oc3ccccc3)cc2)cc1] | — | 0.00479 | 0.00343 | 0.01924 | 0.32824 |
| 7 | 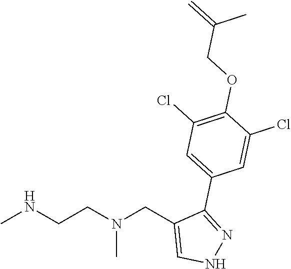 [SMILES: C=C(C)COc1c(Cl)cc(-c2n[nH]cc2CN(C)CCNC)cc1Cl] | — | 0.00486 | 0.01306 | 0.0124 | 0.09154 |
| 8 | 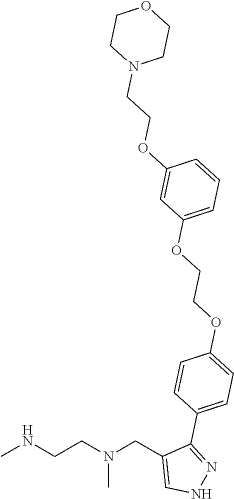 [SMILES: CNCCN(C)Cc1c[nH]nc1-c1ccc(OCCOc2cccc(OCCN3CCOCC3)c2)cc1] | — | 0.00491 | 0.00449 | 0.00939 | 0.2337 |
| 9 | 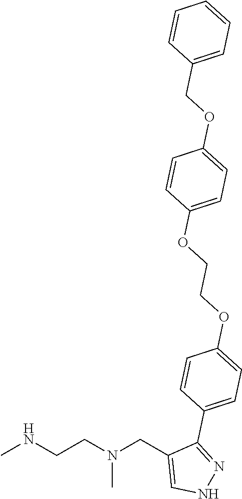 [SMILES: CNCCN(C)Cc1c[nH]nc1-c1ccc(OCCOc2ccc(OCc3ccccc3)cc2)cc1] | — | 0.00512 | 0.00222 | 0.02403 | 0.32912 |
| 10 | 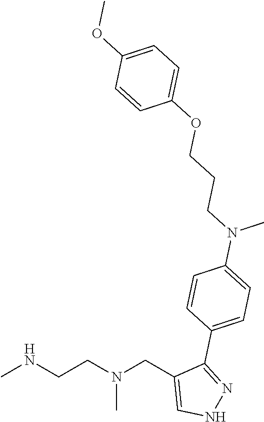 | — | 0.00521 | 0.00825 | 0.01242 | 0.1762 |
| 11 | 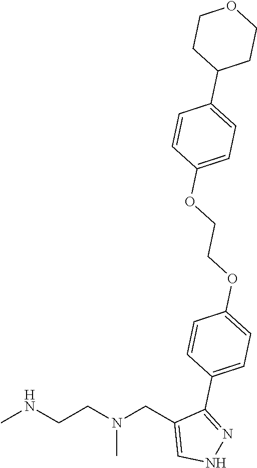 [SMILES: CNCCN(C)Cc1c[nH]nc1-c1ccc(OCCOc2ccc(C3CCOCC3)cc2)cc1] | — | 0.00523 | 0.00403 | 0.01333 | 0.11384 |
| 12 | 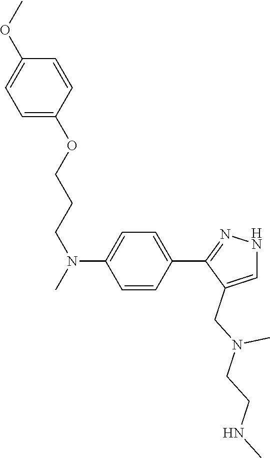 [SMILES: CNCCN(C)Cc1c[nH]nc1-c1ccc(N(C)CCCOc2ccc(OC)cc2)cc1] | — | 0.00525 | 0.01456 | 0.02643 | 0.44979 |
| 13 | 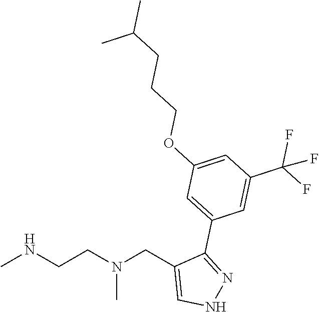 [SMILES: CNCCN(C)Cc1c[nH]nc1-c1cc(OCCCC(C)C)cc(C(F)(F)F)c1] | — | 0.00531 | 0.28535 | 0.02608 | — |
| 14 | 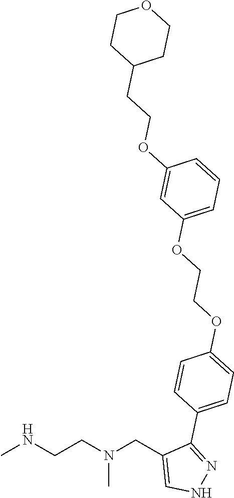 [SMILES: CNCCN(C)Cc1c[nH]nc1-c1ccc(OCCOc2cccc(OCCC3CCOCC3)c2)cc1] | — | 0.00549 | 0.00518 | 0.01461 | 0.3473 |
| 15 | 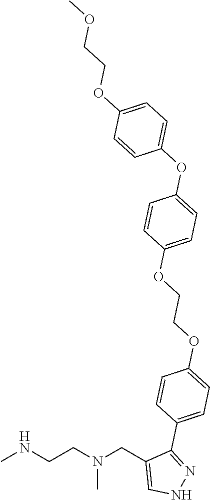 [SMILES: CNCCN(C)Cc1c[nH]nc1-c1ccc(OCCOc2ccc(Oc3ccc(OCCOC)cc3)cc2)cc1] | — | 0.00579 | 0.00238 | 0.01929 | 0.22006 |
| 16 | 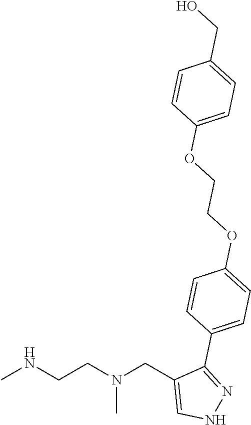 [SMILES: CNCCN(C)Cc1c[nH]nc1-c1ccc(OCCOc2ccc(CO)cc2)cc1] | — | 0.0058 | 0.0037 | 0.02058 | 0.27635 |
| 17 | 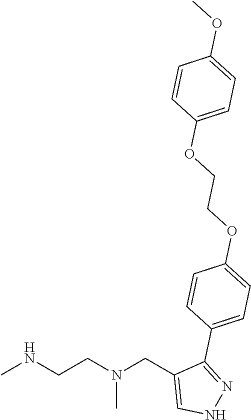 [SMILES: CNCCN(C)Cc1c[nH]nc1-c1ccc(OCCOc2ccc(OC)cc2)cc1] | — | 0.00585 | 0.00405 | 0.03359 | 0.086 |
| 18 | 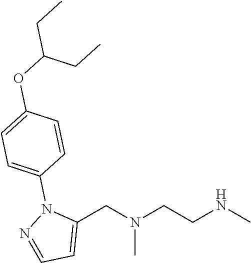 [SMILES: CCC(CC)Oc1ccc(-n2nccc2CN(C)CCNC)cc1] | — | 0.00586 | 0.06779 | 0.01533 | 0.09146 |
| 19 | 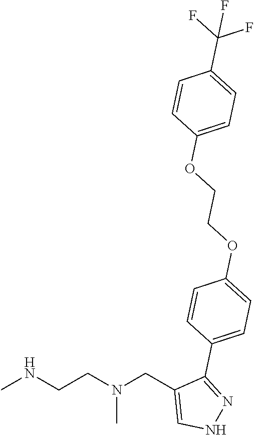 [SMILES: CNCCN(C)Cc1c[nH]nc1-c1ccc(OCCOc2ccc(C(F)(F)F)cc2)cc1] | — | 0.00608 | 0.00977 | 0.0366 | 0.32776 |
| 20 | 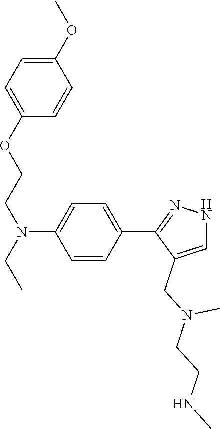 [SMILES: CCN(CCOc1ccc(OC)cc1)c1ccc(-c2n[nH]cc2CN(C)CCNC)cc1] | — | 0.00616 | 0.01691 | 0.01988 | 0.33622 |
| 21 | 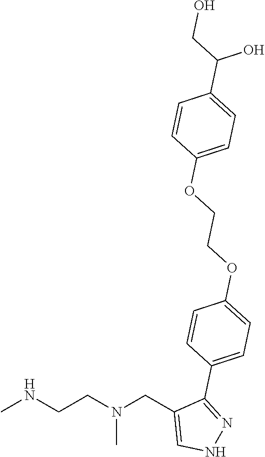 [SMILES: CNCCN(C)Cc1c[nH]nc1-c1ccc(OCCOc2ccc(C(O)CO)cc2)cc1] | — | 0.00623 | 0.00616 | 0.02597 | 3.18386 |
| 22 | 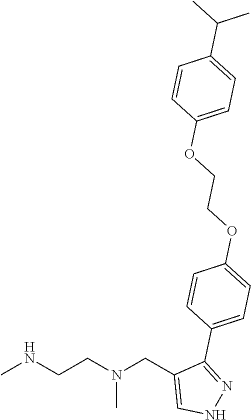 [SMILES: CNCCN(C)Cc1c[nH]nc1-c1ccc(OCCOc2ccc(C(C)C)cc2)cc1] | — | 0.00643 | 0.00574 | 0.03003 | 0.65458 |
| 23 | 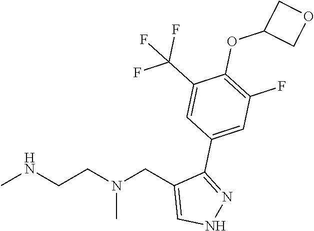 [SMILES: CNCCN(C)Cc1c[nH]nc1-c1cc(F)c(OC2COC2)c(C(F)(F)F)c1] | — | 0.00647 | 0.01688 | 0.06075 | 1.296 |
| 24 | 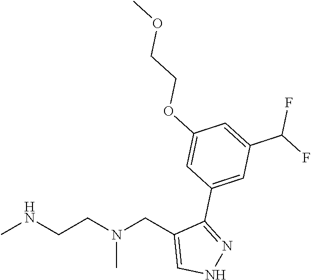 [SMILES: CNCCN(C)Cc1c[nH]nc1-c1cc(OCCOC)cc(C(F)F)c1] | — | 0.0065 | 0.21222 | 0.13209 | 1.251 |
| 25 | 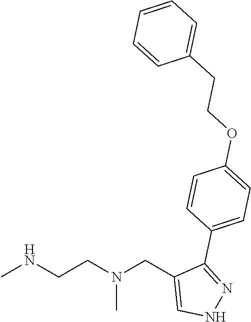 [SMILES: CNCCN(C)Cc1c[nH]nc1-c1ccc(OCCc2ccccc2)cc1] | — | 0.00668 | 0.01101 | 0.01734 | 0.097 |
| 26 | 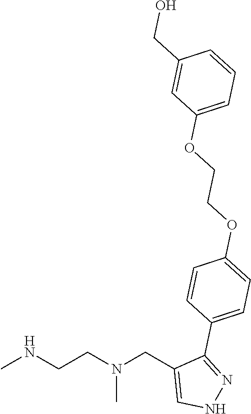 [SMILES: CNCCN(C)Cc1c[nH]nc1-c1ccc(OCCOc2cccc(CO)c2)cc1] | — | 0.00669 | 0.00448 | 0.02577 | 0.54428 |
| 27 | 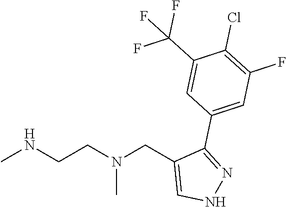 [SMILES: CNCCN(C)Cc1c[nH]nc1-c1cc(F)c(Cl)c(C(F)(F)F)c1] | — | 0.00675 | 0.0214 | 0.03698 | 0.377 |
| 28 | 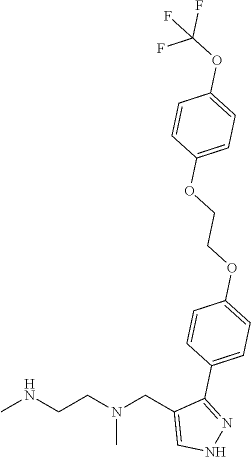 [SMILES: CNCCN(C)Cc1c[nH]nc1-c1ccc(OCCOc2ccc(OC(F)(F)F)cc2)cc1] | — | 0.00683 | 0.00703 | 0.05262 | 0.47 |
| 29 | 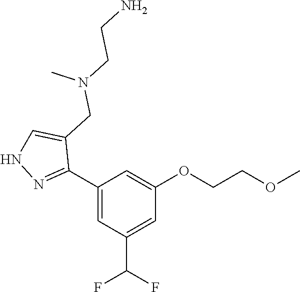 [SMILES: COCCOc1cc(-c2n[nH]cc2CN(C)CCN)cc(C(F)F)c1] | — | 0.00694 | 0.27593 | 0.11214 | 5.437 |
| 30 | 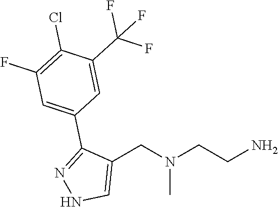 [SMILES: CN(CCN)Cc1c[nH]nc1-c1cc(F)c(Cl)c(C(F)(F)F)c1] | — | 0.00705 | 0.01353 | 0.06081 | 0.648 |
| 31 | 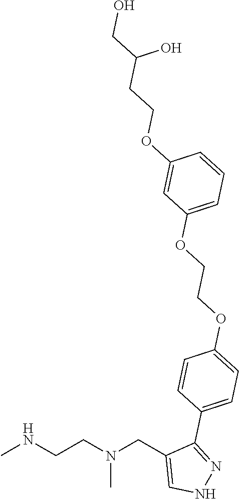 [SMILES: CNCCN(C)Cc1c[nH]nc1-c1ccc(OCCOc2cccc(OCCC(O)CO)c2)cc1] | — | 0.00708 | 0.00432 | 0.02002 | 2.3254 |
| 32 | 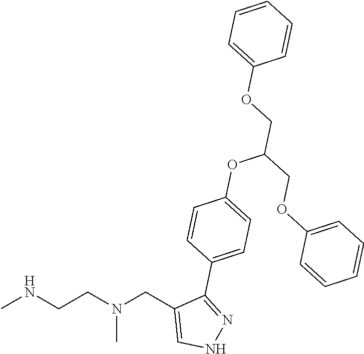 [SMILES: CNCCN(C)Cc1c[nH]nc1-c1ccc(OC(COc2ccccc2)COc2ccccc2)cc1] | — | 0.00732 | 0.00624 | 0.03747 | 1.40277 |
| 33 | 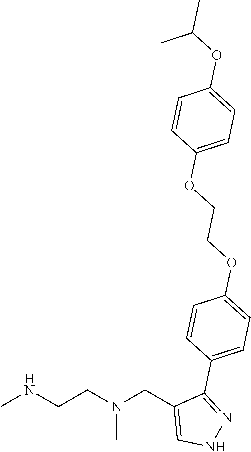 [SMILES: CNCCN(C)Cc1c[nH]nc1-c1ccc(OCCOc2ccc(OC(C)C)cc2)cc1] | — | 0.00737 | 0.00562 | 0.02333 | 0.232 |
| 34 | 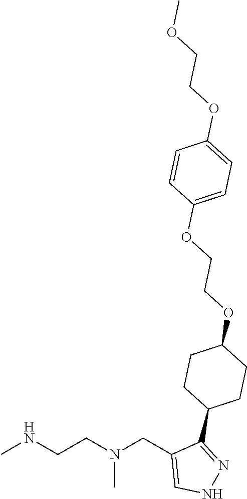 [SMILES: CNCCN(C)Cc1c[nH]nc1[C@H]1CC[C@@H](OCCOc2ccc(OCCOC)cc2)CC1] | — | 0.00737 | 0.0267 | 0.01354 | 0.08013 |
| 35 | 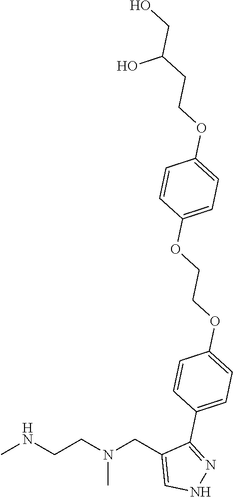 [SMILES: CNCCN(C)Cc1c[nH]nc1-c1ccc(OCCOc2ccc(OCCC(O)CO)cc2)cc1] | — | 0.00759 | 0.005 | 0.02187 | 0.42273 |
| 36 | 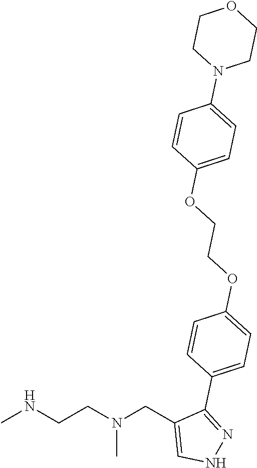 [SMILES: CNCCN(C)Cc1c[nH]nc1-c1ccc(OCCOc2ccc(N3CCOCC3)cc2)cc1] | — | 0.00764 | 0.00635 | 0.02435 | 0.1271 |
| 37 | 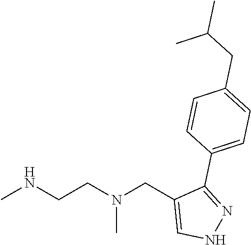 [SMILES: CNCCN(C)Cc1c[nH]nc1-c1ccc(CC(C)C)cc1] | — | 0.00804 | 0.01688 | 0.01364 | 0.082 |
| 38 | 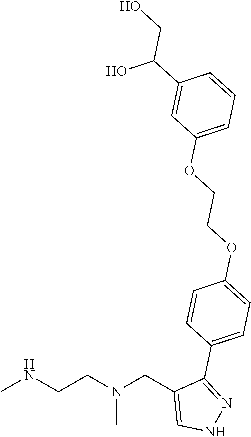 [SMILES: CNCCN(C)Cc1c[nH]nc1-c1ccc(OCCOc2cccc(C(O)CO)c2)cc1] | — | 0.00822 | 0.00632 | 0.03982 | 8.68816 |
| 39 | 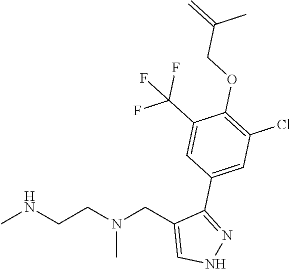 [SMILES: C=C(C)COc1c(Cl)cc(-c2n[nH]cc2CN(C)CCNC)cc1C(F)(F)F] | — | 0.00831 | 0.02949 | 0.01219 | 0.439 |
| 40 | 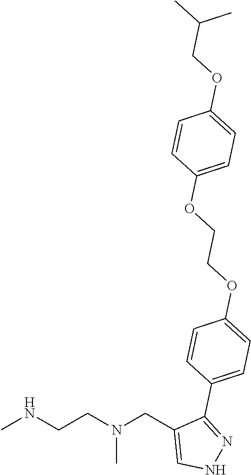 [SMILES: CNCCN(C)Cc1c[nH]nc1-c1ccc(OCCOc2ccc(OCC(C)C)cc2)cc1] | — | 0.00834 | 0.00739 | 0.02466 | 0.5051 |
| 41 | 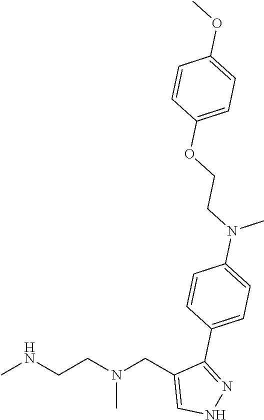 | — | 0.0088 | 0.00896 | 0.03338 | 0.35138 |
| 42 | 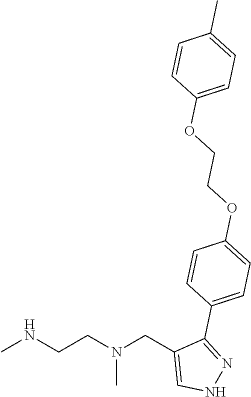 [SMILES: CNCCN(C)Cc1c[nH]nc1-c1ccc(OCCOc2ccc(C)cc2)cc1] | — | 0.0089 | 0.00498 | 0.05139 | 0.185 |
| 43 | 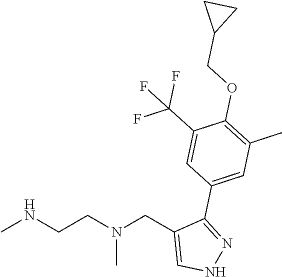 | — | 0.00897 | 0.03262 | 0.01505 | 0.475 |
| 44 | 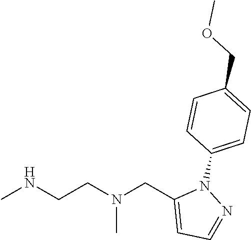 | — | 0.00925 | 0.05645 | 0.02355 | 0.12999 |
| 45 | 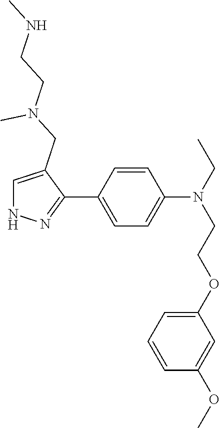 [SMILES: CCN(CCOc1cccc(OC)c1)c1ccc(-c2n[nH]cc2CN(C)CCNC)cc1] | — | 0.00926 | 0.03311 | 0.03672 | 1.13966 |
| 46 | 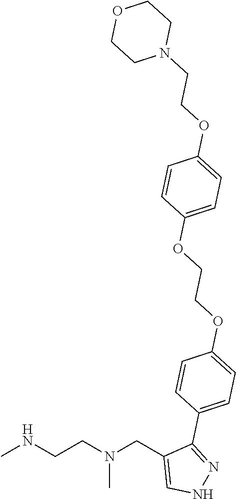 [SMILES: CNCCN(C)Cc1c[nH]nc1-c1ccc(OCCOc2ccc(OCCN3CCOCC3)cc2)cc1] | — | 0.00949 | 0.00456 | 0.02603 | 0.25157 |
| 48 | 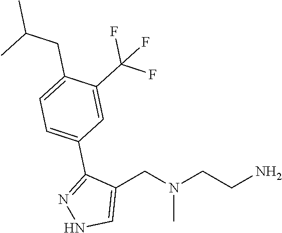 [SMILES: CC(C)Cc1ccc(-c2n[nH]cc2CN(C)CCN)cc1C(F)(F)F] | — | 0.01042 | 0.04267 | 0.0237 | 3.673 |
| 49 | 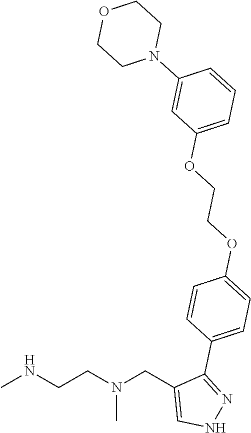 [SMILES: CNCCN(C)Cc1c[nH]nc1-c1ccc(OCCOc2cccc(N3CCOCC3)c2)cc1] | — | 0.0105 | 0.00705 | 0.03369 | 0.74682 |
| 50 | 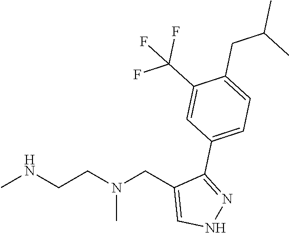 [SMILES: CNCCN(C)Cc1c[nH]nc1-c1ccc(CC(C)C)c(C(F)(F)F)c1] | — | 0.01054 | 0.0677 | 0.02453 | — |
| 52 | 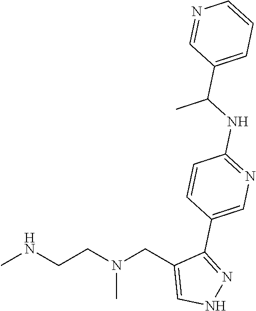 [SMILES: CNCCN(C)Cc1c[nH]nc1-c1ccc(NC(C)c2cccnc2)nc1] | — | 0.01073 | 0.0206 | 0.02601 | — |
| 53 | 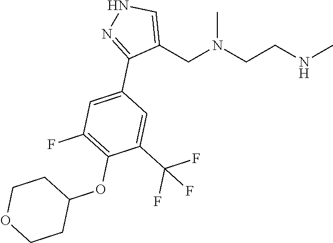 [SMILES: CNCCN(C)Cc1c[nH]nc1-c1cc(F)c(OC2CCOCC2)c(C(F)(F)F)c1] | — | 0.01097 | 0.01887 | 0.03285 | 0.479 |
| 54 | 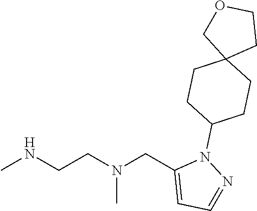 [SMILES: CNCCN(C)Cc1ccnn1C1CCC2(CCOC2)CC1] | — | 0.01118 | 0.03293 | 0.05354 | 0.68 |
| 55 | 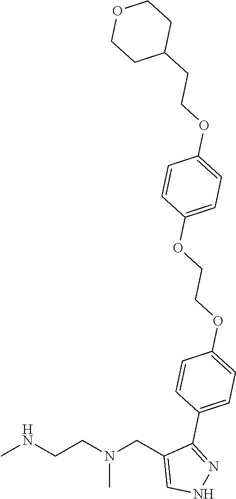 [SMILES: CNCCN(C)Cc1c[nH]nc1-c1ccc(OCCOc2ccc(OCCC3CCOCC3)cc2)cc1] | — | 0.01131 | 0.01863 | 0.02297 | — |
| 56 | 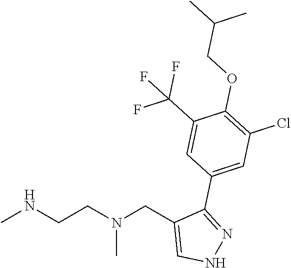 [SMILES: CNCCN(C)Cc1c[nH]nc1-c1cc(Cl)c(OCC(C)C)c(C(F)(F)F)c1] | — | 0.01153 | 0.04355 | 0.01683 | 0.765 |
| 57 | 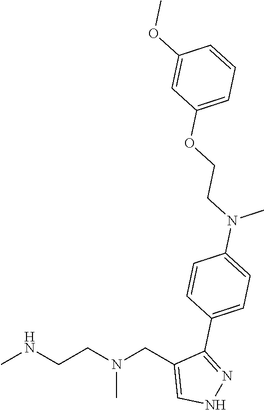 | — | 0.01182 | 0.00902 | 0.04201 | 0.75766 |
| 58 | 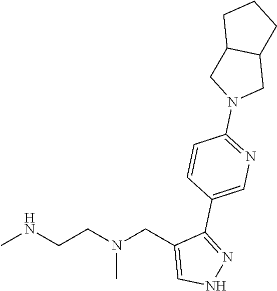 [SMILES: CNCCN(C)Cc1c[nH]nc1-c1ccc(N2CC3CCCC3C2)nc1] | — | 0.01189 | 0.00835 | 0.02544 | 0.4165 |
| 59 | 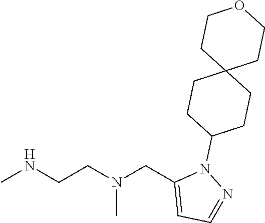 [SMILES: CNCCN(C)Cc1ccnn1C1CCC2(CCOCC2)CC1] | — | 0.01217 | 0.03472 | 0.04201 | 0.24637 |
| 60 | 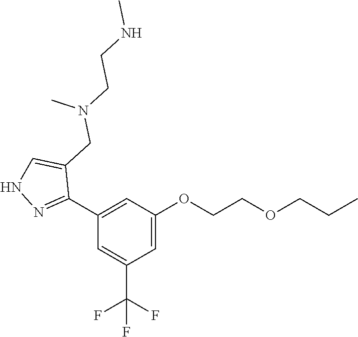 [SMILES: CCCOCCOc1cc(-c2n[nH]cc2CN(C)CCNC)cc(C(F)(F)F)c1] | — | 0.01271 | 1.19723 | 0.0525 | 1.263 |
| 61 | 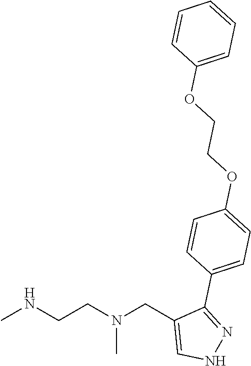 [SMILES: CNCCN(C)Cc1c[nH]nc1-c1ccc(OCCOc2ccccc2)cc1] | — | 0.01286 | 0.01136 | 0.0374 | 0.13 |
| 62 | 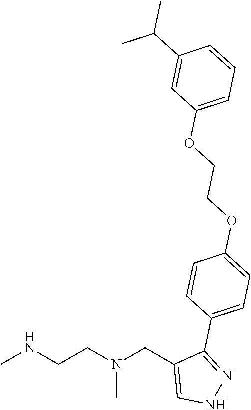 [SMILES: CNCCN(C)Cc1c[nH]nc1-c1ccc(OCCOc2cccc(C(C)C)c2)cc1] | — | 0.01294 | 0.00431 | 0.0363 | 1.37832 |
| 63 | 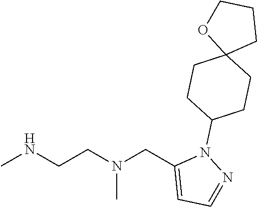 [SMILES: CNCCN(C)Cc1ccnn1C1CCC2(CCCO2)CC1] | — | 0.0131 | 0.03173 | 0.04448 | 0.17274 |
| 64 | 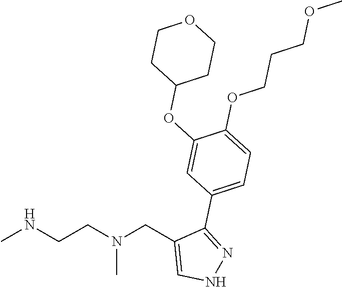 | — | 0.01391 | 0.05354 | 0.24863 | 4.628 |
| 65 | 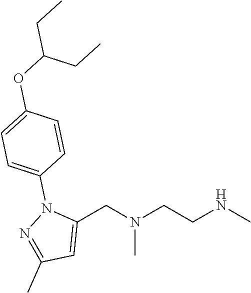 [SMILES: CCC(CC)Oc1ccc(-n2nc(C)cc2CN(C)CCNC)cc1] | — | 0.01409 | 0.13561 | 0.02994 | 0.21559 |
| 66 | 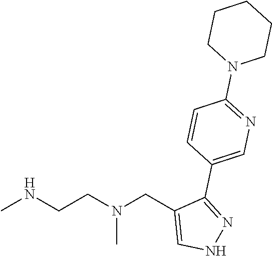 [SMILES: CNCCN(C)Cc1c[nH]nc1-c1ccc(N2CCCCC2)nc1] | — | 0.01422 | 0.04075 | 0.03706 | 0.39311 |
| 67 | 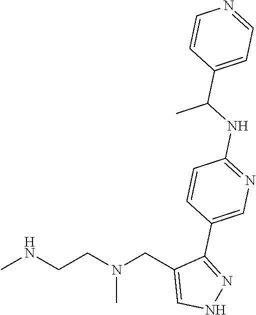 [SMILES: CNCCN(C)Cc1c[nH]nc1-c1ccc(NC(C)c2ccncc2)nc1] | — | 0.01469 | 0.03096 | 0.06198 | — |
| 68 | 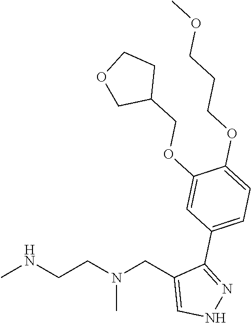 [SMILES: CNCCN(C)Cc1c[nH]nc1-c1ccc(OCCCOC)c(OCC2CCOC2)c1] | — | 0.01478 | 0.03804 | 0.29548 | 3.454 |
| 69 | 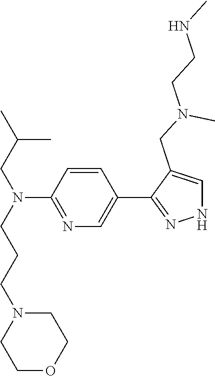 [SMILES: CNCCN(C)Cc1c[nH]nc1-c1ccc(N(CCCN2CCOCC2)CC(C)C)nc1] | — | 0.01494 | 2.5416 | 0.03119 | 1.33651 |
| 70 | 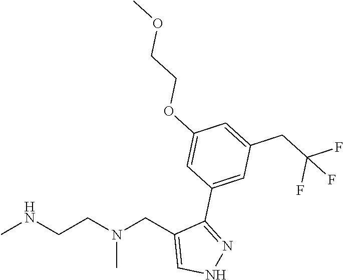 [SMILES: CNCCN(C)Cc1c[nH]nc1-c1cc(CC(F)(F)F)cc(OCCOC)c1] | — | 0.01523 | 1.26976 | 0.05114 | 0.49401 |
| 71 | 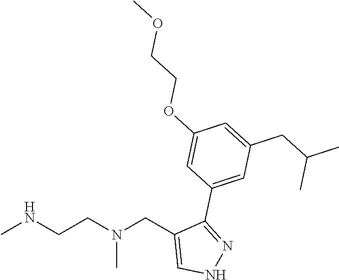 [SMILES: CNCCN(C)Cc1c[nH]nc1-c1cc(CC(C)C)cc(OCCOC)c1] | — | 0.01526 | 4.38951 | 0.04524 | 0.68 |
| 72 | 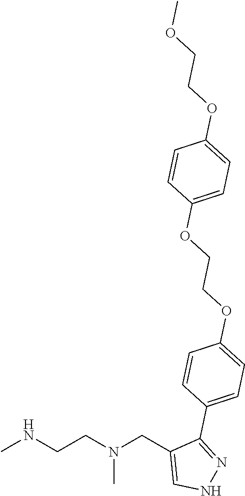 [SMILES: CNCCN(C)Cc1c[nH]nc1-c1ccc(OCCOc2ccc(OCCOC)cc2)cc1] | — | 0.01533 | 0.0101 | 0.03504 | 0.163 |
| 73 | 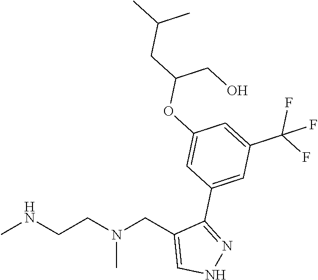 [SMILES: CNCCN(C)Cc1c[nH]nc1-c1cc(OC(CO)CC(C)C)cc(C(F)(F)F)c1] | — | 0.01605 | 1.90446 | 0.04384 | 2.102 |
| 74 | 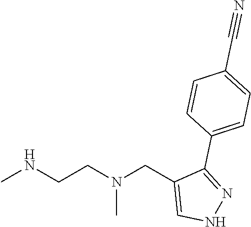 [SMILES: CNCCN(C)Cc1c[nH]nc1-c1ccc(C#N)cc1] | — | 0.01614 | 0.02705 | 0.02312 | 0.541 |
| 75 | 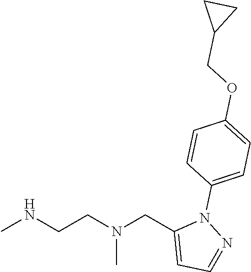 [SMILES: CNCCN(C)Cc1ccnn1-c1ccc(OCC2CC2)cc1] | — | 0.01617 | 0.04947 | 0.04869 | 0.53806 |
| 76 | 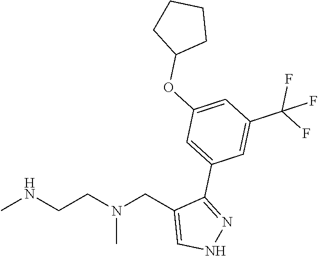 [SMILES: CNCCN(C)Cc1c[nH]nc1-c1cc(OC2CCCC2)cc(C(F)(F)F)c1] | — | 0.0163 | 0.25397 | 0.08713 | — |
| 78 | 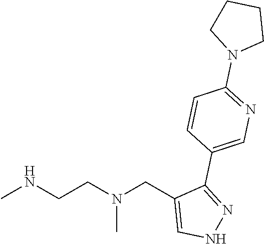 [SMILES: CNCCN(C)Cc1c[nH]nc1-c1ccc(N2CCCC2)nc1] | — | 0.01828 | 0.0395 | 0.03094 | 0.75705 |
| 79 | 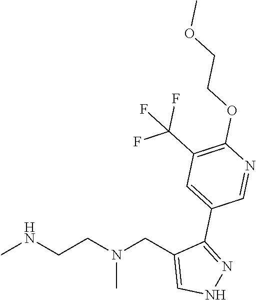 [SMILES: CNCCN(C)Cc1c[nH]nc1-c1cnc(OCCOC)c(C(F)(F)F)c1] | — | 0.01978 | 0.10506 | 0.17134 | 2.611 |
| 80 | 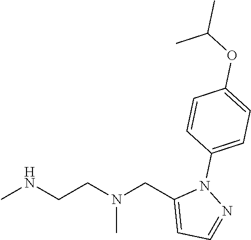 [SMILES: CNCCN(C)Cc1ccnn1-c1ccc(OC(C)C)cc1] | — | 0.01984 | 0.11406 | 0.04935 | 0.411 |
| 81 | 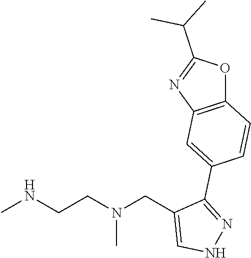 [SMILES: CNCCN(C)Cc1c[nH]nc1-c1ccc2oc(C(C)C)nc2c1] | — | 0.0199 | 0.04446 | 0.09471 | 0.927 |
| 82 | 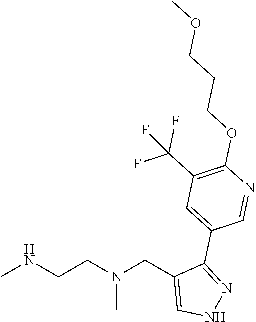 [SMILES: CNCCN(C)Cc1c[nH]nc1-c1cnc(OCCCOC)c(C(F)(F)F)c1] | — | 0.0203 | 0.07774 | 0.18873 | 4.06 |
| 83 | 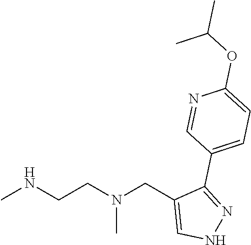 [SMILES: CNCCN(C)Cc1c[nH]nc1-c1ccc(OC(C)C)nc1] | — | 0.02053 | 0.06902 | 0.03365 | 0.2 |
| 84 | 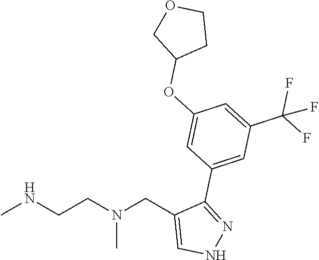 [SMILES: CNCCN(C)Cc1c[nH]nc1-c1cc(OC2CCOC2)cc(C(F)(F)F)c1] | — | 0.02069 | 0.70669 | 0.22761 | 4.957 |
| 85 | 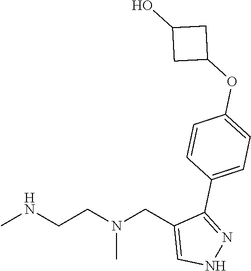 [SMILES: CNCCN(C)Cc1c[nH]nc1-c1ccc(OC2CC(O)C2)cc1] | — | 0.02134 | 0.01436 | 0.08526 | 3.594 |
| 86 | 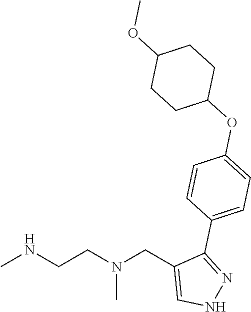 [SMILES: CNCCN(C)Cc1c[nH]nc1-c1ccc(OC2CCC(OC)CC2)cc1] | — | 0.0225 | 0.01962 | 0.06855 | 0.836 |
| 87 | 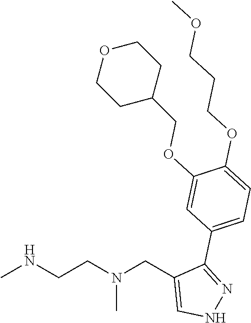 [SMILES: CNCCN(C)Cc1c[nH]nc1-c1ccc(OCCCOC)c(OCC2CCOCC2)c1] | — | 0.02266 | 0.0783 | 0.42408 | 6.199 |
| 88 | 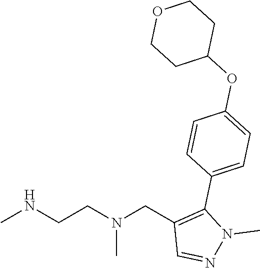 | — | 0.02301 | 0.02355 | 0.02667 | 1.236 |
| 89 | 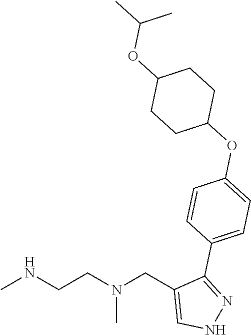 [SMILES: CNCCN(C)Cc1c[nH]nc1-c1ccc(OC2CCC(OC(C)C)CC2)cc1] | — | 0.02311 | 0.02513 | 0.04751 | 0.44905 |
| 90 | 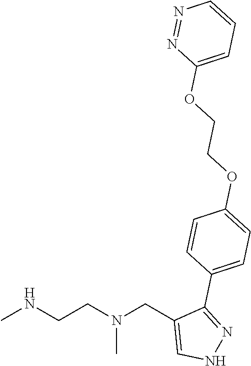 [SMILES: CNCCN(C)Cc1c[nH]nc1-c1ccc(OCCOc2cccnn2)cc1] | — | 0.02425 | 0.01988 | 0.11703 | 2.49103 |
| 91 | 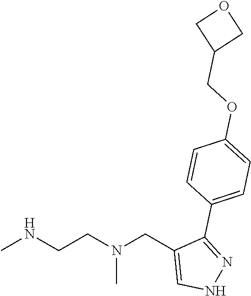 [SMILES: CNCCN(C)Cc1c[nH]nc1-c1ccc(OCC2COC2)cc1] | — | 0.02467 | 0.03221 | 0.28235 | 3.058 |
| 92 | 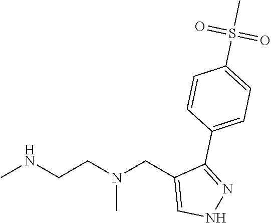 [SMILES: CNCCN(C)Cc1c[nH]nc1-c1ccc(S(C)(=O)=O)cc1] | — | 0.02474 | 0.03086 | 0.043 | 2.126 |
| 93 | 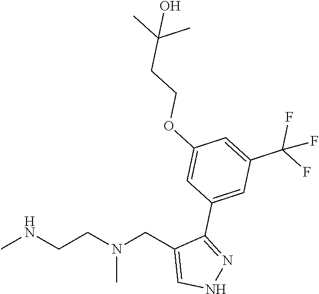 [SMILES: CNCCN(C)Cc1c[nH]nc1-c1cc(OCCC(C)(C)O)cc(C(F)(F)F)c1] | — | 0.02476 | 3.38417 | 0.32644 | 4.55 |
| 94 | 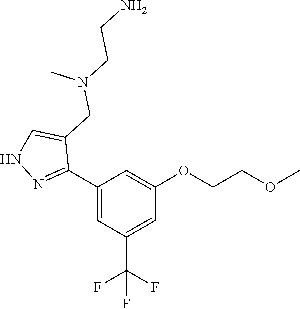 [SMILES: COCCOc1cc(-c2n[nH]cc2CN(C)CCN)cc(C(F)(F)F)c1] | — | 0.02549 | 0.82305 | 0.15791 | 6.823 |
| 95 | 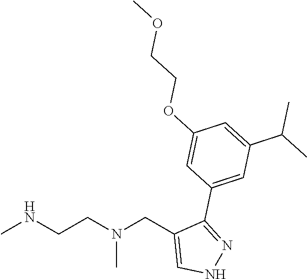 [SMILES: CNCCN(C)Cc1c[nH]nc1-c1cc(OCCOC)cc(C(C)C)c1] | — | 0.02584 | 0.77135 | 0.08435 | 0.726 |
| 96 | 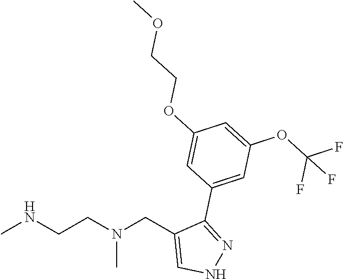 [SMILES: CNCCN(C)Cc1c[nH]nc1-c1cc(OCCOC)cc(OC(F)(F)F)c1] | — | 0.0267 | 5.12777 | 0.10797 | 1.762 |
| 97 | 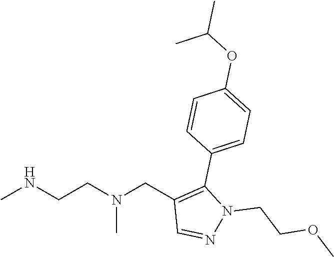 | — | 0.02821 | 0.10643 | 0.06011 | 1.994 |
| 98 | 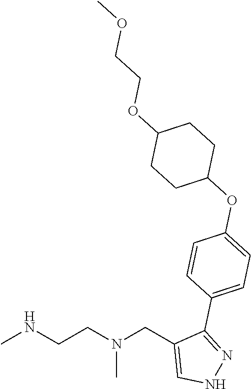 [SMILES: CNCCN(C)Cc1c[nH]nc1-c1ccc(OC2CCC(OCCOC)CC2)cc1] | — | 0.02849 | 0.02885 | 0.11308 | 0.86852 |
| 99 | 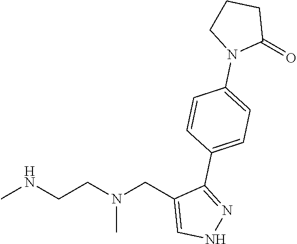 [SMILES: CNCCN(C)Cc1c[nH]nc1-c1ccc(N2CCCC2=O)cc1] | — | 0.0297 | 0.06328 | 0.12701 | 3.944 |
| 100 | 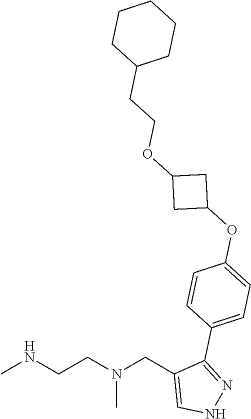 [SMILES: CNCCN(C)Cc1c[nH]nc1-c1ccc(OC2CC(OCCC3CCCCC3)C2)cc1] | — | 0.02996 | 0.00465 | 0.05986 | — |
| 101 | 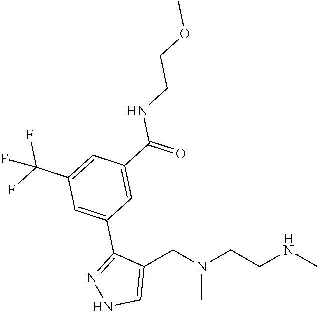 [SMILES: CNCCN(C)Cc1c[nH]nc1-c1cc(C(=O)NCCOC)cc(C(F)(F)F)c1] | — | 0.03088 | 2.65528 | 0.3423 | 11.123 |
| 102 | 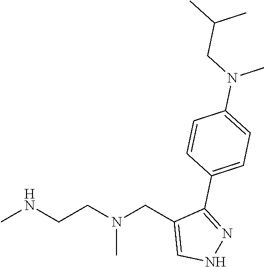 | — | 0.03193 | 0.23642 | 0.08185 | 1.057 |
| 103 | 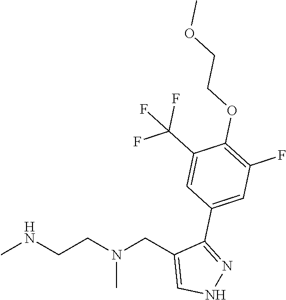 [SMILES: CNCCN(C)Cc1c[nH]nc1-c1cc(F)c(OCCOC)c(C(F)(F)F)c1] | — | 0.03327 | 0.02745 | 0.04209 | 0.85 |
| 104 | 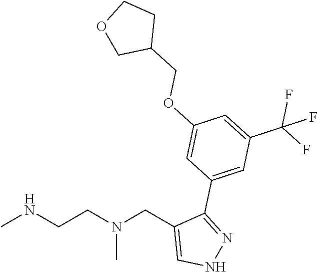 [SMILES: CNCCN(C)Cc1c[nH]nc1-c1cc(OCC2CCOC2)cc(C(F)(F)F)c1] | — | 0.03329 | 2.90341 | 0.22634 | 1.467 |
| 105 | 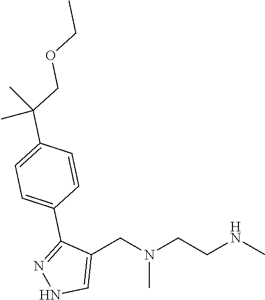 [SMILES: CCOCC(C)(C)c1ccc(-c2n[nH]cc2CN(C)CCNC)cc1] | — | 0.03396 | 0.05586 | 0.10625 | 2.748 |
| 106 | 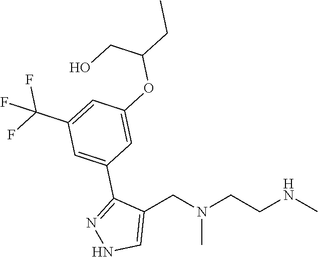 [SMILES: CCC(CO)Oc1cc(-c2n[nH]cc2CN(C)CCNC)cc(C(F)(F)F)c1] | — | 0.03405 | 3.50701 | 0.20133 | 3.7 |
| 107 | 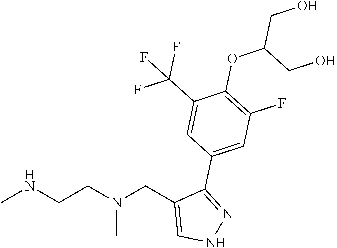 [SMILES: CNCCN(C)Cc1c[nH]nc1-c1cc(F)c(OC(CO)CO)c(C(F)(F)F)c1] | — | 0.03555 | 0.066 | 0.08498 | — |
| 108 | 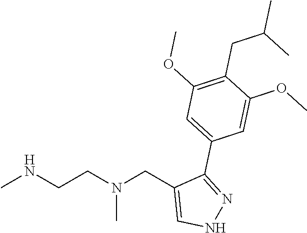 | — | 0.0357 | 0.82411 | 0.07119 | 2.967 |
| 109 | 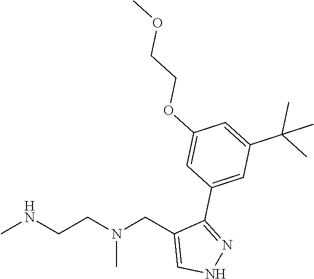 [SMILES: CNCCN(C)Cc1c[nH]nc1-c1cc(OCCOC)cc(C(C)(C)C)c1] | — | 0.03606 | 7.11945 | 0.20331 | 1.928 |
| 110 | 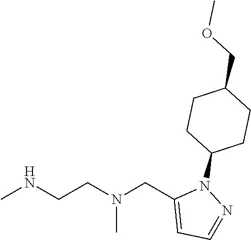 [SMILES: CNCCN(C)Cc1ccnn1[C@H]1CC[C@@H](COC)CC1] | — | 0.0364 | 0.14271 | — | 1.20412 |
| 111 | 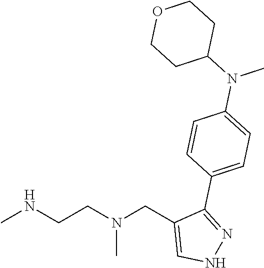 | — | 0.03673 | 0.95539 | 0.25208 | 1.04 |
| 112 | 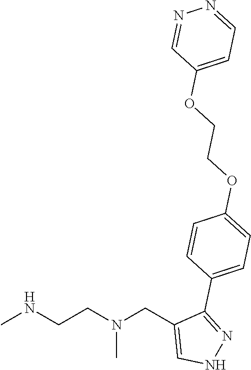 [SMILES: CNCCN(C)Cc1c[nH]nc1-c1ccc(OCCOc2ccnnc2)cc1] | — | 0.03733 | 0.02784 | — | 3.994 |
| 113 | 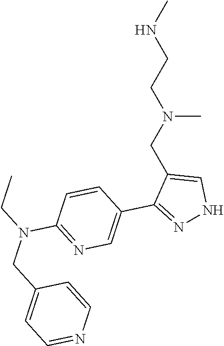 [SMILES: CCN(Cc1ccncc1)c1ccc(-c2n[nH]cc2CN(C)CCNC)cn1] | — | 0.03735 | 0.10456 | 0.24255 | — |
| 114 | 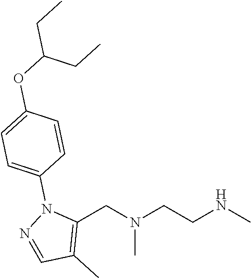 [SMILES: CCC(CC)Oc1ccc(-n2ncc(C)c2CN(C)CCNC)cc1] | — | 0.03806 | 0.58986 | 0.08359 | 1.85487 |
| 115 | 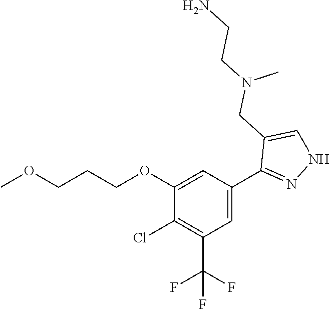 [SMILES: COCCCOc1cc(-c2n[nH]cc2CN(C)CCN)cc(C(F)(F)F)c1Cl] | — | 0.03906 | 1.82082 | 0.56294 | — |
| 116 | 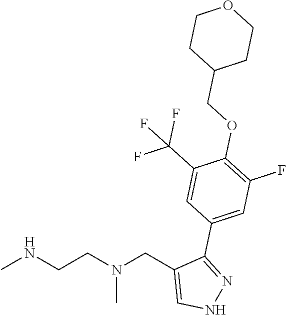 [SMILES: CNCCN(C)Cc1c[nH]nc1-c1cc(F)c(OCC2CCOCC2)c(C(F)(F)F)c1] | — | 0.0391 | 0.01837 | 0.11813 | 3.806 |
| 117 | 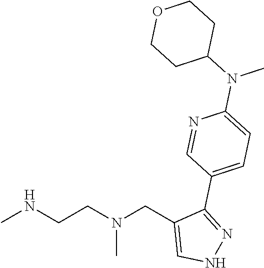 | — | 0.03921 | 2.31569 | 0.31739 | 8.433 |
| 118 | 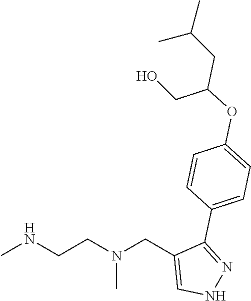 [SMILES: CNCCN(C)Cc1c[nH]nc1-c1ccc(OC(CO)CC(C)C)cc1] | — | 0.04116 | 0.03423 | 0.0955 | 1.175 |
| 119 | 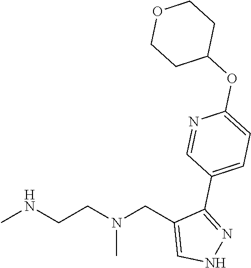 [SMILES: CNCCN(C)Cc1c[nH]nc1-c1ccc(OC2CCOCC2)nc1] | — | 0.04155 | 0.03292 | 0.12343 | 1.292 |
| 120 | 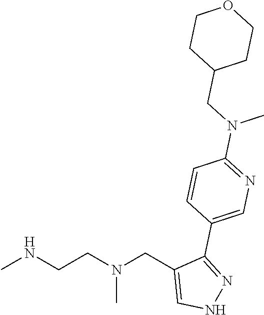 | — | 0.0417 | 0.27207 | — | 4.389 |
| 121 | 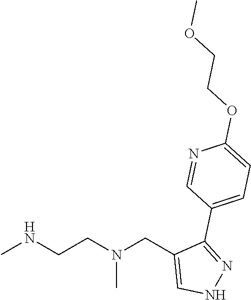 [SMILES: CNCCN(C)Cc1c[nH]nc1-c1ccc(OCCOC)nc1] | — | 0.04231 | 0.08861 | 0.16285 | 1.472 |
| 122 | 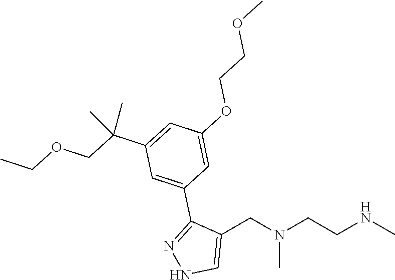 [SMILES: CCOCC(C)(C)c1cc(OCCOC)cc(-c2n[nH]cc2CN(C)CCNC)c1] | — | 0.04298 | >10.0 uM | 0.37053 | 6.626 |
| 123 | 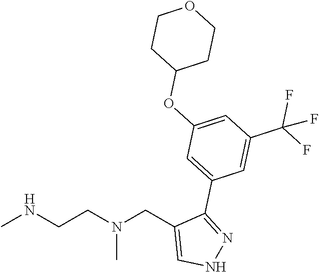 [SMILES: CNCCN(C)Cc1c[nH]nc1-c1cc(OC2CCOCC2)cc(C(F)(F)F)c1] | — | 0.04388 | 1.46842 | 0.37627 | 18.364 |
| 124 | 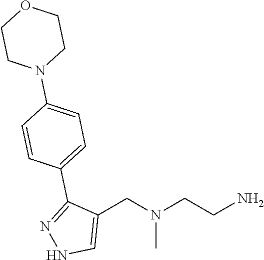 [SMILES: CN(CCN)Cc1c[nH]nc1-c1ccc(N2CCOCC2)cc1] | — | 0.04428 | 0.10709 | 0.41267 | 3.978 |
| 125 | 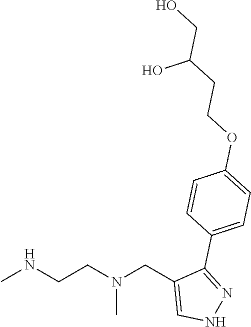 [SMILES: CNCCN(C)Cc1c[nH]nc1-c1ccc(OCCC(O)CO)cc1] | — | 0.0455 | 0.05477 | 0.26866 | 10.45 |
| 126 | 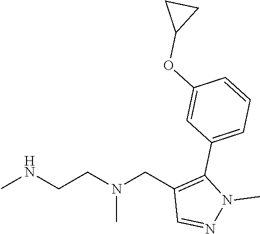 | — | 0.04553 | 0.0737 | 0.04458 | 1.272 |
| 127 | 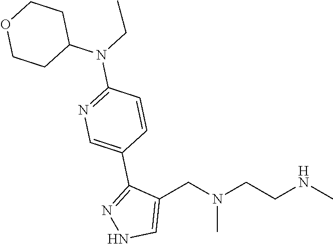 [SMILES: CCN(c1ccc(-c2n[nH]cc2CN(C)CCNC)cn1)C1CCOCC1] | — | 0.04585 | 3.447 | 0.46651 | 9.19 |
| 128 | 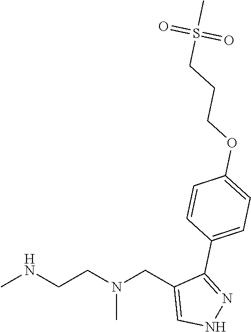 [SMILES: CNCCN(C)Cc1c[nH]nc1-c1ccc(OCCCS(C)(=O)=O)cc1] | — | 0.04642 | 0.03427 | 0.13144 | 13.334 |
| 129 | 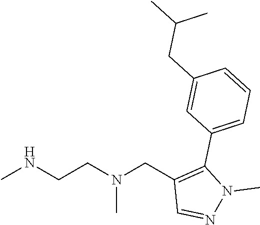 | — | 0.04754 | 0.21913 | 0.08765 | 7.415 |
| 130 | 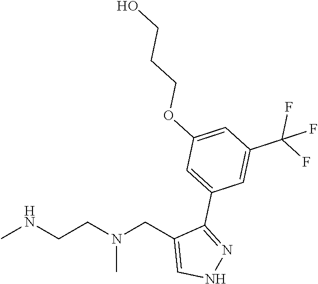 [SMILES: CNCCN(C)Cc1c[nH]nc1-c1cc(OCCCO)cc(C(F)(F)F)c1] | — | 0.04886 | 0.61899 | 0.29993 | 11.844 |
| 131 | 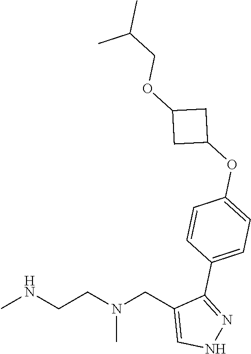 [SMILES: CNCCN(C)Cc1c[nH]nc1-c1ccc(OC2CC(OCC(C)C)C2)cc1] | — | 0.04924 | 0.01819 | — | 14.756 |
| 132 | 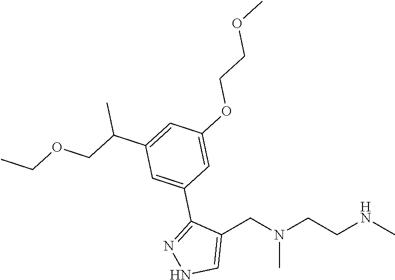 [SMILES: CCOCC(C)c1cc(OCCOC)cc(-c2n[nH]cc2CN(C)CCNC)c1] | — | 0.04942 | >10.0 | 0.14986 | 3.608 |
| 133 | 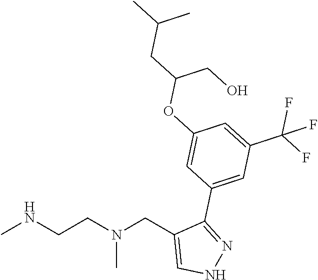 [SMILES: CNCCN(C)Cc1c[nH]nc1-c1cc(OC(CO)CC(C)C)cc(C(F)(F)F)c1] | 429.1 | 0.01605 | 1.90446 | 0.04384 | 2.102 |
| 134 | 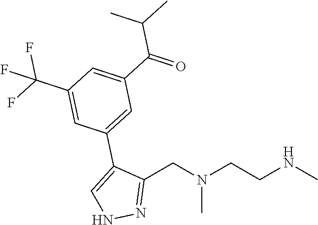 [SMILES: CNCCN(C)Cc1n[nH]cc1-c1cc(C(=O)C(C)C)cc(C(F)(F)F)c1] | 383.35 | 0.12138 | 2.06728 | 0.16787 | >6.7 |
| 135 | 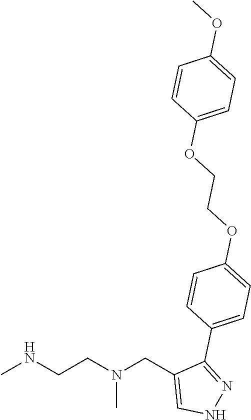 [SMILES: CNCCN(C)Cc1c[nH]nc1-c1ccc(OCCOc2ccc(OC)cc2)cc1] | 411.25 | 0.00585 | 0.00405 | 0.03359 | 0.086 |
| 136 | 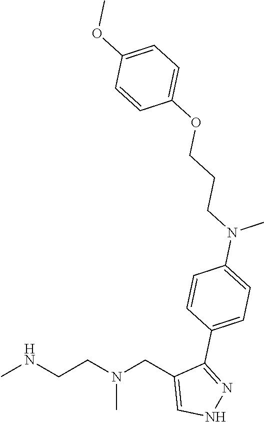 | 438.3 | 0.00521 | 0.00825 | 0.01242 | 0.1762 |
| 137 | 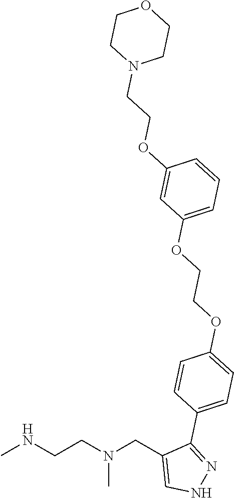 [SMILES: CNCCN(C)Cc1c[nH]nc1-c1ccc(OCCOc2cccc(OCCN3CCOCC3)c2)cc1] | 510.2 | 0.00491 | 0.00449 | 0.00939 | 0.2337 |
| 138 | 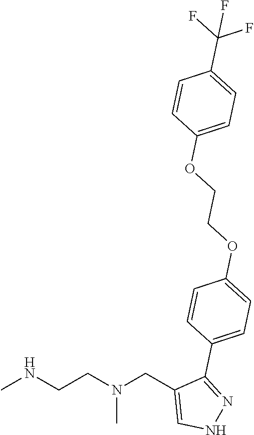 [SMILES: CNCCN(C)Cc1c[nH]nc1-c1ccc(OCCOc2ccc(C(F)(F)F)cc2)cc1] | 449.1 | 0.00608 | 0.00977 | 0.0366 | 0.32776 |
| 139 | 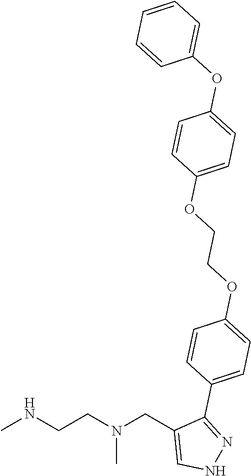 [SMILES: CNCCN(C)Cc1c[nH]nc1-c1ccc(OCCOc2ccc(Oc3ccccc3)cc2)cc1] | 473.3 | 0.00479 | 0.00343 | 0.01924 | 0.32824 |
| 140 | 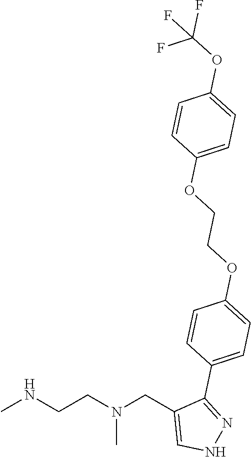 [SMILES: CNCCN(C)Cc1c[nH]nc1-c1ccc(OCCOc2ccc(OC(F)(F)F)cc2)cc1] | 465.25 | 0.00683 | 0.00703 | 0.05262 | 0.47 |
| 141 | 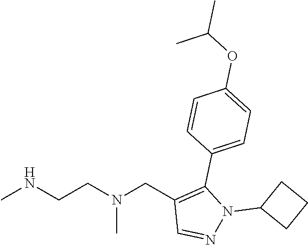 [SMILES: CNCCN(C)Cc1cnn(C2CCC2)c1-c1ccc(OC(C)C)cc1] | — | 0.06581 | 0.08691 | 0.05598 | 0.496 |
| 142 | 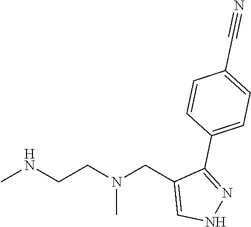 [SMILES: CNCCN(C)Cc1c[nH]nc1-c1ccc(C#N)cc1] | 270.05 | 0.01614 | 0.02705 | 0.02312 | 0.541 |
| 143 | 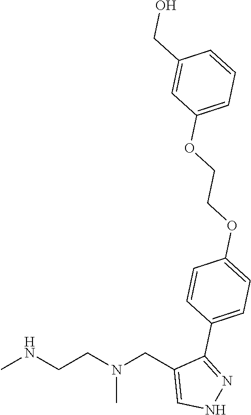 [SMILES: CNCCN(C)Cc1c[nH]nc1-c1ccc(OCCOc2cccc(CO)c2)cc1] | 411.35 | 0.00669 | 0.00448 | 0.02577 | 0.54428 |
| 144 | 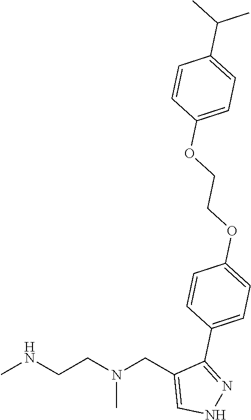 [SMILES: CNCCN(C)Cc1c[nH]nc1-c1ccc(OCCOc2ccc(C(C)C)cc2)cc1] | 423.15 | 0.00643 | 0.00574 | 0.03003 | 0.65458 |
| 145 | 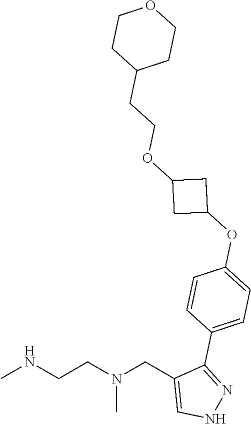 [SMILES: CNCCN(C)Cc1c[nH]nc1-c1ccc(OC2CC(OCCC3CCOCC3)C2)cc1] | 443.3 | 0.11883 | 0.01007 | 0.22297 | 1.039 |
| 146 | 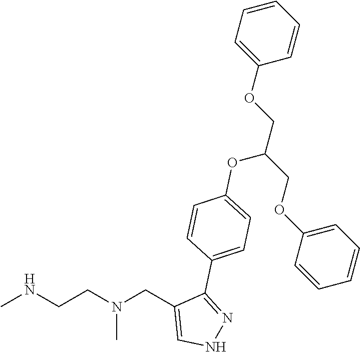 [SMILES: CNCCN(C)Cc1c[nH]nc1-c1ccc(OC(COc2ccccc2)COc2ccccc2)cc1] | 487.35 | 0.00732 | 0.00624 | 0.03747 | 1.40277 |
| 147 | 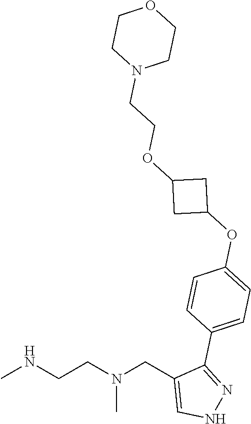 [SMILES: CNCCN(C)Cc1c[nH]nc1-c1ccc(OC2CC(OCCN3CCOCC3)C2)cc1] | 444.1 | 0.11057 | 0.0164 | 0.1621 | 4.237 |
| 148 | 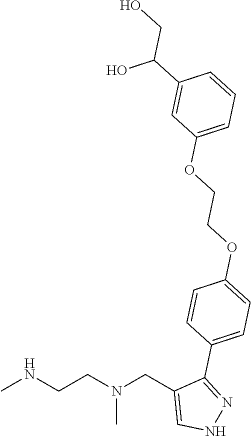 [SMILES: CNCCN(C)Cc1c[nH]nc1-c1ccc(OCCOc2cccc(C(O)CO)c2)cc1] | 441.1 | 0.00822 | 0.00632 | 0.03982 | 8.68816 |
| 149 | 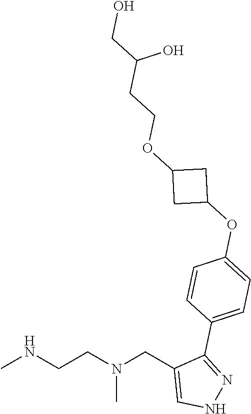 [SMILES: CNCCN(C)Cc1c[nH]nc1-c1ccc(OC2CC(OCCC(O)CO)C2)cc1] | 419.3 | 0.17246 | 0.02861 | 0.34193 | >20 |
| 150 | 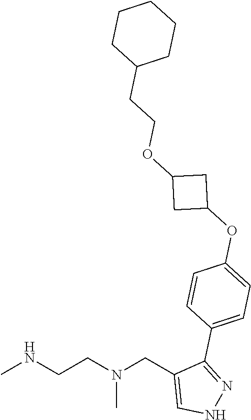 [SMILES: CNCCN(C)Cc1c[nH]nc1-c1ccc(OC2CC(OCCC3CCCCC3)C2)cc1] | 441.3 | 0.02996 | 0.00465 | 0.05986 | >20 |
| 151 | 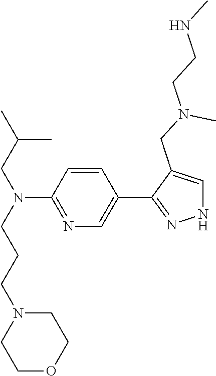 [SMILES: CNCCN(C)Cc1c[nH]nc1-c1ccc(N(CCCN2CCOCC2)CC(C)C)nc1] | 444.3 | 0.01494 | 2.5416 | 0.03119 | 1.33651 |
| 152 | 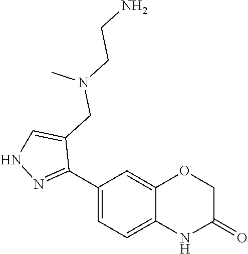 [SMILES: CN(CCN)Cc1c[nH]nc1-c1ccc2c(c1)OCC(=O)N2] | — | 0.06544 | 0.00828 | 0.45917 | >20 |
| 153 | 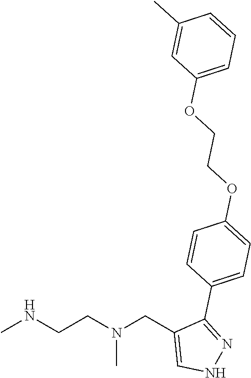 [SMILES: CNCCN(C)Cc1c[nH]nc1-c1ccc(OCCOc2cccc(C)c2)cc1] | — | 0.0043 | 0.00315 | 0.01334 | 0.17756 |
| 154 | 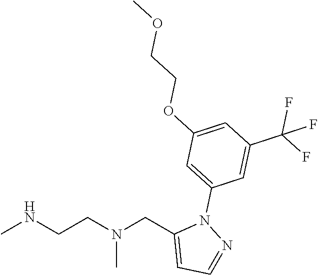 [SMILES: CNCCN(C)Cc1ccnn1-c1cc(OCCOC)cc(C(F)(F)F)c1] | — | 2.03126 | >10.0 uM | 8.45043 | >20 uM |
| 155 | 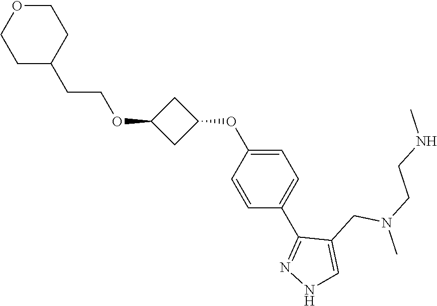 [SMILES: CNCCN(C)Cc1c[nH]nc1-c1ccc(O[C@H]2C[C@H](OCCC3CCOCC3)C2)cc1] | 443.3 | 0.11883 | 0.01007 | 0.22297 | 1.039 |

In certain embodiments, a provided compound inhibits an RMT (e.g., PRMT1, PRMT3, CARM1, PRMT6, and/or PRMT8). In certain embodiments, a provided compound inhibits wild-type PRMT1, PRMT3, CARM1, PRMT6, and/or PRMT8. In certain embodiments, a provided compound inhibits a mutant RMT. In certain embodiments, a provided compound inhibits PRMT1, PRMT3, CARM1, PRMT6, and/or PRMT8, e.g., as measured in an assay described herein. In certain embodiments, the RMT is from a human. In certain embodiments, a provided compound inhibits an RMT (e.g., PRMT1, PRMT3, CARM1, PRMT6, and/or PRMT8) at an IC₅₀ less than or equal to 10 μM. In certain embodiments, a provided compound inhibits an RMT (e.g., PRMT1, PRMT3, CARM1, PRMT6, and/or PRMT8) at an IC₅₀ less than or equal to 1 μM. In certain embodiments, a provided compound inhibits an RMT (e.g., PRMT1, PRMT3, CARM1, PRMT6, and/or PRMT8) at an IC₅₀ less than or equal to 0.1 μM. In certain embodiments, a provided compound inhibits an RMT (e.g., PRMT1, PRMT3, CARM1, PRMT6, and/or PRMT8) at an IC50 less than or equal to 0.01 μM. In certain embodiments, a provided compound inhibits an RMT (e.g., PRMT1, PRMT3, CARM1, PRMT6, and/or PRMT8) in a cell at an EC₃₀ less than or equal to 10 μM. In certain embodiments, a provided compound inhibits an RMT (e.g., PRMT1, PRMT3, CARM1, PRMT6, and/or PRMT8) in a cell at an EC₃₀ less than or equal to 12 μM. In certain embodiments, a provided compound inhibits an RMT (e.g., PRMT1, PRMT3, CARM1, PRMT6, and/or PRMT8) in a cell at an EC₃₀ less than or equal to 3 μM. In certain embodiments, a provided compound inhibits PRMT1 in a cell at an EC₃₀ less than or equal to 12 μM. In certain embodiments, a provided compound inhibits PRMT1 in a cell at an EC₃₀ less than or equal to 3 μM. In certain embodiments, a provided compound inhibits an RMT (e.g., PRMT1, PRMT3, CARM1, PRMT6, and/or PRMT8) in a cell at an EC₃₀ less than or equal to 1 μM. In certain embodiments, a provided compound inhibits an RMT (e.g., PRMT1, PRMT3, CARM1, PRMT6, and/or PRMT8) in a cell at an EC₃₀ less than or equal to 0.1 μM. In certain embodiments, a provided compound inhibits cell proliferation at an EC₅₀ less than or equal to 10 μM. In certain embodiments, a provided compound inhibits cell proliferation at an EC₅₀ less than or equal to 1 μM. In certain embodiments, a provided compound inhibits cell proliferation at an EC₅₀ less than or equal to 0.1 μM.

It will be understood by one of ordinary skill in the art that the RMT can be wild-type, or any mutant or variant.

The present disclosure provides pharmaceutical compositions comprising a compound described herein, e.g., a compound of Table 1, or a pharmaceutically acceptable salt thereof, as described herein, and optionally a pharmaceutically acceptable excipient. It will be understood by one of ordinary skill in the art that the compounds described herein, or salts thereof, may be present in various forms, such as amorphous, hydrates, solvates, or polymorphs. In certain embodiments, a provided composition comprises two or more compounds described herein. In certain embodiments, a compound described herein, or a pharmaceutically acceptable salt thereof, is provided in an effective amount in the pharmaceutical composition. In certain embodiments, the effective amount is a therapeutically effective amount. In certain embodiments, the effective amount is an amount effective for inhibiting an RMT (e.g., PRMT1, PRMT3, CARM1, PRMT6, and/or PRMT8). In certain embodiments, the effective amount is an amount effective for treating an RMT-mediated disorder (e.g., a PRMT1-, PRMT3-, CARM1-, PRMT6-, and/or PRMT8-mediated disorder). In certain embodiments, the effective amount is a prophylactically effective amount. In certain embodiments, the effective amount is an amount effective to prevent an RMT-mediated disorder.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing a compound described herein (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

In some embodiments, a pharmaceutical composition described herein is sterilized.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxylpropyl cellulose, hydroxylpropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxylpropyl cellulose, hydroxylpropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds described herein are mixed with solubilizing agents such as Cremophor™, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a provided compound may include ointments, pastes, creams, lotions, gels, powders, solutions, sprays, inhalants and/or patches. Generally, the active ingredient is admixed under sterile conditions with a pharmaceutically acceptable carrier and/or any desired preservatives and/or buffers as can be required. Additionally, the present disclosure encompasses the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A provided pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A provided pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A provided pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In certain embodiments, the additional therapeutically active agent is a compound of Table 1. In certain embodiments, the additional therapeutically active agent is not a compound of Table 1. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, an additional therapeutically active agent is prednisolone, dexamethasone, doxorubicin, vincristine, mafosfamide, cisplatin, carboplatin, Ara-C, rituximab, azacitadine, panobinostat, vorinostat, everolimus, rapamycin, ATRA (all-trans retinoic acid), daunorubicin, decitabine, Vidaza, mitoxantrone, or IBET-151.

Also encompassed by the present disclosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

Compounds and compositions described herein are generally useful for the inhibition of RMT (e.g., PRMT1, PRMT3, CARM1, PRMT6, and/or PRMT8). In some embodiments, methods of treating an RMT-mediated disorder in a subject are provided which comprise administering an effective amount of a compound of Table 1, or a pharmaceutically acceptable salt thereof), to a subject in need of treatment. In certain embodiments, the effective amount is a therapeutically effective amount. In certain embodiments, the effective amount is a prophylactically effective amount. In certain embodiments, the subject is suffering from a RMT-mediated disorder. In certain embodiments, the subject is susceptible to a RMT-mediated disorder.

As used herein, the term “RMT-mediated disorder” means any disease, disorder, or other pathological condition in which an RMT (e.g., PRMT1, PRMT3, CARM1, PRMT6, and/or PRMT8) is known to play a role. Accordingly, in some embodiments, the present disclosure relates to treating or lessening the severity of one or more diseases in which an RMT is known to play a role.

In some embodiments, the present disclosure provides a method of inhibiting an RMT comprising contacting the RMT with an effective amount of a compound of Table 1, or a pharmaceutically acceptable salt thereof. The RMT may be purified or crude, and may be present in a cell, tissue, or subject. Thus, such methods encompass both inhibition of in vitro and in vivo RMT activity. In certain embodiments, the method is an in vitro method, e.g., such as an assay method. It will be understood by one of ordinary skill in the art that inhibition of an RMT does not necessarily require that all of the RMT be occupied by an inhibitor at once. Exemplary levels of inhibition of an RMT (e.g., PRMT1, PRMT3, CARM1, PRMT6, and/or PRMT8) include at least 10% inhibition, about 10% to about 25% inhibition, about 25% to about 50% inhibition, about 50% to about 75% inhibition, at least 50% inhibition, at least 75% inhibition, about 80% inhibition, about 90% inhibition, and greater than 90% inhibition.

In some embodiments, provided is a method of inhibiting RMT activity in a subject in need thereof (e.g., a subject diagnosed as having an RMT-mediated disorder) comprising administering to the subject an effective amount of a compound of Table 1, or a pharmaceutically acceptable salt thereof, or a pharmaceutical composition thereof.

In certain embodiments, provided is a method of modulating gene expression in a cell which comprises contacting a cell with an effective amount of a compound of Table 1, or a pharmaceutically acceptable salt thereof. In certain embodiments, the cell is in culture in vitro. In certain embodiments, the cell is in an animal, e.g., a human. In certain embodiments, the cell is in a subject in need of treatment.

In certain embodiments, provided is a method of modulating transcription in a cell which comprises contacting a cell with an effective amount of a compound of Table 1, or a pharmaceutically acceptable salt thereof. In certain embodiments, the cell is in culture in vitro. In certain embodiments, the cell is in an animal, e.g., a human. In certain embodiments, the cell is in a subject in need of treatment.

In certain embodiments, a method is provided of selecting a therapy for a subject having a disease associated with an RMT-mediated disorder or mutation comprising the steps of determining the presence of an RMT-mediated disorder or gene mutation in an RMT gene (e.g., a PRMT1, PRMT3, CARM1, PRMT6, and/or PRMT8 gene) or and selecting, based on the presence of an RMT-mediated disorder a gene mutation in the RMT gene a therapy that includes the administration of a provided compound. In certain embodiments, the disease is cancer.

In certain embodiments, a method of treatment is provided for a subject in need thereof comprising the steps of determining the presence of an RMT-mediated disorder or a gene mutation in the RMT gene and treating the subject in need thereof, based on the presence of a RMT-mediated disorder or gene mutation in the RMT gene with a therapy that includes the administration of a provided compound. In certain embodiments, the subject is a cancer patient.

In some embodiments, a compound provided herein is useful in treating a proliferative disorder, such as cancer. For example, while not being bound to any particular mechanism, protein arginine methylation by PRMTs is a modification that has been implicated in signal transduction, gene transcription, DNA repair and mRNA splicing, among others; and overexpression of PRMTs within these pathways is often associated with various cancers. Thus, compounds which inhibit the action of PRMTs, as provided herein, are effective in the treatment of cancer.

In some embodiments, compounds provided herein are effective in treating cancer through the inhibition of PRMT1. For example, PRMT1 overexpression has been observed in various human cancers, including, but not limited to, breast cancer, prostate cancer, lung cancer, colon cancer, bladder cancer, and leukemia. In one example, PRMT1 specifically deposits an asymmetric dimethylarginine (aDMA) mark on histone H4 at arginine 3 (H4R3me2a), and this mark is associated with transcription activation. In prostate cancer, the methylation status of H4R3 positively correlates with increasing tumor grade and can be used to predict the risk of prostate cancer recurrence (Seligson et al., Nature 2005 435, 1262-1266). Thus, in some embodiments, inhibitors of PRMT1, as described herein, are useful in treating cancers associated with the methylation status of H4R3, e.g., prostate cancer. Additionally, the methylarginine effector molecule TDRD3 interacts with the H4R3me2a mark, and overexpression of TDRD3 is linked to poor prognosis for the survival of patients with breast cancer (Nagahata et al., Cancer Sci. 2004 95, 218-225). Thus, in some embodiments, inhibitors of PRMT1, as described herein, are useful in treating cancers associated with overexpression of TDRD3, e.g., breast cancer, as inhibition of PRMT1 leads to a decrease in methylation of H4R3, thereby preventing the association of overexpressed TDRD3 with H4R3me2a. In other examples, PRMT1 is known to have non-histone substrates. For example, PRMT1, when localized to the cytoplasm, methylates proteins that are involved in signal transduction pathways, e.g., the estrogen receptor (ER). The expression status of ER in breast cancer is critical for prognosis of the disease, and both genomic and non-genomic ER pathways have been implicated in the pathogenesis of breast cancer. For example, it has been shown that PRMT1 methylates ERα, and that ERα methylation is required for the assembly of ERα with SRC (a proto-oncogene tyrosine-protein kinase) and focal adhesion kinase (FAK). Further, the silencing of endogenous PRMT1 resulted in the inability of estrogen to activate AKT. These results suggested that PRMT1-mediated ERα methylation is required for the activation of the SRC-PI3K-FAK cascade and AKT, coordinating cell proliferation and survival. Thus, hypermethylation of ERα in breast cancer is thought to cause hyperactivation of this signaling pathway, providing a selective survival advantage to tumor cells (Le Romancer et al., Mol. Cell 2008 31, 212-221; Le Romancer et al., Steroids 2010 75, 560-564). Accordingly, in some embodiments, inhibitors of PRMT1, as described herein, are useful in treating cancers associated with ERα methylation, e.g., breast cancer. In yet another example, PRMT1 has been shown to be involved in the regulation of leukemia development. For example, SRC-associated in mitosis 68 kDa protein (SAM68; also known as KHDRBS1) is a well-characterized PRMT1 substrate, and when either SAM68 or PRMT1 is fused directly to the myeloid/lymphoid leukemia (MLL) gene, these fusion proteins can activate MLL oncogenic properties, implying that the methylation of SAM68 by PRMT1 is a critical signal for the development of leukemia (Cheung et al., Nature Cell Biol. 2007 9, 1208-1215). Accordingly, in some embodiments, inhibitors of PRMT1, as described herein, are useful in treating cancers associated with SAM68 methylation, e.g., leukemia. In still another example, PRMT1 is implicated in leukemia development through its interaction with AE9a, a splice isoform of AML1-ETO (Shia et al., Blood 2012 119:4953-62). Knockdown of PRMT1 affects expression of certain AE9a-activated genes and suppresses AE9a's self-renewal capability. It has also been shown that AE9a recruits PRMT1 to AE9a activated gene promoters, which leads to increased H4 Arg3 methylation, H3 Lys9/14 acetylation, and transcription activated. Accordingly, in some embodiments, inhibitors of PRMT1, as described herein, are useful in treating cancers associated with AML1-ETO, e.g., leukemia. Thus, without being bound by any particular mechanism, the inhibition of PRMT1, e.g., by compounds described herein, is beneficial in the treatment of cancer.

In some embodiments, compounds provided herein are effective in treating cancer through the inhibition of PRMT3. In one example, the DAL1 tumor suppressor protein has been shown to interact with PRMT3 and inhibits its methyltransferase activity (Singh et al., Oncogene 2004 23, 7761-7771). Epigenetic downregulation of DAL1 has been reported in several cancers (e.g., meningiomas and breast cancer), thus PRMT3 is expected to display increased activity, and cancers that display DAL1 silencing may, in some aspects, be good targets for PRMT3 inhibitors, e.g., those described herein. Thus, without being bound by any particular mechanism, the inhibition of PRMT3, e.g., by compounds described herein, is beneficial in the treatment of cancer.

In some embodiments, compounds provided herein are effective in treating cancer through the inhibition of PRMT4, also known as CARM1. For example, PRMT4 levels have been shown to be elevated in castration-resistant prostate cancer (CRPC), as well as in aggressive breast tumors (Hong et al., Cancer 2004 101, 83-89; Majumder et al., Prostate 2006 66, 1292-1301). Thus, in some embodiments, inhibitors of PRMT4, as described herein, are useful in treating cancers associated with PRMT4 overexpression. PRMT4 has also been shown to affect ERα-dependent breast cancer cell differentiation and proliferation (Al-Dhaheri et al., Cancer Res. 2011 71, 2118-2128), thus in some aspects PRMT4 inhibitors, as described herein, are useful in treating ERα-dependent breast cancer by inhibiting cell differentiation and proliferation. In another example, PRMT4 has been shown to be recruited to the promoter of E2F1 (which encodes a cell cycle regulator) as a transcriptional co-activator (Frietze et al., Cancer Res. 2008 68, 301-306). Thus, PRMT4-mediated upregulation of E2F1 expression may contribute to cancer progression and chemoresistance as increased abundance of E2F1 triggers invasion and metastasis by activating growth receptor signaling pathways, which in turn promote an antiapoptotic tumor environment (Engelmann and Pützer, Cancer Res 2012 72; 571). Accordingly, in some embodiments, the inhibition of PRMT4, e.g., by compounds provided herein, is useful in treating cancers associated with E2F1 upregulation. Thus, without being bound by any particular mechanism, the inhibition of PRMT4, e.g., by compounds described herein, is beneficial in the treatment of cancer.

In some embodiments, compounds provided herein are effective in treating cancer through the inhibition of PRMT6. For example, PRMT6 has been reported to be overexpressed in a number of cancers, e.g., bladder and lung cancer (Yoshimatsu et al., Int. J. Cancer 2011 128, 562-573). Thus, in some embodiments, the inhibition of PRMT6, by compounds provided herein, is useful in treating cancers associated with PRMT6 overexpression. In some aspects, PRMT6 is primarily thought to function as a transcriptional repressor, although it has also been reported that PRMT6 functions as a co-activator of nuclear receptors. For example, as a transcriptional repressor, PRMT6 suppresses the expression of thrombospondin 1 (TSP1; also known as THBS1; a potent natural inhibitor of angiogenesis and endothelial cell migration) and p21 (a natural inhibitor of cyclin dependent kinase), thereby contributing to cancer development and progression (Michaud-Levesque and Richard, J. Biol. Chem. 2009 284, 21338-21346; Kleinschmidt et al., PLoS ONE 2012 7, e41446). Accordingly, in some embodiments, the inhibition of PRMT6, by compounds provided herein, is useful in treating cancer by preventing the repression of THBs1 and/or p21. Thus, without being bound by any particular mechanism, the inhibition of PRMT6, e.g., by compounds described herein, is beneficial in the treatment of cancer.

In some embodiments, compounds provided herein are effective in treating cancer through the inhibition of PRMT8. For example, deep-sequencing efforts of cancer genomes (e.g., COSMIC) have revealed that of all the PRMTs, PRMT8 is reported to be the most mutated. Of 106 sequenced genomes, 15 carry mutations in the PRMT8 coding region, and nine of these result in an amino acid change (Forbes et al., Nucleic Acids Res. 2011 39, D945-D950). Because of its high rate of mutation in cancer, PRMT8 is thought to contribute to the initiation or progression of cancer. Thus, without being bound by any particular mechanism, the inhibition of PRMT8, e.g., by compounds described herein, is beneficial in the treatment of cancer.

In some embodiments, compounds described herein are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (e.g., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, a compound provided herein is useful in treating diseases associated with increased levels of circulating asymmetric dimethylarginine (aDMA), e.g., cardiovascular disease, diabetes, kidney failure, renal disease, pulmonary disease, etc. Circulating aDMA is produced by the proteolysis of asymmetrically dimethylated proteins. PRMTs which mediate aDMA methylation include, e.g., PRMT1, PRMT3, PRMT4, PRMT6, and PRMT8. aDMA levels are directly involved in various diseases as aDMA is an endogenous competitive inhibitor of nitric oxide synthase (NOS), thereby reducing the production of nitric oxide (NO) (Vallance et al., J. Cardiovasc. Pharmacol. 1992 20(Suppl. 12):560-2). NO functions as a potent vasodilator in endothelial vessels, and as such inhibiting its production has major consequences on the cardiovascular system. For example, since PRMT1 is a major enzyme that generates aDMA, the dysregulation of its activity is likely to regulate cardiovascular diseases (Boger et al., Ann. Med. 2006 38:126-36), and other pathophysiological conditions such as diabetes mellitus (Sydow et al., Vasc. Med. 2005 10(Suppl. 1):535-43), kidney failure (Vallance et al., Lancet 1992 339:572-5), and chronic pulmonary diseases (Zakrzewicz et al., BMC Putin. Med. 2009 9:5). Additionally, it has been demonstrated that the expression of PRMT1 and PRMT3 are increased in coronary heart disease (Chen et al., Basic Res. Cardiol. 2006 101:346-53). In another example, aDMA elevation is seen in patients with renal failure, due to impaired clearance of this metabolite from the circulation (Jacobi et al., Am. J. Nephrol. 2008 28:224-37). Thus, circulating aDMA levels is observed in many pathophysiological situations. Accordingly, without being bound by any particular mechanism, the inhibition of PRMTs, e.g., by compounds described herein, results in the decrease of circulating aDMA, which is beneficial in the treatment of diseases associated with increased levels of circulating aDMA, e.g., cardiovascular disease, diabetes, kidney failure, renal disease, pulmonary disease, etc. In certain embodiments, a compound described herein is useful for treating or preventing vascular diseases.

In some embodiments, a compound provided herein is useful in treating metabolic disorders. For example, PRMT1 has been shown to enhance mRNA levels of FoxO1 target genes in gluconeogenesis, which results in increased hepatic glucose production, and knockdown of PRMT promotes inhibition of FoxO1 activity and thus inhibition of hepatic gluconeogenesis (Choi et al., Hepatology 2012 56:1546-56). Additionally, genetic haploinsufficiency of Prmt1 has been shown to reduce blood glucose levels in mouse models. Thus, without being bound by any particular mechanism, the inhibition of PRMT1, e.g., by compounds described herein, is beneficial in the treating of metabolic disorders, such as diabetes. In some embodiments, a provided compound is useful in treating type I diabetes. In some embodiments, a provided compound is useful in treating type II diabetes.

In some embodiments, a compound provided herein is useful in treating muscular dystrophies. For example, PRMT1, as well as PRMT3 and PRMT6, methylate the nuclear poly(A)-binding protein (PABPN1) in a region located near its C-terminus (Perreault et al., J. Biol. Chem. 2007 282:7552-62). This domain is involved in the aggregation of the PABPN1 protein, and abnormal aggregation of this protein is involved in the disease oculopharyngeal muscular dystrophy (Davies et al., Int. J. Biochem. Cell. Biol. 2006 38:1457-62). Thus, without being bound by any particular mechanism, the inhibition of PRMTs, e.g., by compounds described herein, is beneficial in the treatment of muscular dystrophies, e.g., oculopharyngeal muscular dystrophy, by decreasing the amount of methylation of PABPN1, thereby decreasing the amount of PABPN1 aggregation.

CARM1 is also the most abundant PRMT expressed in skeletal muscle cells, and has been found to selectively control the pathways modulating glycogen metabolism, and associated AMPK (AMP-activated protein kinase) and p38 MAPK (mitogen-activated protein kinase) expression. See, e.g., Wang et al., Biochem (2012) 444:323-331. Thus, in some embodiments, inhibitors of CARM1, as described herein, are useful in treating metabolic disorders, e.g., for example skeletal muscle metabolic disorders, e.g., glycogen and glucose metabolic disorders. Exemplary skeletal muscle metabolic disorders include, but are not limited to, Acid Maltase Deficiency (Glycogenosis type 2; Pompe disease), Debrancher deficiency (Glycogenosis type 3), Phosphorylase deficiency (McArdle's; GSD 5), X-linked syndrome (GSD9D), Autosomal recessive syndrome (GSD9B), Tarui's disease (Glycogen storage disease VII; GSD 7), Phosphoglycerate Mutase deficiency (Glycogen storage disease X; GSDX; GSD 10), Lactate dehydrogenase A deficiency (GSD 11), Branching enzyme deficiency (GSD 4), Aldolase A (muscle) deficiency, β-Enolase deficiency, Triosephosphate isomerase (TIM) deficiency, Lafora's disease (Progressive myoclonic epilepsy 2), Glycogen storage disease (Muscle, Type 0, Phosphoglucomutase 1 Deficiency (GSD 14)), and Glycogenin Deficiency (GSD 15).

In some embodiments, a compound provided herein is useful in treating autoimmune disease. For example, several lines of evidence strongly suggest that PRMT inhibitors may be valuable for the treatment of autoimmune diseases, e.g., rheumatoid arthritis. PRMTs are known to modify and regulate several critical immunomodulatory proteins. For example, post-translational modifications (e.g., arginine methylation), within T cell receptor signaling cascades allow T lymphocytes to initiate a rapid and appropriate immune response to pathogens. Co-engagement of the CD28 costimulatory receptor with the T cell receptor elevates PRMT activity and cellular protein arginine methylation, including methylation of the guanine nucleotide exchange factor Vavl (Blanchet et al., J. Exp. Med. 2005 202:371-377). PRMT inhibitors are thus expected to diminish methylation of the guanine exchange factor Vavl, resulting in diminished IL-2 production. In agreement, siRNA directed against PRMT5 was shown to both inhibit NFAT-driven promoter activity and IL-2 secretion (Richard et al., Biochem J. 2005 388:379-386). In another example, PRMT1 is known to cooperate with PRMT4 to enhance NFkB p65-driven transcription and facilitate the transcription of p65 target genes like TNFα (Covic et al., Embo. J. 2005 24:85-96). Thus, in some embodiments, PRMT1 and/or PRMT4 inhibitors, e.g., those described herein, are useful in treating autoimmune disease by decreasing the transcription of p65 target genes like TNFα. These examples demonstrate an important role for arginine methylation in inflammation. Thus, without being bound by any particular mechanism, the inhibition of PRMTs, e.g., by compounds described herein, is beneficial in the treatment of autoimmune diseases.

In some embodiments, a compound provided herein is useful in treating neurological disorders, such as amyotrophic lateral sclerosis (ALS). For example, a gene involved in ALS, TLS/FUS, often contains mutated arginines in certain familial forms of this disease (Kwiatkowski et al., Science 2009 323:1205-8). These mutants are retained in the cytoplasm, which is similar to reports documenting the role arginine methylation plays in nuclear-cytoplasmic shuffling (Shen et al., Genes Dev. 1998 12:679-91). This implicates PRMT, e.g., PRMT1, function in this disease, as it was demonstrated that TLS/FUS is methylated on at least 20 arginine residues (Rappsilber et al., Anal. Chem. 2003 75:3107-14). Thus, in some embodiments, the inhibition of PRMTs, e.g., by compounds provided herein, are useful in treating ALS by decreasing the amount of TLS/FUS arginine methylation.

### EXAMPLES

In order that the invention described herein may be more fully understood, the following examples are set forth. It should be understood that these examples are for illustrative purposes only and are not to be construed as limiting this invention in any manner.

### Synthetic Methods

Compounds described herein may be prepared following the experimental procedures and general methods as described in PCT/US2014/029710 and PCT/US2014/029583, each of which is incorporated herein by reference.

### Example 1

### Synthesis of N1,N2-dimethyl-N1-((3-(4-(3-(2-(tetrahydro-2H-pyran-4-yl)ethoxy)cyclobutoxy)phenyl)-1H-pyrazol-4-yl)methyl)ethane-1,2-diamine bis(2,2,2-trifluoroacetate) (Compound 155)

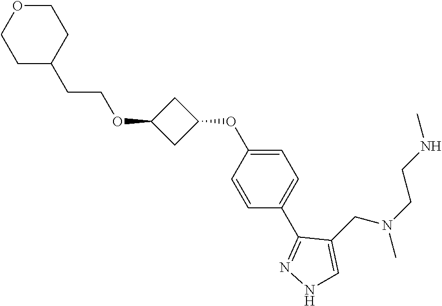 [SMILES: CNCCN(C)Cc1c[nH]nc1-c1ccc(O[C@H]2C[C@H](OCCC3CCOCC3)C2)cc1]

### Step 1: 3-(benzyloxy)cyclobutanol

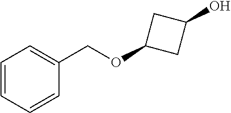 [SMILES: O[C@H]1C[C@@H](OCc2ccccc2)C1]

Into a 100-mL round-bottom flask, was placed 3-(benzyloxy)cyclobutan-1-one (7 g, 39.72 mmol, 1.00 equiv), methanol (50 mL). Then the mixture was cooled to 0 degree C. and NaBH₄ (2.3 g, 62.46 mmol, 1.57 equiv) was added in batches over 10 mins. The resulting solution was stirred overnight at room temperature. The reaction was then quenched by the addition of 50 mL of NH₄Cl (sat. aq.). The resulting mixture was concentrated under vacuum. The resulting solution was extracted with ethyl acetate (50 mL×5). The organic phase was washed with 3×50 mL of brine (sat.), and then it was collected and dried over anhydrous sodium sulfate and concentrated under vacuum. This resulted in 6.9 g (97%) of 3-(benzyloxy)cyclobutan-1-ol as light yellow oil.

### Step 2: 3-(benzyloxy)cyclobutyl 4-methylbenzenesulfonate

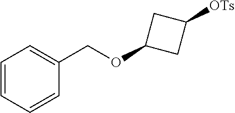 [SMILES: Cc1ccc(S(=O)(=O)O[C@H]2C[C@@H](OCc3ccccc3)C2)cc1]

Into a 100-mL round-bottom flask, was placed 3-(benzyloxy)cyclobutan-1-ol (6.5 g, 36.47 mmol, 1.00 equiv), dichloromethane (50 mL), triethylamine (25 mL). Cooled to 0 degree C., this was followed by the addition of a solution of 4-methylbenzene-1-sulfonyl chloride (13.8 g, 72.39 mmol, 1.98 equiv) in dichloromethane (10 mL) by dropwise with stirring over 30 mins. The resulting solution was stirred overnight at room temperature. The resulting solution was diluted with 30 mL of CH₂Cl₂. The resulting mixture was washed with 3×30 mL of brine (sat.). The mixture was dried over anhydrous sodium sulfate and concentrated under vacuum. The residue was applied onto a silica gel column with ethyl acetate/petroleum ether (0%-10%). The collected fractions were combined and concentrated under vacuum. This resulted in 10.5 g (87%) of 3-(benzyloxy)cyclobutyl 4-methylbenzene-1-sulfonate as a yellow solid.

### Step 3: Tert-butyl 2-(((3-(4-(3-(benzyloxy)cyclobutoxy)phenyl)-1-(tetrahydro-2H-pyran-2-yl)-1H-pyrazol-4-yl)methyl)(methyl)amino)ethyl(methyl)carbamate

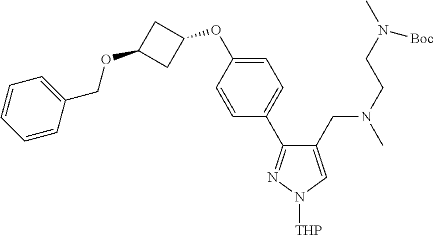

Into a 100-mL round-bottom flask, was placed tert-butyl N-[2-([3-(4-hydroxyphenyl)-1-(oxan-2-yl)-1H-pyrazol-4-yl]methyl(methyl)amino)ethyl]-N-methylcarbamate (9 g, 20.24 mmol, 1.00 equiv), 3-(benzyloxy)cyclobutyl 4-methylbenzene-1-sulfonate (8.1 g, 24.37 mmol, 1.20 equiv), Cs₂CO₃ (20 g, 61.19 mmol, 3.02 equiv) and N,N-dimethylformamide (100 mL). The resulting solution was stirred for 3 h at 100° C. in an oil bath. The reaction was then quenched by the addition of 50 mL of water. The resulting solution was extracted with ethyl acetate (50 mL×3). The resulting mixture was washed with 3×50 mL of brine (sat.). The mixture was dried over anhydrous sodium sulfate and concentrated under vacuum. The residue was applied onto a silica gel column with ethyl acetate/petroleum ether (0%-20%). The collected fractions were combined and concentrated under vacuum. This resulted in 10.5 g (86%) of tert-butyl N-(2-[[(3-[4-[3-(benzyloxy)cyclobutoxy]phenyl]-1-(oxan-2-yl)-1H-pyrazol-4-yl)methyl](methyl)amino]ethyl)-N-methylcarbamate as yellow oil. LCMS (Method A, ESI): RT=1.43 min, m/z=605.4 [M+H]⁺.

### Step 4: Tert-butyl 2-(((3-(4-(3-hydroxycyclobutoxy)phenyl)-1-(tetrahydro-2H-pyran-2-yl)-1H-pyrazol-4-yl)methyl)(methyl)amino)ethyl(methyl)carbamate

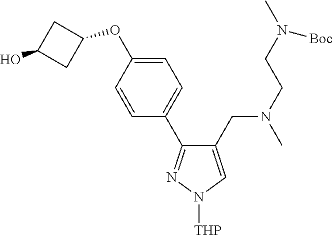

Into a 1-L round-bottom flask, was placed tert-butyl N-(2-[[(3-[4-[3-(benzyloxy)cyclobutoxy]phenyl]-1-(oxan-2-yl)-1H-pyrazol-4-yl)methyl](methyl)amino]ethyl)-N-methylcarbamate (3 g, 4.96 mmol, 1.00 equiv), THF (500 mL), 10% Palladium carbon (3 g) and hydrochloric acid (12N, 0.7 mL). Then hydrogen (gas) was introduced into mixture and maintained at 2 atm. The resulting solution was stirred for 4 h at room temperature. The solids were filtered out. The pH value of the solution was adjusted to 8 with K₂CO₃ (sat. aq.). The resulting solution concentrated under vacuum. The residue was applied onto a silica gel column with ethyl acetate/petroleum ether (0%-75%). The collected fractions were combined and concentrated under vacuum. This resulted in 2.13 g (83%) of tert-butyl N-[2-[([3-[4-(3-hydroxycyclobutoxy)phenyl]-1-(oxan-2-yl)-1H-pyrazol-4-yl]methyl)(methyl)amino]ethyl]-N-methylcarbamate as light yellow oil. LCMS (Method B, ESI): RT=0.99 min, m/z=515.4 [M+H]⁺

Step 5: Tert-butyl methyl(2-(methyl((1-(tetrahydro-2H-pyran-2-yl)-3-(4-(3-(2-(tetrahydro-2H-pyran-4-yl)ethoxy)cyclobutoxy)phenyl)-1H-pyrazol-4-yl)methyl)amino)ethyl)carbamate

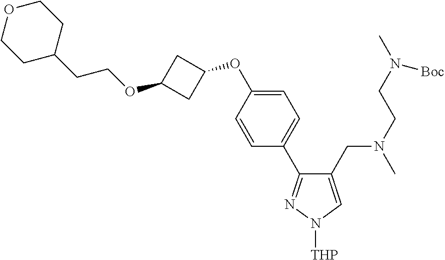

Into a 50-mL 3-necked round-bottom flask, was placed tert-butyl N-[2-[([3-[4-(3-hydroxycyclobutoxy)phenyl]-1-(oxan-2-yl)-1H-pyrazol-4-yl]methyl)(methyl)amino]ethyl]-N-methylcarbamate (500 mg, 0.97 mmol, 1.00 equiv),N,N-dimethylformamide (10 mL). The temperature was cooled to 0° C. To this was added sodium hydride (120 mg, 5.00 mmol, 5.15 equiv, 60% in mineral oil) in batches. The mixture was stirred for 1 h at R.T. Then to the mixture was added 4-(2-bromoethyl)oxane (470 mg, 2.43 mmol, 2.51 equiv). The resulting solution was stirred overnight at room temperature. The reaction was then quenched by the addition of 30 mL of water. The resulting solution was extracted with 3×30 mL of ethyl acetate. The resulting mixture was washed with 3×30 mL of brine. The mixture was dried over anhydrous sodium sulfate and concentrated under vacuum. The residue was applied onto a silica gel column with ethyl acetate/petroleum ether (0%-60%). The collected fractions were combined and concentrated under vacuum. This resulted in 450 mg (74%) of tert-butyl N-methyl-N-[2-[methyl([[1-(oxan-2-yl)-3-(4-[3-[2-(oxan-4-yl)ethoxy]cyclobutoxy]phenyl)-1H-pyrazol-4-yl]methyl])amino]ethyl]carbamate as yellow oil. LCMS (Method A, ESI): RT=1.37 min, m/z=627.4 [M+H].

### Step 6: N1,N2-dimethyl-N1-((3-(4-(3-(2-(tetrahydro-2H-pyran-4-yl)ethoxy)cyclobutoxy)phenyl)-1H-pyrazol-4-yl)methyl)ethane-1,2-diamine bis(2,2,2-trifluoroacetate) (Compound 155)

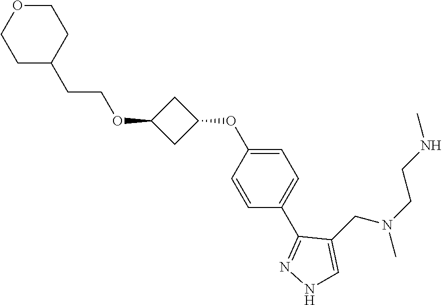 [SMILES: CNCCN(C)Cc1c[nH]nc1-c1ccc(O[C@H]2C[C@H](OCCC3CCOCC3)C2)cc1]

Into a 50-mL round-bottom flask, was placed tert-butyl N-methyl-N-[2-[methyl([[1-(oxan-2-yl)-3-(4-[3-[2-(oxan-4-yl)ethoxy]cyclobutoxy]phenyl)-1H-pyrazol-4-yl]methyl])amino]ethyl]carbamate (450 mg, 0.72 mmol, 1.00 equiv), trifluoroacetic acid (3 mL), dichloromethane (3 mL). The resulting solution was stirred for 2 h at room temperature. The resulting mixture was concentrated under vacuum. The crude product was purified by Prep-HPLC with the following conditions (1#-Pre-HPLC-005(Waters)): Column, Atlantis Prep OBD T3 Column, 19*150 mm, 5 um; mobile phase, water with 0.05% TFA and CH₃CN (up to 3.0% in 10 min, up to 100.0% in 1 min, hold 100.0% in 1 min); Detector, UV 254 nm. This resulted in 198.8 mg (41%) of methyl[2-(methylamino)ethyl][[3-(4-[3-[2-(oxan-4-yl)ethoxy]cyclobutoxy]phenyl)-1H-pyrazol-4-yl]methyl]amine bis(trifluoroacetic acid) as light yellow oil. ¹H-NMR (300 MHz, D20): 6 7.89 (s, 1H), 7.43 (d, J=4.5 Hz, 2H), 6.98 (d, J=4.4 Hz, 2H), 4.94-4.87 (m, 1H), 4.42 (s, 2H), 4.31-4.23 (m, 1H), 3.90-3.85 (m, 2H), 3.47-3.34 (m, 4H), 3.20 (s, 4H), 2.61 (s, 3H), 2.56 (s, 3H), 2.48-2.37 (m, 4H), 1.65-1.57 (m, 3H), 1.50-1.44 (m, 2H), 1.29-1.14 (m, 2H) ppm. LCMS (Method A, ESI): RT=1.23 min, m/z=443.3[M+H]⁺.

### Biological Methods

### PRMT1 Biochemical Assay

General Materials.

S-adenosylmethionine (SAM), S-adenosylhomocysteine (SAH), bicine, Tween20, dimethylsulfoxide (DMSO), bovine skin gelatin (BSG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 36-50 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-RLARRGGVKRISGLI-amide (SEQ ID NO.:1).

Molecular Biology:

Full-length human PRMT1 isoform 1 (NM_001536.5) transcript clone was amplified from an HEK 293 cDNA library, incorporating flanking 5′ sequence encoding a FLAG tag (DYKDDDDK) (SEQ ID NO.:2) fused directly to Met 1 of PRMT1. The amplified gene was subcloned into pFastBacl (Life Technologies) modified to encode an N-terminal GST tag and a TEV cleavage sequence (MSPILGYWKIKGLVQPTRLLLEYLEEKYEEHLYERDEGDKWRNKKFELGLEFPNLP YYIDGDVKLTQSMAIIRYIADKHNMLGGCPKERAEISMLEGAVLDIRYGVSRIAYSK DFETLKVDFLSKLPEMLKMFEDRLCHKTYLNGDHVTHPDFMLYDALDVVLYMDPM CLDAFPKLVCFKKRIEMPQIDKYLKSSKYIAWPLQGWQATFGGGDHPPKSDENLYF QGGNS)(SEQ ID NO.:3) fused to Asp of the Flag tag of PRMT1.

Protein Expression.

Recombinant baculovirus were generated according to Bac-to-Bac kit instructions (Life Technologies). Protein over-expression was accomplished by infecting exponentially growing High Five insect cell culture at 1.5×10⁶ cell/ml with 1:100 ratio of virus. Infections were carried out at 27° C. for 48 hours, harvested by centrifugation, and stored at −80° C. for purification.

Protein Purification.

Expressed full-length human GST-tagged PRMT1 protein was purified from cell paste by glutathione sepharose affinity chromatography after equilibration of the resin with 50 mM phosphate buffer, 200 mM NaCl, 5% glycerol, 5 mM 3-mercaptoethanol, pH7.8 (Buffer A). GST-tagged PRMT1 was eluted with 50 mM Tris, 2 mM glutathione, pH 7.8, dialysed in buffer A and concentrated to 1 mg/mL. The purity of recovered protein was 73%. Reference: Wasilko, D. J. and S. E. Lee: “TIPS: titerless infected-cells preservation and scale-up” Bioprocess J., 5 (2006), pp. 29-32.

Predicted Translations:

GST-tagged PRMT1
(SEQ ID NO.: 4)
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDENLYFQGGNSDYKDDDDKMAAAEAANCIME
NFVATLANGMSLQPPLEEVSCGQAESSEKPNAEDMTSKDYYFDSYAHFGI
HEEMLKDEVRTLTYRNSMFHNRHLFKDKVVLDVGSGTGILCMFAAKAGAR
KVIGIECSSISDYAVKIVKANKLDHVVTIIKGKVEEVELPVEKVDIIISE
WMGYCLFYESMLNTVLYARDKWLAPDGLIFPDRATLYVTAIEDRQYKDYK
IHWWENVYGFDMSCIKDVAIKEPLVDVVDPKQLVTNACLIKEVDIYTVKV
EDLTFTSPFCLQVKRNDYVHALVAYFNIEFTRCHKRTGFSTSPESPYTHW
KQTVFYMEDYLTVKTGEEIFGTIGMRPNAKNNRDLDFTIDLDFKGQLCEL
SCSTDYRMR

General Procedure for PRMT1 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT1, was added to columns 11, 12, 23, 24, rows I-P for the minimum signal control. A cocktail (40 ul) containing the PRMT1 enzyme was added by Multidrop Combi (Thermo-Fisher). The compounds were allowed to incubate with PRMT1 for 30 min at room temperature, then a cocktail (10 ul) containing SAM and peptide was added to initiate the reaction (final volume=51 ul). The final concentrations of the components were as follows: PRMT1 was 0.5 nM, ³H-SAM was 200 nM, non-radiolabeled SAM was 1.5 uM, peptide was 20 nM, SAH in the minimum signal control wells was 1 mM, and the DMSO concentration was 2%. The assays were stopped by the addition of non-radiolabeled SAM (10 ul) to a final concentration of 300 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

### % Inhibition Calculation

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

### Four-Parameter IC50 Fit

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT6 Biochemical Assay

General Materials.

S-adenosylmethionine (SAM), S-adenosylhomocysteine (SAH), bicine, Tween20, dimethylsulfoxide (DMSO), bovine skin gelatin (BSG), sodium butyrate and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 36-50 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-RLARRGGVKRISGLI-amide and contained a monomethylated lysine at position 44 (SEQ ID NO.:5).

Molecular Biology:

Full-length human PRMT6 (NM_018137.2) transcript clone was amplified from an HEK 293 cDNA library, incorporating a flanking 5′ sequence encoding a FLAG tag (MDYKDDDDK) (SEQ ID NO.:6) fused directly to Ser 2 of PRMT6 and a 3′ sequence encoding a hexa His sequence (HHHHHH) (SEQ ID NO.:17) fused directly to Asp 375. The amplified gene was subcloned into pFastBacMam (Viva Biotech).

Protein Expression.

Recombinant baculovirus were generated according to Bac-to-Bac kit instructions (Life Technologies). Protein over-expression was accomplished by infecting exponentially growing HEK 293F cell culture at 1.3×10⁶ cell/ml with virus (MOI=10) in the presence of 8 mM sodium butyrate. Infections were carried out at 37° C. for 48 hours, harvested by centrifugation, and stored at −80° C. for purification.

Protein Purification.

Expressed full-length human Flag- and His-tagged PRMT6 protein was purified from cell paste by NiNTA agarose affinity chromatography after equilibration of the resin with buffer containing 50 mM Tris, 300 mM NaCl, 10% glycerol, pH 7.8 (Buffer Ni-A). Column was washed with 20 mM imidazole in the same buffer and Flag-PRMT6-His was eluted with 150 mM imidazole. Pooled fractions were dialysed against buffer Ni-A and further purified by anti-flag M2 affinity chromatography. Flag-PRMT6-His was eluted with 200 ug/ml FLAG peptide in the same buffer. Pooled fractions were dialysed in 20 mM Tris, 150 mM NaCl, 10% glycerol and 5 mM β-mercaptoethanol, pH 7.8. The purity of recovered protein was 95%.

Predicted Translations:

Flag-PRMT6-His
(SEQ ID NO.: 7)
MDYKDDDDKSQPKKRKLESGGGGEGGEGTEEEDGAEREAALERPRRTKRE
RDQLYYECYSDVSVHEEMIADRVRTDAYRLGILRNWAALRGKTVLDVGAG
TGILSIFCAQAGARRVYAVEASAIWQQAREVVRFNGLEDRVHVLPGPVET
VELPEQVDAIVSEWMGYGLLHESMLSSVLHARTKWLKEGGLLLPASAELF
IAPISDQMLEWRLGFWSQVKQHYGVDMSCLEGFATRCLMGHSEIVVQGLS
GEDVLARPQRFAQLELSRAGLEQELEAGVGGRFRCSCYGSAPMHGFAIWF
QVTFPGGESEKPLVLSTSPFHPATHWKQALLYLNEPVQVEQDTDVSGEIT
LLPSRDNPRRLRVLLRYKVGDQEEKTKDFAMEDHHHHHH

General Procedure for PRMT6 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT6, was added to columns 11, 12, 23, 24, rows I-P for the minimum signal control. A cocktail (40 ul) containing the PRMT6 enzyme was added by Multidrop Combi (Thermo-Fisher). The compounds were allowed to incubate with PRMT6 for 30 min at room temperature, then a cocktail (10 ul) containing SAM and peptide was added to initiate the reaction (final volume=51 ul). The final concentrations of the components were as follows: PRMT6 was 1 nM, ³H-SAM was 200 nM, non-radiolabeled SAM was 250 nM, peptide was 75 nM, SAH in the minimum signal control wells was 1 mM, and the DMSO concentration was 2%. The assays were stopped by the addition of non-radiolabeled SAM (10 ul) to a final concentration of 400 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

### % Inhibition Calculation

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

### Four-Parameter IC50 Fit

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT8 Biochemical Assay

General Materials.

S-adenosylmethionine (SAM), S-adenosylhomocysteine (SAH), bicine, Tween20, dimethylsulfoxide (DMSO), bovine skin gelatin (BSG), isopropyl-3-D-thiogalactopyranoside (IPTG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 31-45 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-KPAIRRLARRGGVKR-amide (SEQ ID NO.:8).

Molecular Biology:

Full-length human PRMT8 (NM_019854.4) isoform 1 transcript clone was amplified from an HEK 293 cDNA library and subcloned into pGEX-4T-1 (GE Life Sciences). The resulting construct encodes an N-terminal GST tag and a thrombin cleavage sequence (MSPILGYWKIKGLVQPTRLLLEYLEEKYEEHLYERDEGDKWRNKKFELGLEFPNLP YYIDGDVKLTQSMAIIRYIADKHNMLGGCPKERAEISMLEGAVLDIRYGVSRIAYSK DFETLKVDFLSKLPEMLKMFEDRLCHKTYLNGDHVTHPDFMLYDALDVVLYMDPM CLDAFPKLVCFKKRIEMPQIDKYLKSSKYIAWPLQGWQATFGGGDHPPKSDLVPRG SPEF) (SEQ ID NO.:9) fused directly to Met 1 of PRMT8.

Protein Expression.

E. coli (BL21(DE3) Gold, Stratagene) made competent by the CaCl₂ method were transformed with the PRMT8 construct and ampicillin selection. Protein over-expression was accomplished by growing the PRMT8 expressing E. coli clone and inducing expression with 0.3 mM IPTG at 16° C. The culture was grown for 12 hours, harvested by centrifugation, and stored at −80° C. for purification.

Protein Purification.

Expressed full-length human GST-tagged PRMT8 protein was purified from cell paste by glutathione sepharose affinity chromatography after the resin was equilibrated with 50 mM phosphate buffer, 200 mM NaCl, 5% glycerol, 5 mM β-mercaptoethanol, pH7.8 (Buffer A). GST-tagged PRMT8 was eluted with 50 mM Tris, 2 mM glutathione, pH 7.8. Pooled fractions were cleaved by thrombin (10U) and dialysed in buffer A. GST was removed by reloading the cleaved protein sample onto glutathione sepharose column and PRMT8 was collected in the flow-through fractions. PRMT8 was purified further by ceramic hydroxyapatite chromatography. The column was washed with 50 mM phosphate buffer, 100 mM NaCl, 5% glycerol, 5 mM β-mercaptoethanol, pH 7.8 and PRMT8 was eluted by 100 mM phosphate in the same buffer. Protein was concentrated and buffer was exchanged to 50 mM Tris, 300 mM NaCl, 10% glycerol, 5 mM β-mercaptoethanol, pH 7.8 by ultrafiltration. The purity of recovered protein was 89%.

Predicted Translations:

GST-tagged PRMT8
(SEQ ID NO.: 10)
MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPRGSPEFMGMKHSSRCLLLRRKM
AENAAESTEVNSPPSQPPQPVVPAKPVQCVHHVSTQPSCPGRGKMSKLL
NPEEMTSRDYYFDSYAHFGIHEEMLKDEVRTLTYRNSMYHNKHVFKDKV
VLDVGSGTGILSMFAAKAGAKKVFGIECSSISDYSEKIIKANHLDNIIT
IFKGKVEEVELPVEKVDIIISEWMGYCLFYESMLNTVIFARDKWLKPGG
LMFPDRAALYVVAIEDRQYKDFKIHWWENVYGFDMTCIRDVAMKEPLVD
IVDPKQVVTNACLIKEVDIYTVKTEELSFTSAFCLQIQRNDYVHALVTY
FNIEFTKCHKKMGFSTAPDAPYTHWKQTVFYLEDYLTVRRGEEIYGTIS
MKPNAKNVRDLDFTVDLDFKGQLCETSVSNDYKMR

General Procedure for PRMT8 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT8, was added to columns 11, 12, 23, 24, rows I-P for the minimum signal control. A cocktail (40 ul) containing the PRMT8 enzyme was added by Multidrop Combi (Thermo-Fisher). The compounds were allowed to incubate with PRMT8 for 30 min at room temperature, then a cocktail (10 ul) containing ³H-SAM and peptide was added to initiate the reaction (final volume=51 ul). The final concentrations of the components were as follows: PRMT8 was 1.5 nM, ³H-SAM was 50 nM, non-radiolabeled SAM was 550 nM, peptide was 150 nM, SAH in the minimum signal control wells was 1 mM, and the DMSO concentration was 2%. The assays were stopped by the addition of non-radiolabeled SAM (10 ul) to a final concentration of 400 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

### % Inhibition Calculation

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

### Four-Parameter IC50 Fit

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT3 Biochemical Assay

General Materials.

S-adenosylmethionine (SAM), S-adenosylhomocysteine (SAH), bicine, Tween20, dimethylsulfoxide (DMSO), bovine skin gelatin (BSG), isopropyl-β-D-thiogalactopyranoside (IPTG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide containing the classic RMT substrate motif was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-GGRGGFGGRGGFGGRGGFG-amide (SEQ ID NO.:11).

Molecular Biology:

Full-length human PRMT3 (NM_005788.3) isoform 1 transcript clone was amplified from an HEK 293 cDNA library and subcloned into pGEX-KG (GE Life Sciences). The resulting construct encodes an N-terminal GST tag and a thrombin cleavage sequence (MSPILGYWKIKGLVQPTRLLLEYLEEKYEEHLYERDEGDKWRNKKFELGLEFPNLP YYIDGDVKLTQSMAIIRYIADKHNMLGGCPKERAEISMLEGAVLDIRYGVSRIAYSK DFETLKVDFLSKLPEMLKMFEDRLCHKTYLNGDHVTHPDFMLYDALDVVLYMDPM CLDAFPKLVCFKKRIEMPQIDKYLKSSKYIAWPLQGWQATFGGGDHPPKSDLVPRG S) (SEQ ID NO.:12) fused directly to Cys 2 of PRMT3.

Protein Expression.

E. coli (BL21(DE3) Gold, Stratagene) made competent by the CaCl₂ method were transformed with the PRMT3 construct and ampicillin selection. Protein over-expression was accomplished by growing the PRMT3 expressing E. coli clone and inducing expression with 0.3 mM IPTG at 16° C. The culture was grown for 12 hours, harvested by centrifugation, and stored at −80° C. for purification.

Protein Purification.

Expressed full-length human GST-tagged PRMT3 protein was purified from cell paste by glutathione sepharose affinity chromatography after equilibration of the resin with 50 mM phosphate buffer, 200 mM NaCl, 5% glycerol, 1 mM EDTA, 5 mM β-mercaptoethanol, pH6.5 (Buffer A). GST-tagged PRMT3 was eluted with 50 mM Tris, 2 mM glutathione, pH 7.1 and 50 mM Tris, 20 mM glutathione, pH 7.1. Pooled fractions were dialysed in 20 mM Tris, 50 mM NaCl, 5% glycerol, 1 mM EDTA, 1 mM DTT, pH7.5 (Buffer B) and applied to a Q Sepharose Fast Flow column. GST-tagged PRMT3 was eluted by 500 mM NaCl in buffer B. Pooled fractions were dialyzed in 25 mM phosphate buffer, 100 mM NaCl, 5% glycerol, 2 mM DTT, pH 6.8 (Buffer C) and loaded on to a ceramic hydroxyapatite column. GST-tagged PRMT3 eluted with 25-400 mM phosphate in buffer C. Protein was concentrated and buffer was exchanged to 20 mM Tris, 150 mM NaCl, 5% glycerol, 5 mM β-mercaptoethanol, pH7.8 by ultrafiltration. The purity of recovered protein was 70%.

Predicted Translations:

GST-tagged PRMT3
(SEQ ID NO.: 13)
MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPRGSCSLASGATGGRGAVENEED
LPELSDSGDEAAWEDEDDADLPHGKQQTPCLFCNRLFTSAEETFSHCKS
EHQFNIDSMVHKHGLEFYGYIKLINFIRLKNPTVEYMNSIYNPVPWEKE
EYLKPVLEDDLLLQFDVEDLYEPVSVPFSYPNGLSENTSVVEKLKHMEA
RALSAEAALARAREDLQKMKQFAQDFVMHTDVRTCSSSTSVIADLQEDE
DGVYFSSYGHYGIHEEMLKDKIRTESYRDFIYQNPHIFKDKVVLDVGCG
TGILSMFAAKAGAKKVLGVDQSEILYQAMDIIRLNKLEDTITLIKGKIE
EVHLPVEKVDVIISEWMGYFLLFESMLDSVLYAKNKYLAKGGSVYPDIC
TISLVAVSDVNKHADRIAFWDDVYGFKMSCMKKAVIPEAVVEVLDPKTL
ISEPCGIKHIDCHTTSISDLEFSSDFTLKITRTSMCTAIAGYFDIYFEK
NCHNRVVFSTGPQSTKTHWKQTVFLLEKPFSVKAGEALKGKVTVHKNKK
DPRSLTVTLTLNNSTQTYGLQ

General Procedure for PRMT3 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT3, was added to columns 11, 12, 23, 24, rows I-P for the minimum signal control. A cocktail (40 ul) containing the PRMT3 enzyme was added by Multidrop Combi (Thermo-Fisher). The compounds were allowed to incubate with PRMT3 for 30 min at room temperature, then a cocktail (10 ul) containing SAM and peptide was added to initiate the reaction (final volume=51 ul). The final concentrations of the components were as follows: PRMT3 was 0.5 nM, ³H-SAM was 100 nM, non-radiolabeled SAM was 1.8 uM, peptide was 330 nM, SAH in the minimum signal control wells was 1 mM, and the DMSO concentration was 2%. The assays were stopped by the addition of potassium chloride (10 ul) to a final concentration of 100 mM. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm). % inhibition calculation

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

### Four-Parameter IC50 Fit

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### CARM1 Biochemical Assay

General Materials.

S-adenosylmethionine (SAM), S-adenosylhomocysteine (SAH), bicine, Tween20, dimethylsulfoxide (DMSO), bovine skin gelatin (BSG), sodium butyrate and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H3 residues 16-30 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-PRKQLATKAARKSAP-amide and contained a monomethylated arginine at position 26 (SEQ ID NO.:14).

Molecular Biology:

Human CARM1 (PRMT4) (NM_199141.1) transcript clone was amplified from an HEK 293 cDNA library, incorporating a flanking 5′ sequence encoding a FLAG tag (MDYKDDDDK) (SEQ ID NO.:6) fused directly to Ala 2 of CARM1 and 3′ sequence encoding a hexa His sequence (EGHHHHHH) (SEQ ID NO.:15) fused directly to Ser 608. The gene sequence encoding isoforml containing a deletion of amino acids 539-561 was amplified subsequently and subcloned into pFastBacMam (Viva Biotech).

Protein Expression.

Recombinant baculovirus were generated according to Bac-to-Bac kit instructions (Life Technologies). Protein over-expression was accomplished by infecting exponentially growing HEK 293F cell culture at 1.3×10⁶ cell/ml with virus (MOI=10) in the presence of 8 mM sodium butyrate. Infections were carried out at 37° C. for 48 hours, harvested by centrifugation, and stored at −80° C. for purification.

Protein Purification.

Expressed full-length human Flag- and His-tagged CARM1 protein was purified from cell paste by anti-flag M2 affinity chromatography with resin equilibrated with buffer containing 20 mM Tris, 150 mM NaCl, 5% glycerol, pH 7.8. Column was washed with 500 mM NaCl in buffer A and Flag-CARM1-His was eluted with 200 ug/ml FLAG peptide in buffer A. Pooled fractions were dialyzed in 20 mM Tris, 150 mM NaCl, 5% glycerol and 1 mM DTT, pH 7.8. The purity of recovered protein was 94.

Predicted Translations:

Flag-CARM1-His
(SEQ ID NO.: 16)
MDYKDDDDKAAAAAAVGPGAGGAGSAVPGGAGPCATVSVFPGARLLTIG
DANGEIQRHAEQQALRLEVRAGPDSAGIALYSHEDVCVFKCSVSRETEC
SRVGKQSFIITLGCNSVLIQFATPNDFCSFYNILKTCRGHTLERSVFSE
RTEESSAVQYFQFYGYLSQQQNMMQDYVRTGTYQRAILQNHTDFKDKIV
LDVGCGSGILSFFAAQAGARKIYAVEASTMAQHAEVLVKSNNLTDRIVV
IPGKVEEVSLPEQVDIIISEPMGYMLFNERMLESYLHAKKYLKPSGNMF
PTIGDVHLAPFTDEQLYMEQFTKANFWYQPSFHGVDLSALRGAAVDEYF
RQPVVDTFDIRILMAKSVKYTVNFLEAKEGDLHRIEIPFKFHMLHSGLV
HGLAFWFDVAFIGSIMTVWLSTAPTEPLTHWYQVRCLFQSPLFAKAGDT
LSGTCLLIANKRQSYDISIVAQVDQTGSKSSNLLDLKNPFFRYTGTTPS
PPPGSHYTSPSENMWNTGSTYNLSSGMAVAGMPTAYDLSSVIASGSSVG
HNNLIPLGSSGAQGSGGGSTSAHYAVNSQFTMGGPAISMASPMSIPTNT
MHYGSEGHHHHHH

General Procedure for CARM1 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of CARM1, was added to columns 11, 12, 23, 24, rows I-P for the minimum signal control. A cocktail (40 ul) containing the CARM1 enzyme was added by Multidrop Combi (Thermo-Fisher). The compounds were allowed to incubate with CARM1 for 30 min at room temperature, then a cocktail (10 ul) containing ³H-SAM and peptide was added to initiate the reaction (final volume=51 ul). The final concentrations of the components were as follows: CARM1 was 0.25 nM, ³H-SAM was 30 nM, peptide was 250 nM, SAH in the minimum signal control wells was 1 mM, and the DMSO concentration was 2%. The assays were stopped by the addition of non-radiolabeled SAM (10 ul) to a final concentration of 300 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

### % Inhibition Calculation

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

### Four-Parameter IC50 Fit

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### RKO Methylation Assay

RKO adherent cells were purchased from ATCC (American Type Culture Collection), Manassas, Va., USA. DMEM/Glutamax medium, penicillin-streptomycin, heat inactivated fetal bovine serum, 0.05% trypsin and D-PBS were purchased from Life Technologies, Grand Island, N.Y., USA. Odyssey blocking buffer, 800CW goat anti-rabbit IgG (H+L) antibody, and Licor Odyssey infrared scanner were purchased from Licor Biosciences, Lincoln, Nebr., USA. Mono-methyl arginine antibody was purchased from Cell Signaling Technology, Danvers, Mass., USA. Methanol was purchased from VWR, Franklin, Mass., USA. 10% Tween 20 was purchased from KPL, Inc., Gaithersburg, Md., USA. DRAQ5 was purchased from Biostatus Limited, Leicestershire, UK.

RKO adherent cells were maintained in growth medium (DMEM/Glutamax medium supplemented with 10% v/v heat inactivated fetal bovine serum and 100 units/mL penicillin-streptomycin) and cultured at 37° C. under 5% CO₂.

Cell treatment, In Cell Western (ICW) for detection of mono-methyl arginine and DNA content.

RKO cells were seeded in assay medium at a concentration of 20,000 cells per mL to a poly-D-lysine coated 384 well culture plate (BD Biosciences 356697) with 50 μL per well. Compound (100 nL) from a 96-well source plate was added directly to 384 well cell plate. Plates were incubated at 37° C., 5% CO₂ for 72 hours. After three days of incubation, plates were brought to room temperature outside of the incubator for ten minutes and blotted on paper towels to remove cell media. 50 μL of ice cold 100% methanol was added directly to each well and incubated for 30 min at room temperature. After 30 min, plates were transferred to a Biotek EL406 plate washer and washed 2 times with 100 μL per well of wash buffer (1×PBS). Next 60 μL per well of Odyssey blocking buffer (Odyssey Buffer with 0.1% Tween 20 (v/v)) were added to each plate and incubated 1 hour at room temperature. Blocking buffer was removed and 20 μL per well of primary antibody was added (mono-methyl arginine diluted 1:200 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:200 800CW goat anti-rabbit IgG (H+L) antibody, 1:1000 DRAQ5 (Biostatus limited) in Odyssey buffer with 0.1% Tween 20 (v/v)) and incubated for 1 hour at room temperature. The plates were washed 5 times with 100 μL per well wash buffer then 2 times with 100 μL per well of water. Plates were allowed to dry at room temperature then imaged on the Licor Odyssey machine which measures integrated intensity at 700 nm and 800 nm wavelengths. Both 700 and 800 channels were scanned.

Calculations:

First, the ratio for each well was determined by:

$$( \frac{monomethyl Arginine 800 nm value}{DRAQ 5 \; 700 nm value} )$$

Each plate included fourteen control wells of DMSO only treatment (minimum activation) as well as fourteen control wells for maximum activation treated with 20 μM of a reference compound. The average of the ratio values for each control type was calculated and used to determine the percent activation for each test well in the plate. Reference compound was serially diluted three-fold in DMSO for a total of nine test concentrations, beginning at 20 μM. Percent activation was determined and EC₃₀ curves were generated using triplicate wells per concentration of compound.

$$Percent Activation = 100 - ( ( \frac{( Individual Test Sample Ratio ) - ( Minimum Activation Ratio )}{( Maximum Activation Ratio ) - ( Minimum Activation Ratio )} ) * 100 )$$

### Other Embodiments

The foregoing has been a description of certain non-limiting embodiments of the invention. Those of ordinary skill in the art will appreciate that various changes and modifications to this description may be made without departing from the spirit or scope of the present invention, as defined in the following claims.

1. A compound that is: 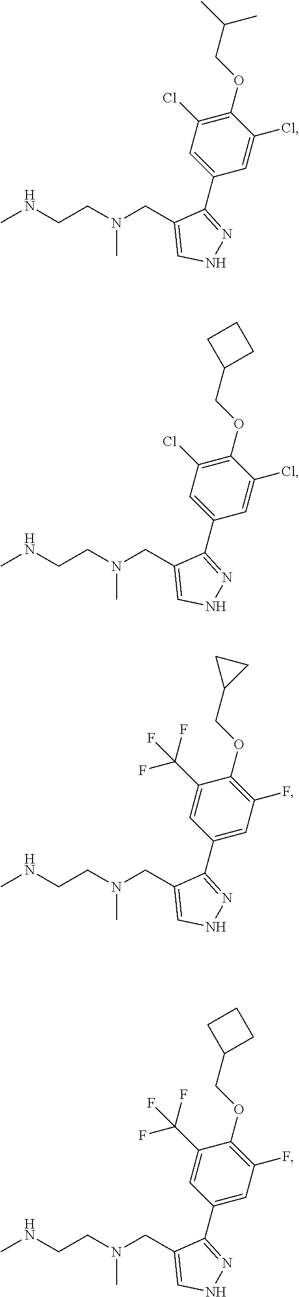 [SMILES: CNCCN(C)Cc1c[nH]nc1-c1cc(Cl)c(OCC(C)C)c(Cl)c1 ; CNCCN(C)Cc1c[nH]nc1-c1cc(Cl)c(OCC2CCC2)c(Cl)c1 ; CNCCN(C)Cc1c[nH]nc1-c1cc(F)c(OCC2CC2)c(C(F)(F)F)c1 ; CNCCN(C)Cc1c[nH]nc1-c1cc(F)c(OCC2CCC2)c(C(F)(F)F)c1] 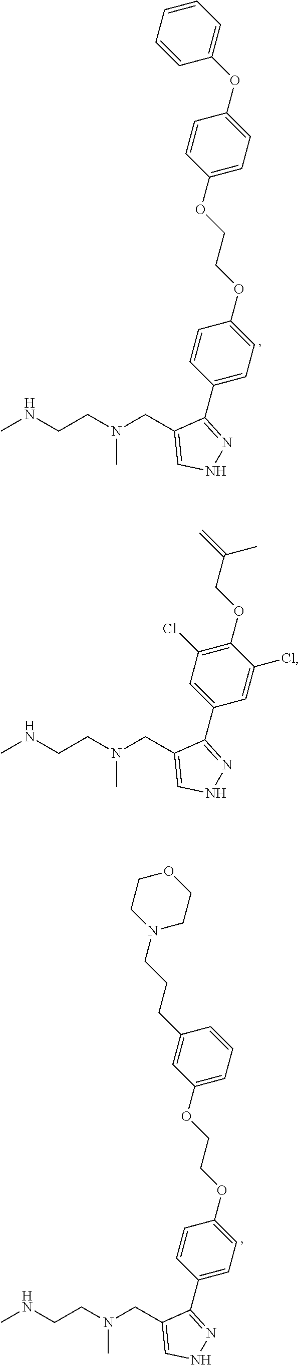 [SMILES: CNCCN(C)Cc1c[nH]nc1-c1ccc(OCCOc2ccc(Oc3ccccc3)cc2)cc1 ; "," ; C=C(C)COc1c(Cl)cc(-c2n[nH]cc2CN(C)CCNC)cc1Cl ; CNCCN(C)Cc1c[nH]nc1-c1ccc(OCCOc2cccc(CCCN3CCOCC3)c2)cc1 ; ","] 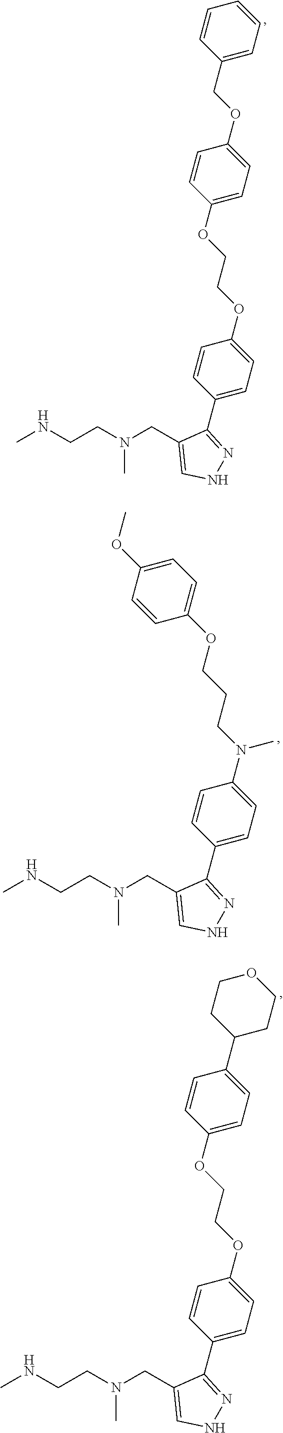 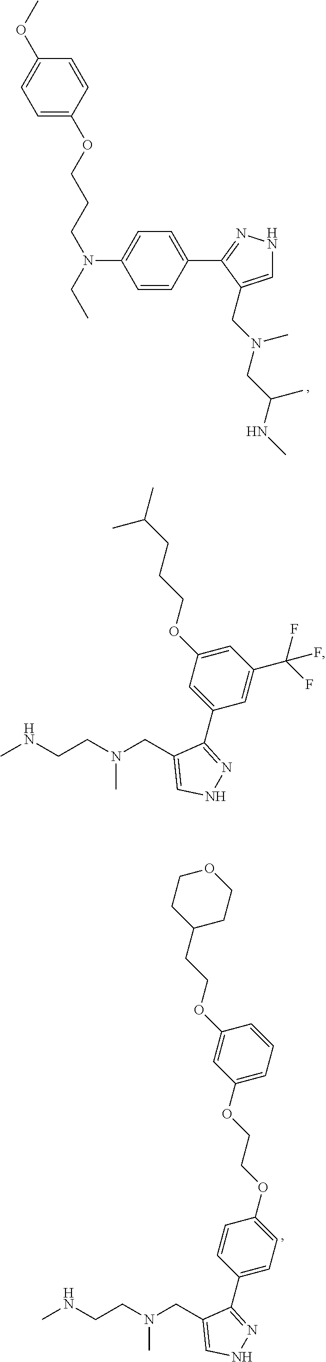 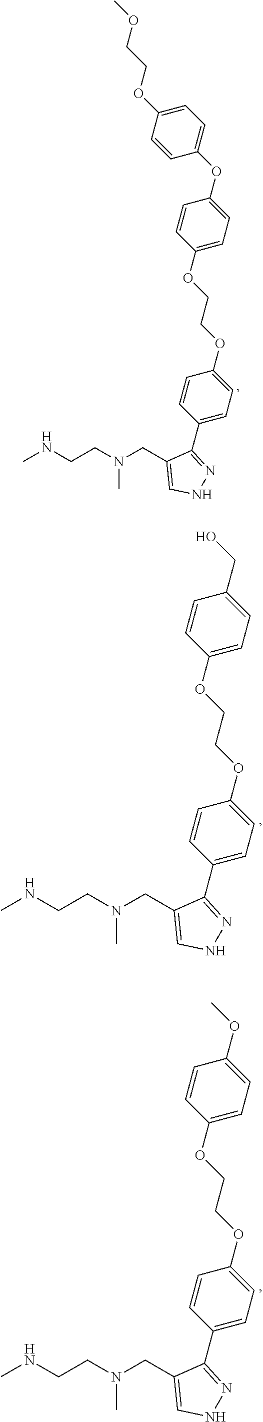 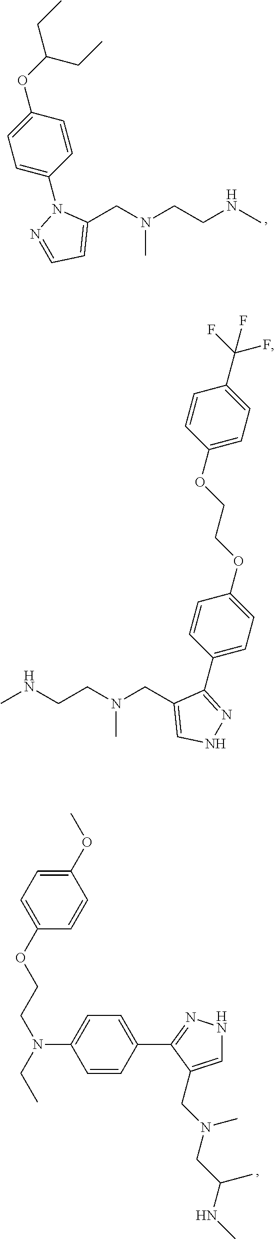 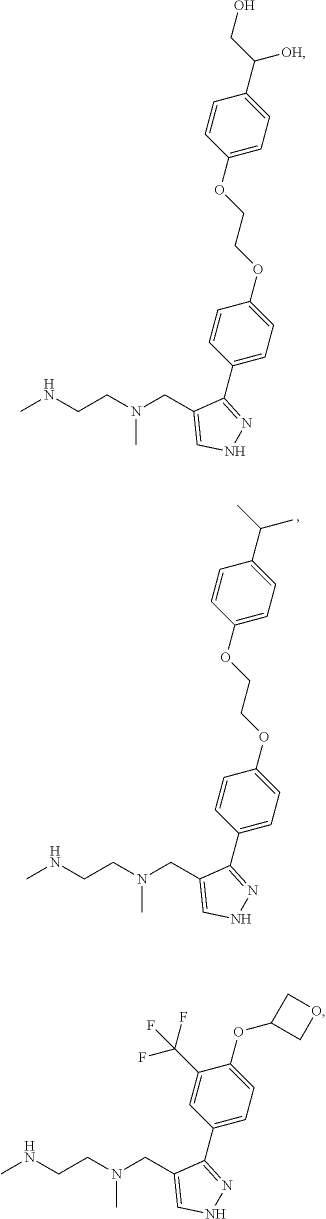 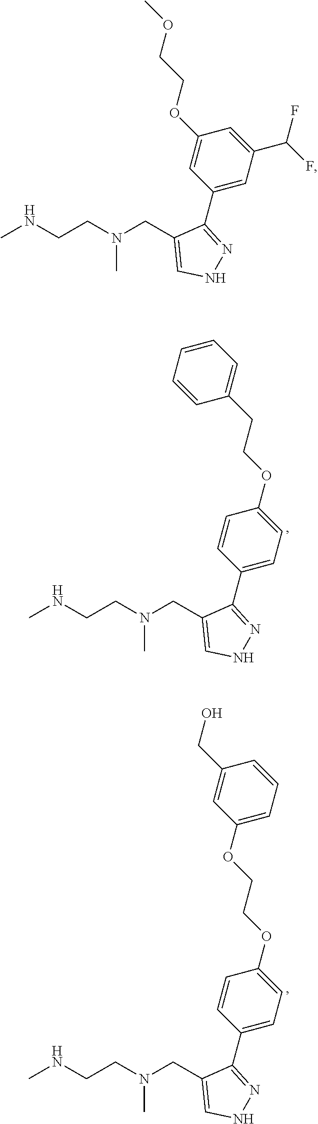 [SMILES: CNCCN(C)Cc1c[nH]nc1-c1cc(OCCOC)cc(C(F)F)c1 ; CNCCN(C)Cc1c[nH]nc1-c1ccc(OCCc2ccccc2)cc1 ; "," ; CNCCN(C)Cc1c[nH]nc1-c1ccc(OCCOc2cccc(CO)c2)cc1 ; ","] 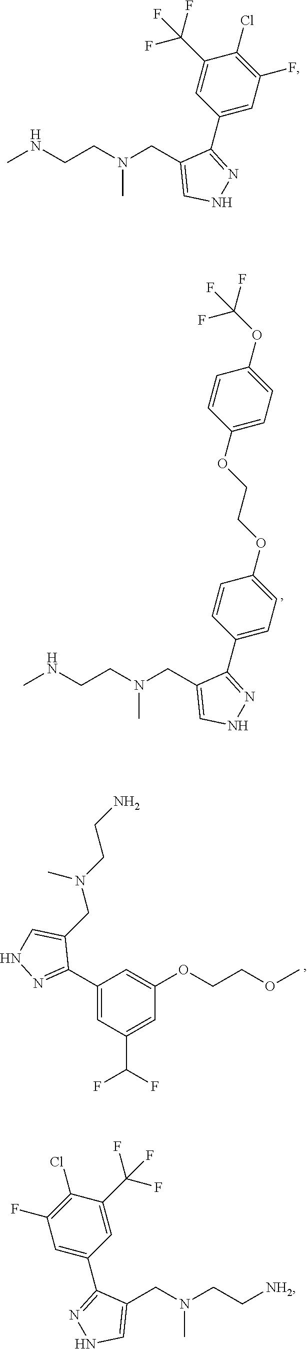 [SMILES: CNCCN(C)Cc1c[nH]nc1-c1cc(F)c(Cl)c(C(F)(F)F)c1 ; CNCCN(C)Cc1c[nH]nc1-c1ccc(OCCOc2ccc(OC(F)(F)F)cc2)cc1 ; "," ; COCCOc1cc(-c2n[nH]cc2CN(C)CCN)cc(C(F)F)c1 ; "," ; CN(CCN)Cc1c[nH]nc1-c1cc(F)c(Cl)c(C(F)(F)F)c1] 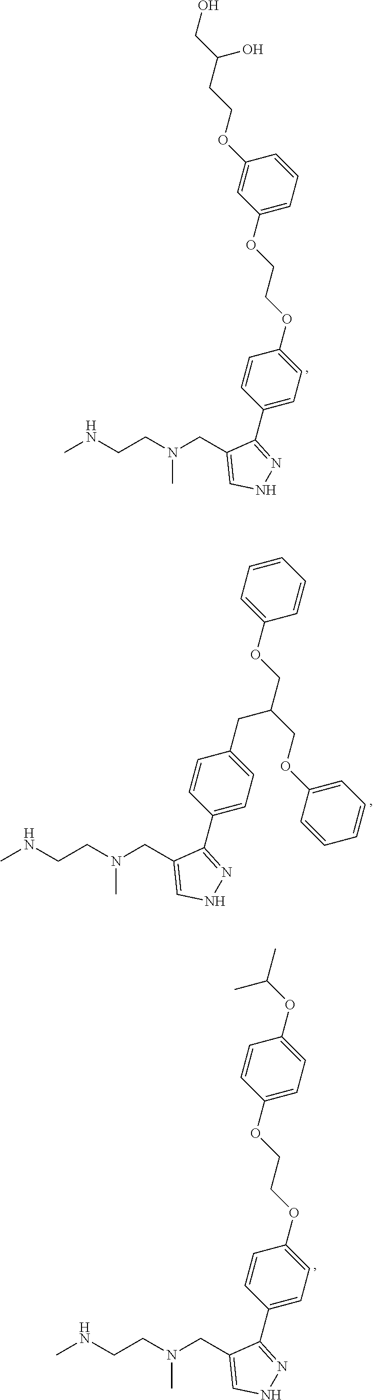 [SMILES: CNCCN(C)Cc1c[nH]nc1-c1ccc(OCCOc2cccc(OCCC(O)CO)c2)cc1 ; "," ; CNCCN(C)Cc1c[nH]nc1-c1ccc(CC(COc2ccccc2)COc2ccccc2)cc1 ; "," ; CNCCN(C)Cc1c[nH]nc1-c1ccc(OCCOc2ccc(OC(C)C)cc2)cc1 ; ","] 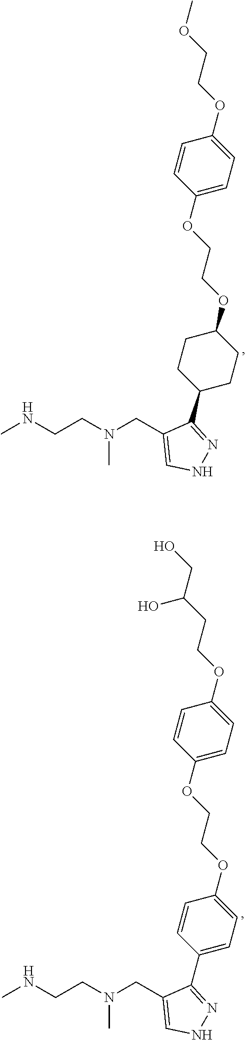 [SMILES: CNCCN(C)Cc1c[nH]nc1[C@H]1CC[C@@H](OCCOc2ccc(OCCOC)cc2)CC1 ; "," ; CNCCN(C)Cc1c[nH]nc1-c1ccc(OCCOc2ccc(OCCC(O)CO)cc2)cc1 ; ","] 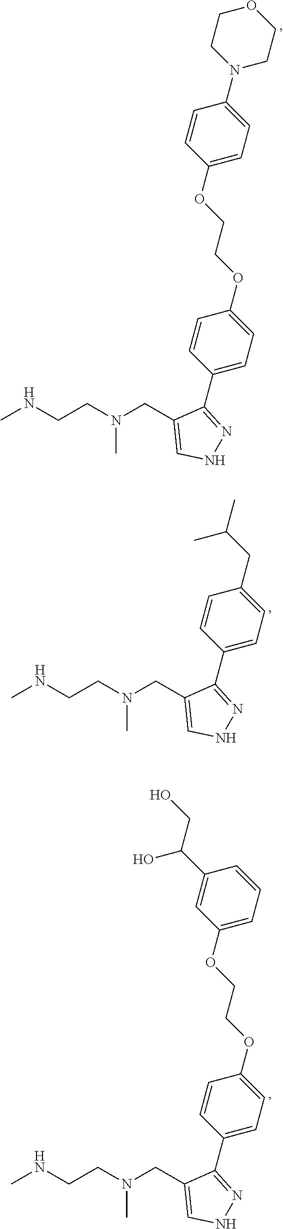 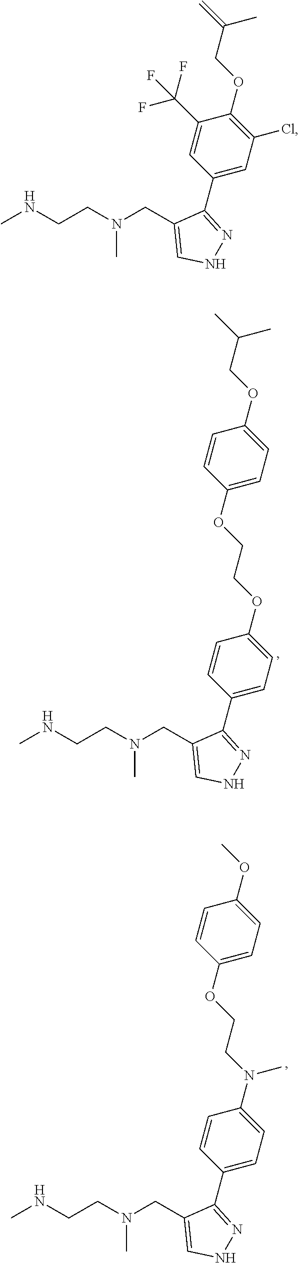 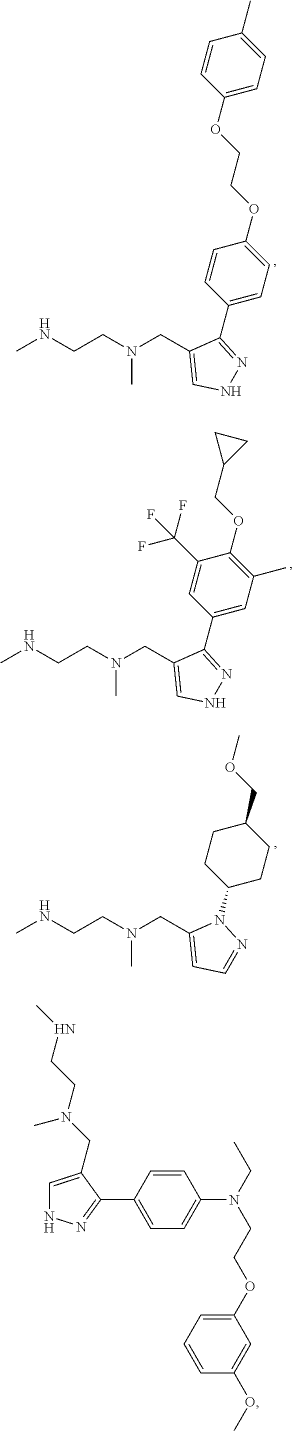 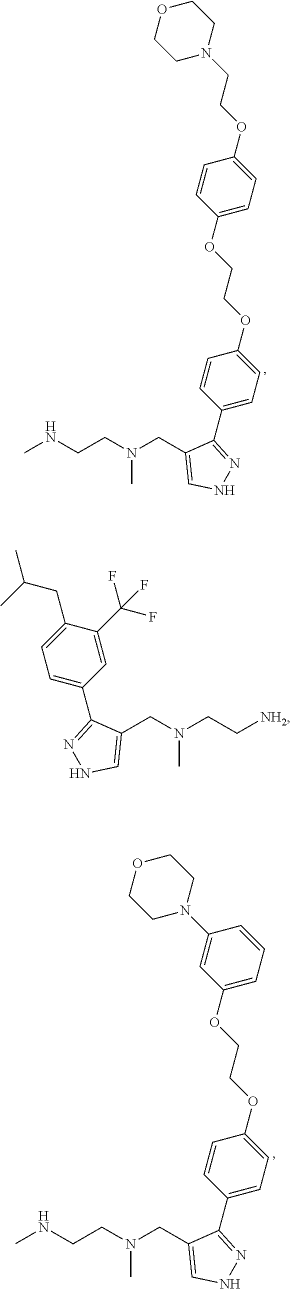 [SMILES: CNCCN(C)Cc1c[nH]nc1-c1ccc(OCCOc2ccc(OCCN3CCOCC3)cc2)cc1 ; "," ; CC(C)Cc1ccc(-c2n[nH]cc2CN(C)CCN)cc1C(F)(F)F ; CNCCN(C)Cc1c[nH]nc1-c1ccc(OCCOc2cccc(N3CCOCC3)c2)cc1 ; ","] 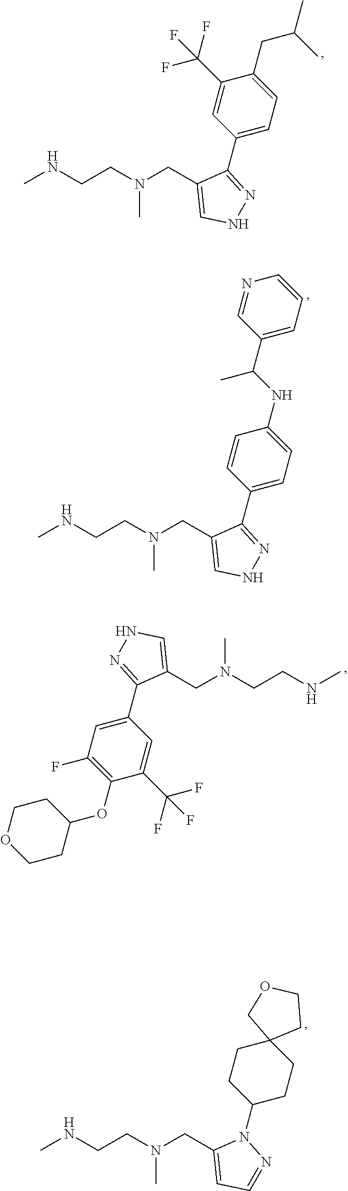 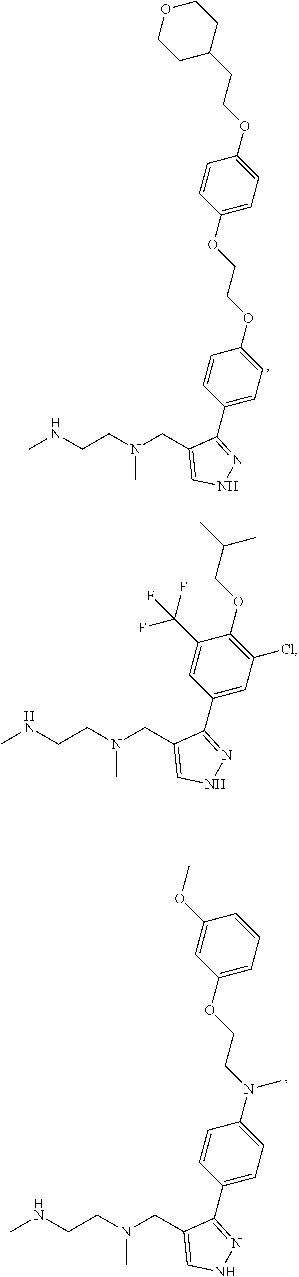 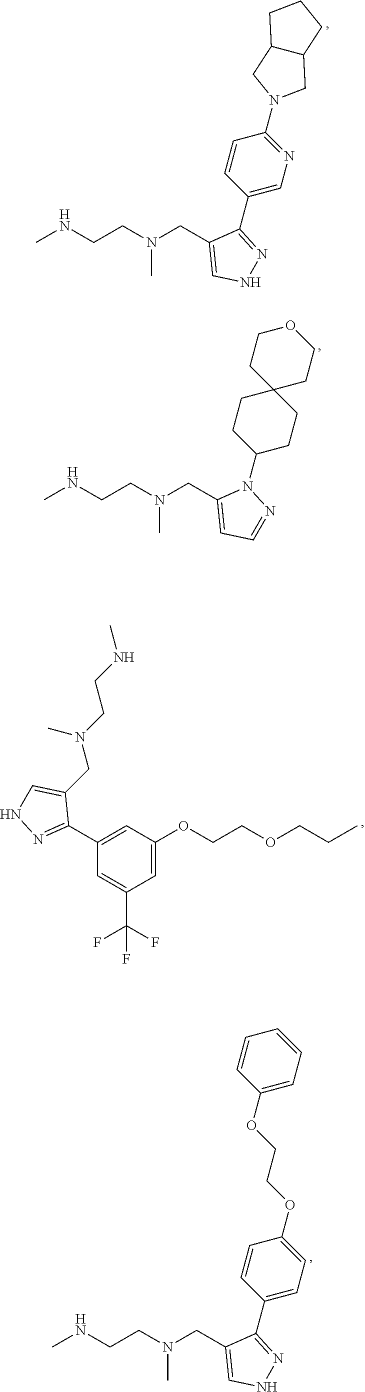 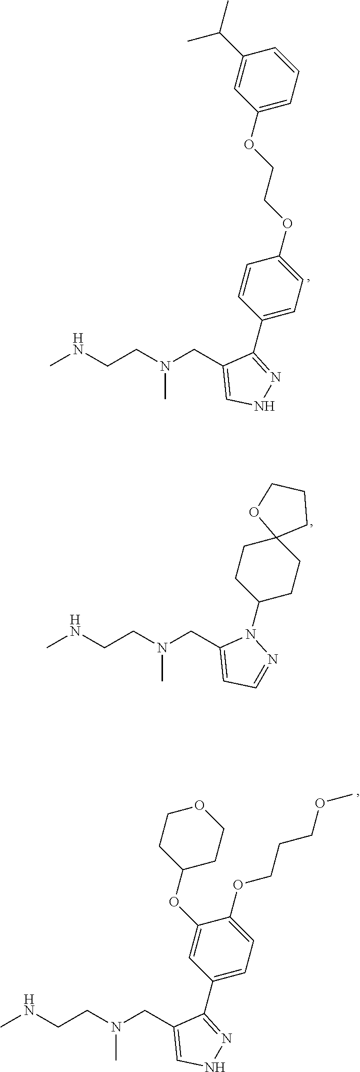 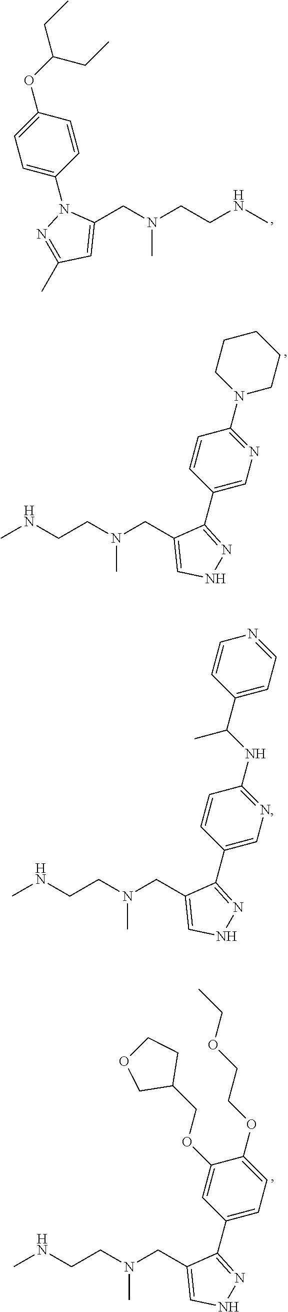 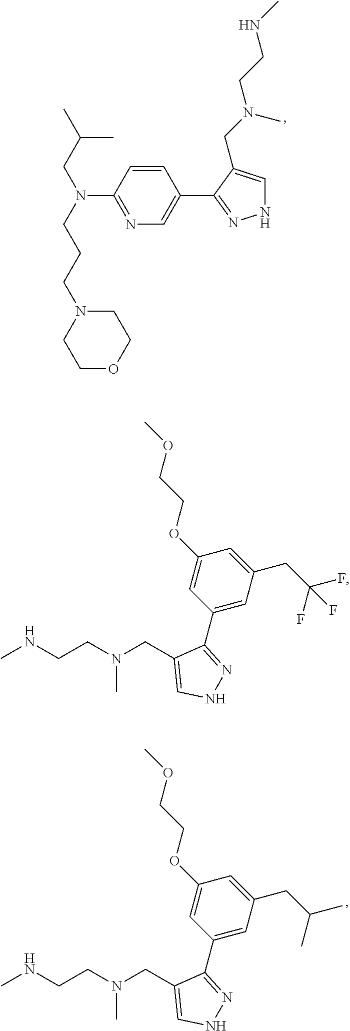 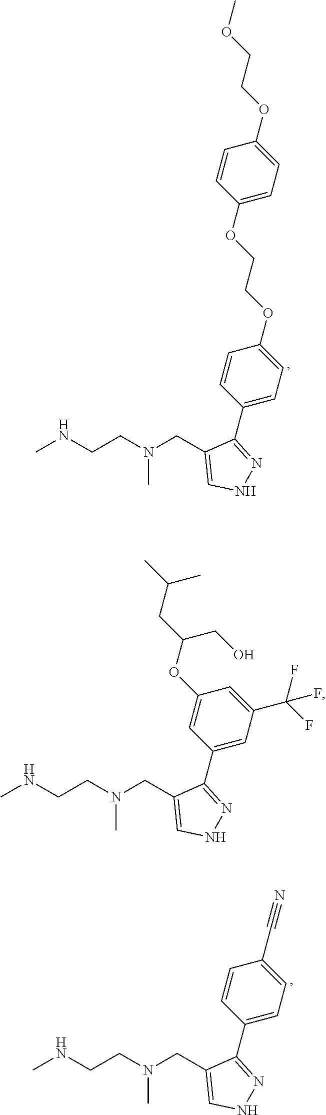 [SMILES: CNCCN(C)Cc1c[nH]nc1-c1ccc(OCCOc2ccc(OCCOC)cc2)cc1 ; "," ; CNCCN(C)Cc1c[nH]nc1-c1cc(OC(CO)CC(C)C)cc(C(F)(F)F)c1 ; CNCCN(C)Cc1c[nH]nc1-c1ccc(C#N)cc1 ; ","] 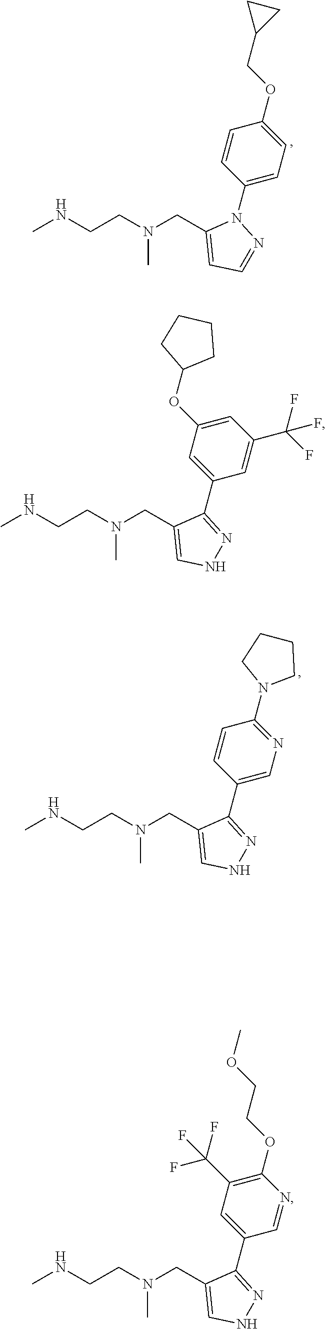 [SMILES: CNCCN(C)Cc1ccnn1-c1ccc(OCC2CC2)cc1 ; "," ; CNCCN(C)Cc1c[nH]nc1-c1cc(OC2CCCC2)cc(C(F)(F)F)c1 ; CNCCN(C)Cc1c[nH]nc1-c1ccc(N2CCCC2)nc1 ; "," ; CNCCN(C)Cc1c[nH]nc1-c1cnc(OCCOC)c(C(F)(F)F)c1] 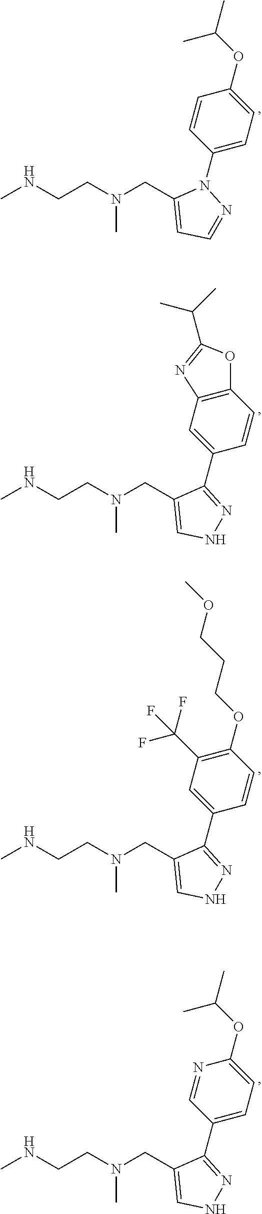 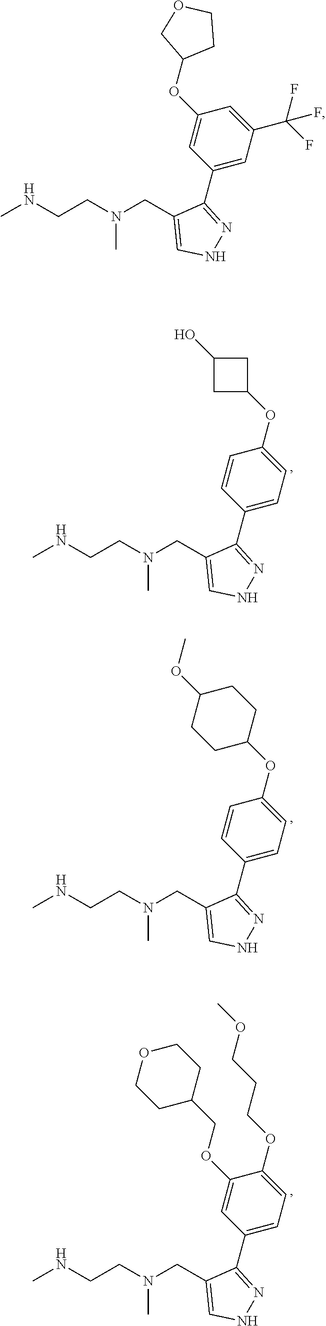 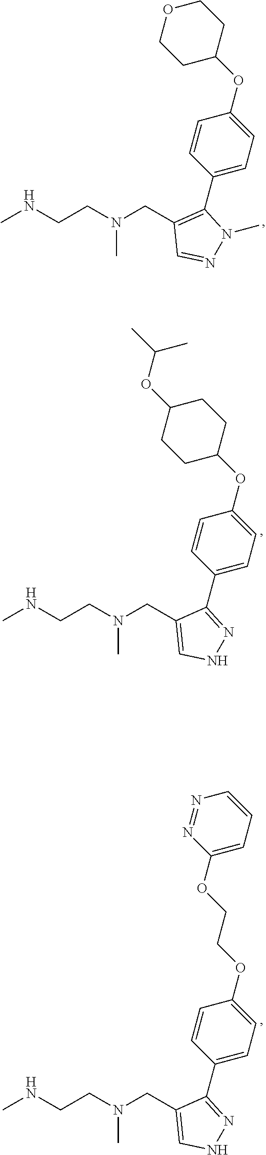 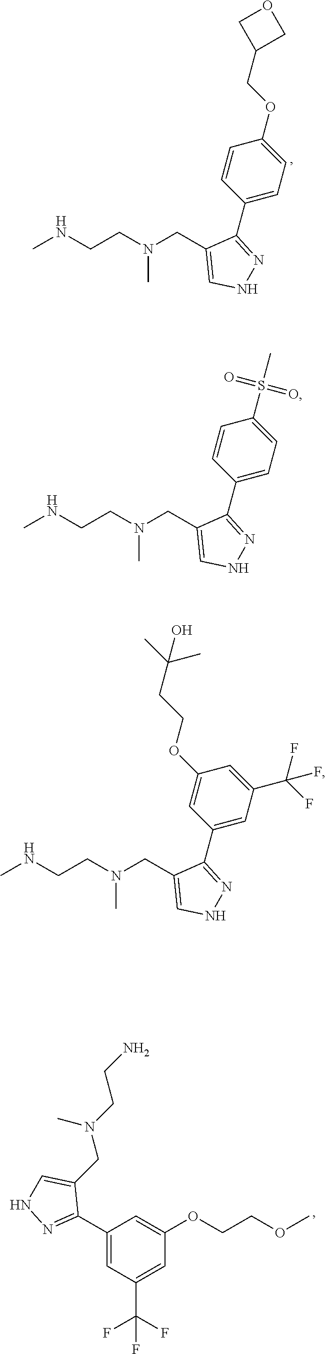 [SMILES: CNCCN(C)Cc1c[nH]nc1-c1ccc(OCC2COC2)cc1 ; "," ; CNCCN(C)Cc1c[nH]nc1-c1ccc(S(C)(=O)=O)cc1 ; CNCCN(C)Cc1c[nH]nc1-c1cc(OCCC(C)(C)O)cc(C(F)(F)F)c1 ; COCCOc1cc(-c2n[nH]cc2CN(C)CCN)cc(C(F)(F)F)c1 ; ","] 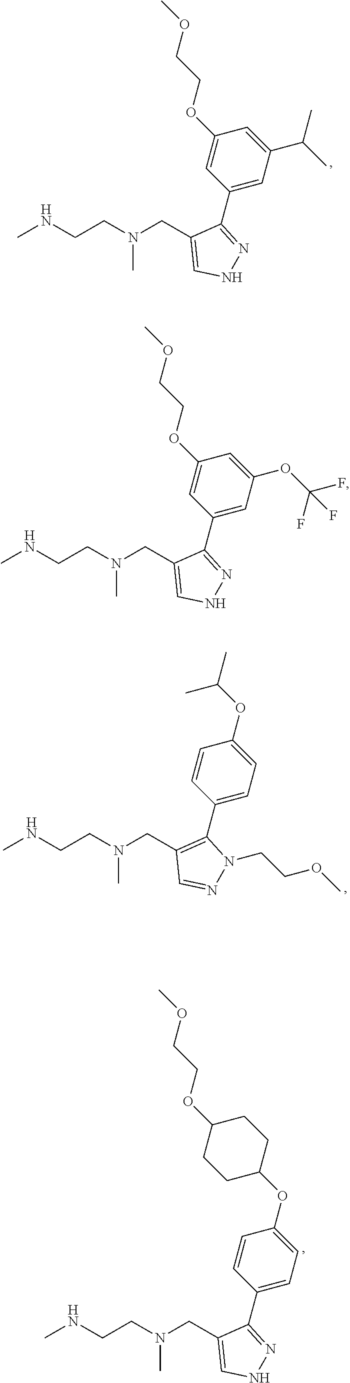 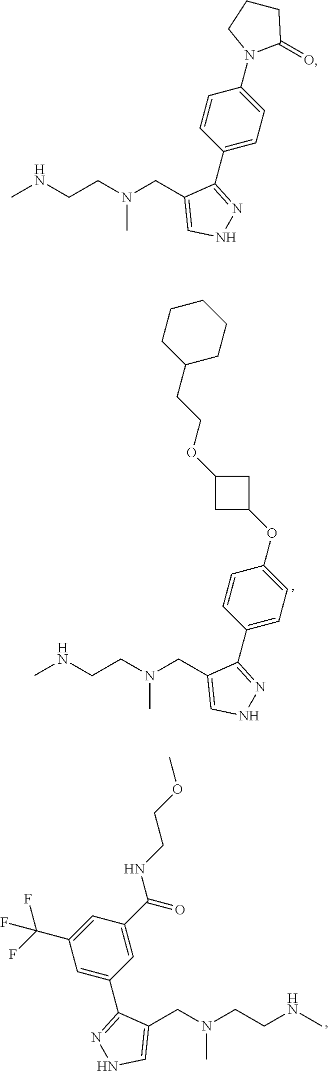 [SMILES: CNCCN(C)Cc1c[nH]nc1-c1ccc(N2CCCC2=O)cc1 ; CNCCN(C)Cc1c[nH]nc1-c1ccc(OC2CC(OCCC3CCCCC3)C2)cc1 ; "," ; CNCCN(C)Cc1c[nH]nc1-c1cc(C(=O)NCCOC)cc(C(F)(F)F)c1 ; ","] 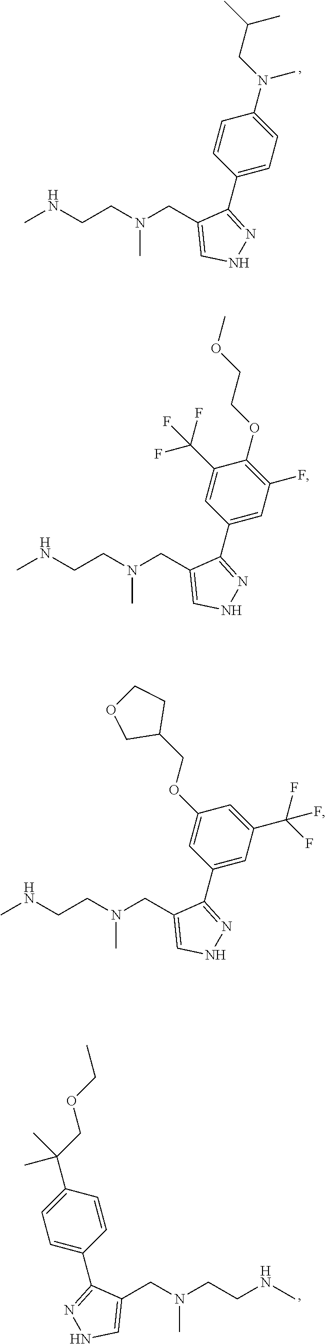 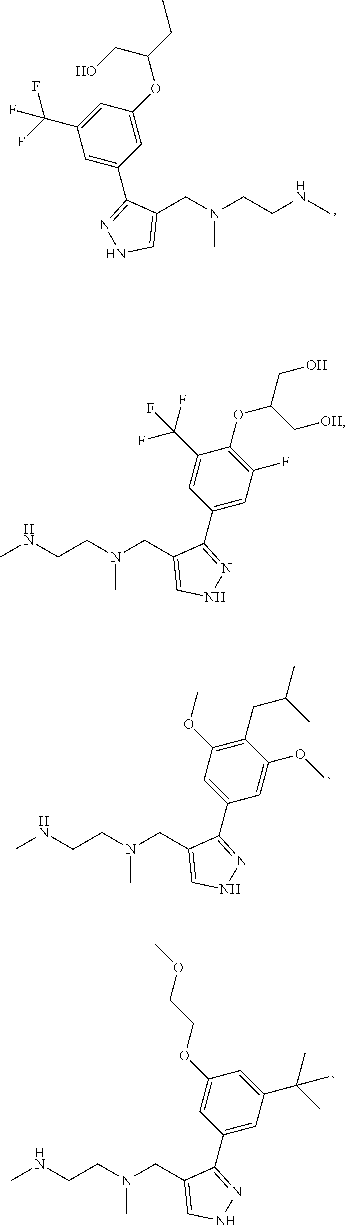 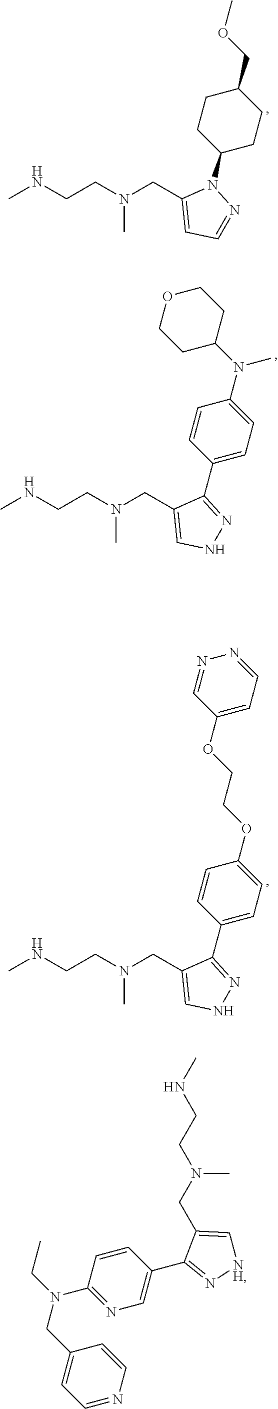 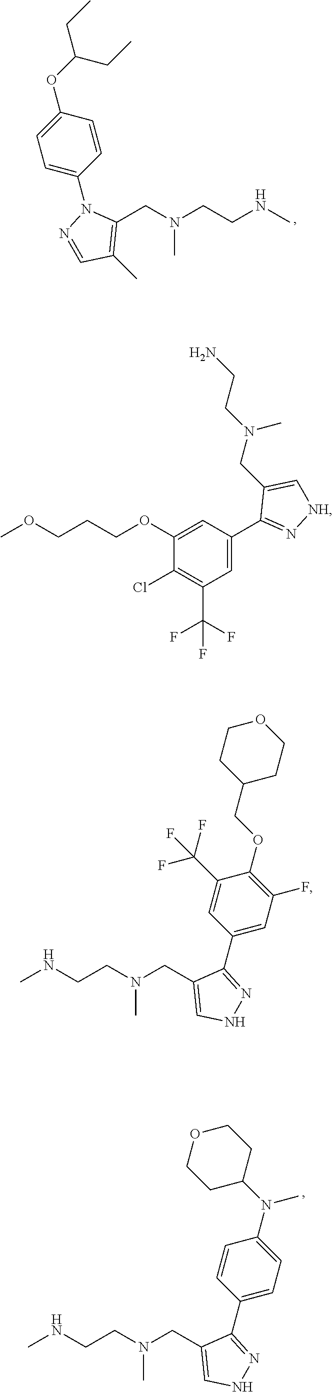 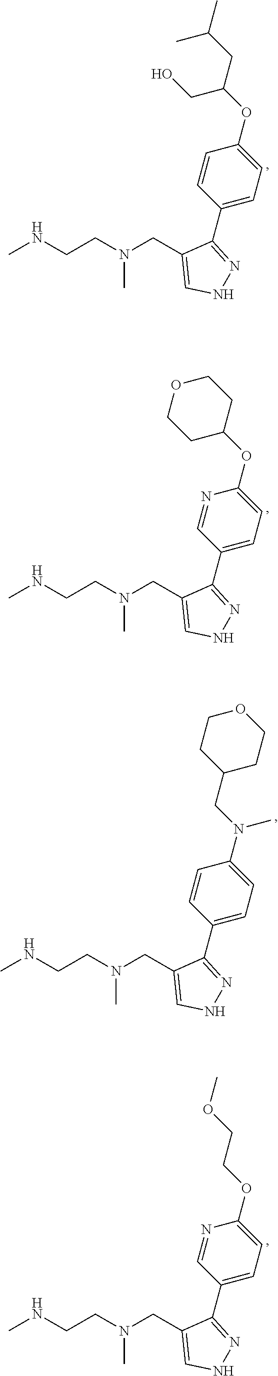 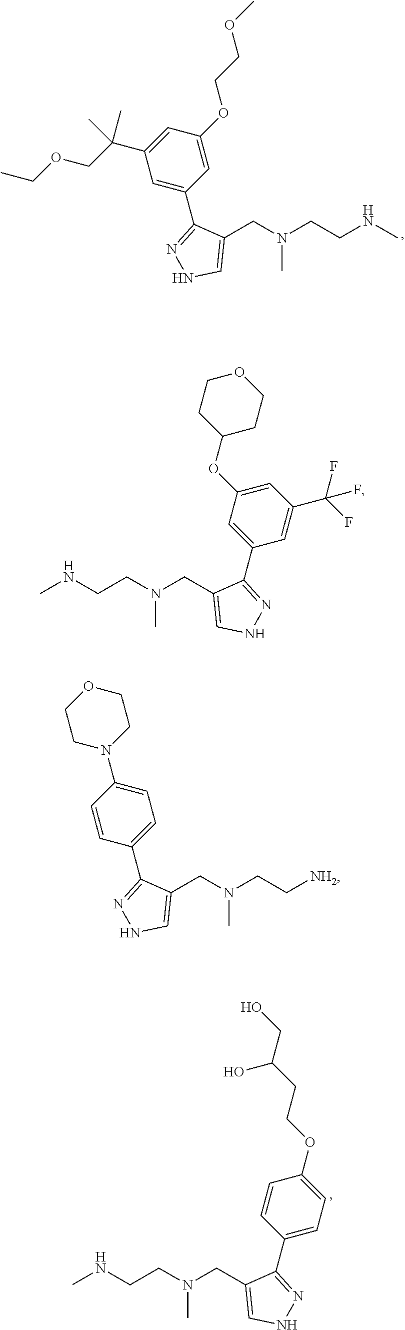 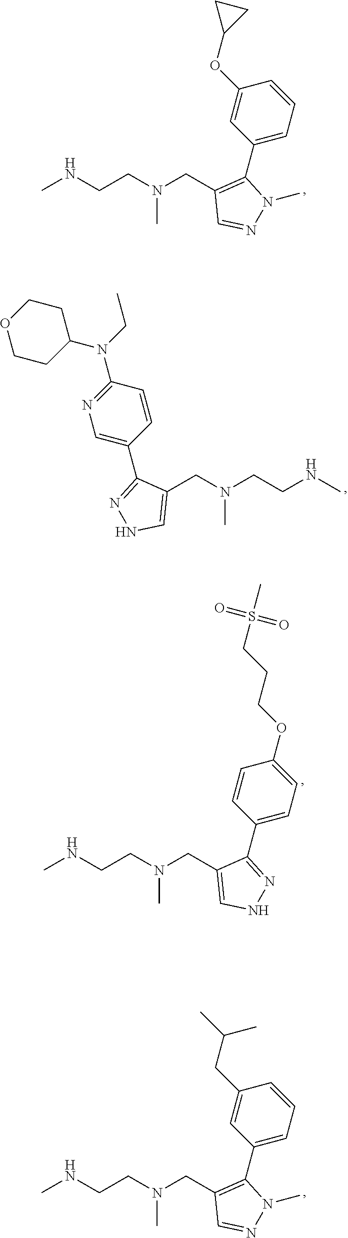 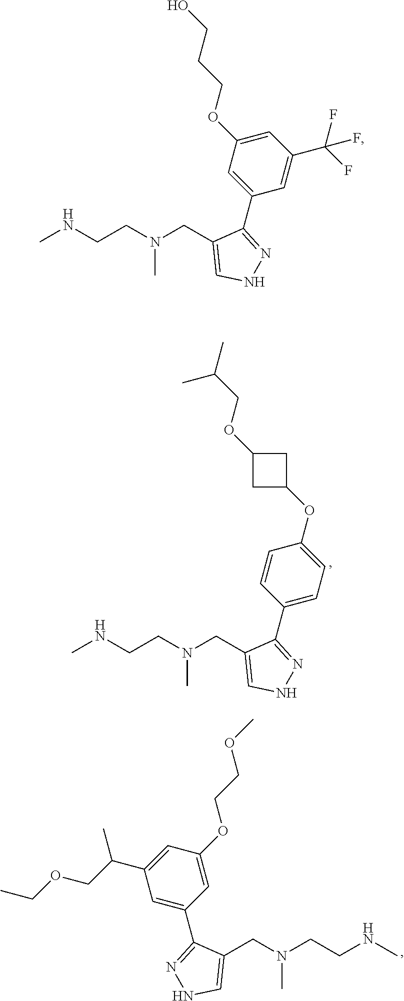 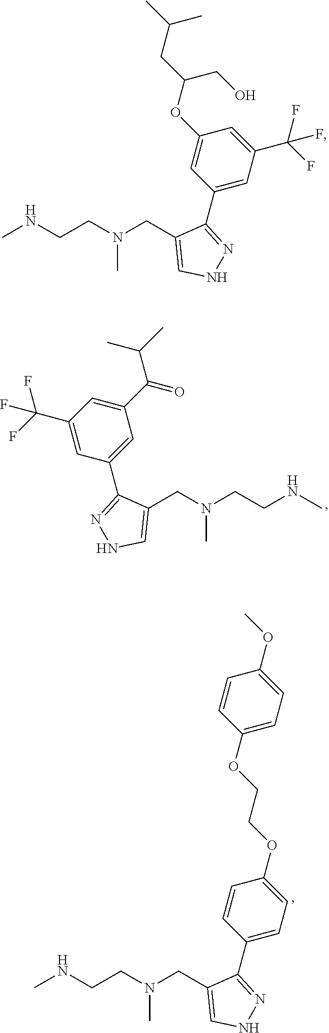 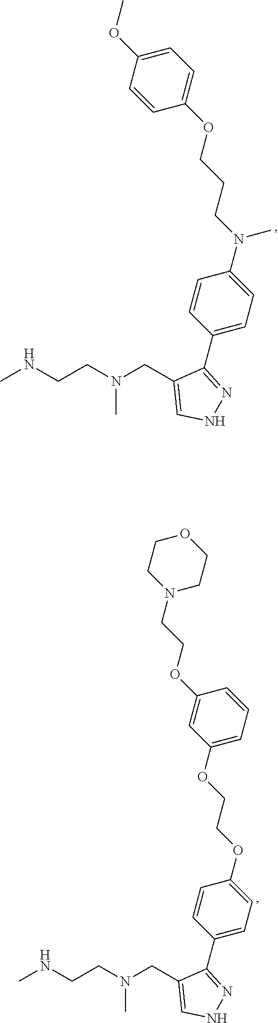 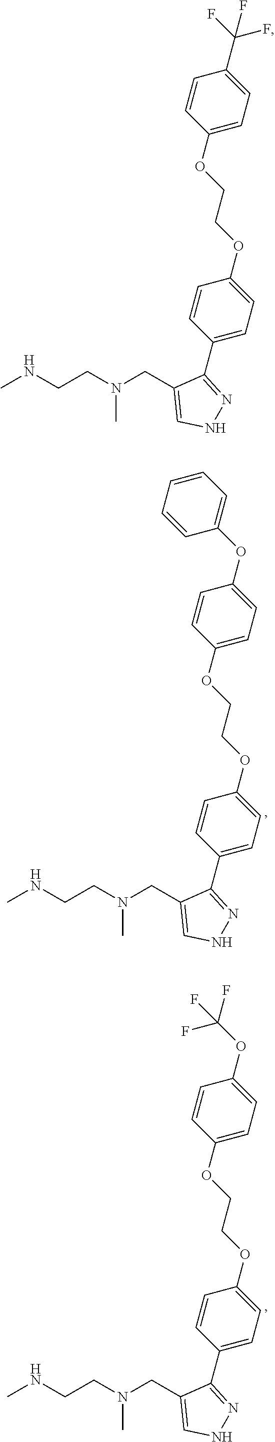 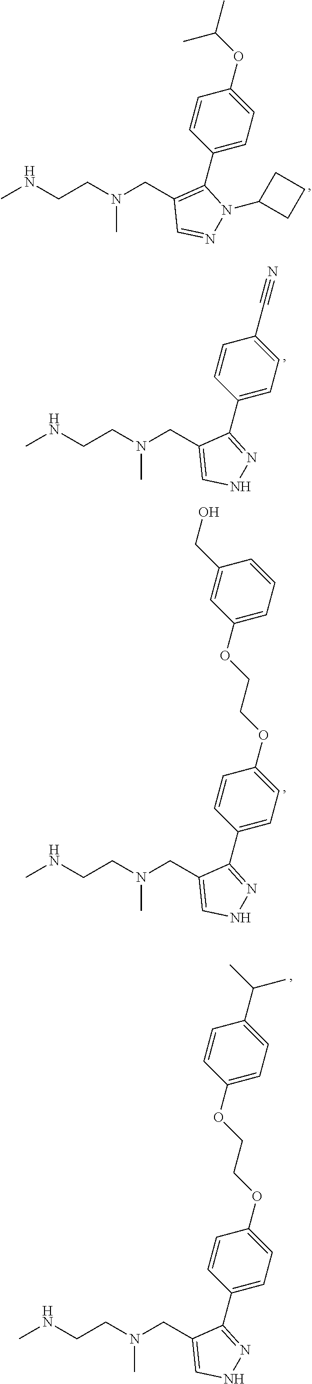 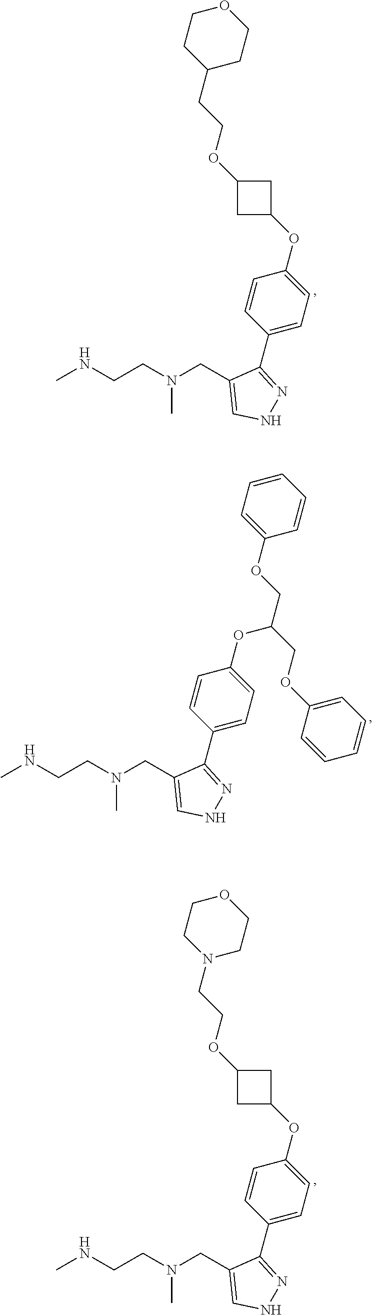 [SMILES: CNCCN(C)Cc1c[nH]nc1-c1ccc(OC2CC(OCCC3CCOCC3)C2)cc1 ; "," ; CNCCN(C)Cc1c[nH]nc1-c1ccc(OC(COc2ccccc2)COc2ccccc2)cc1 ; "," ; CNCCN(C)Cc1c[nH]nc1-c1ccc(OC2CC(OCCN3CCOCC3)C2)cc1 ; ","] 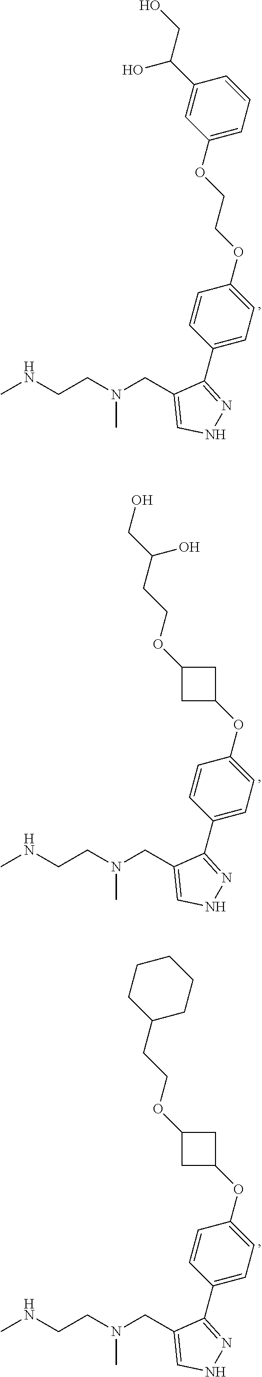 [SMILES: CNCCN(C)Cc1c[nH]nc1-c1ccc(OCCOc2cccc(C(O)CO)c2)cc1 ; "," ; CNCCN(C)Cc1c[nH]nc1-c1ccc(OC2CC(OCCC(O)CO)C2)cc1 ; "," ; CNCCN(C)Cc1c[nH]nc1-c1ccc(OC2CC(OCCC3CCCCC3)C2)cc1 ; ","] 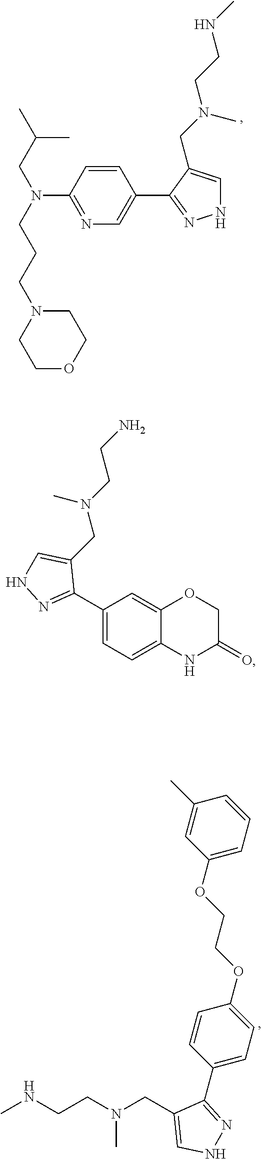 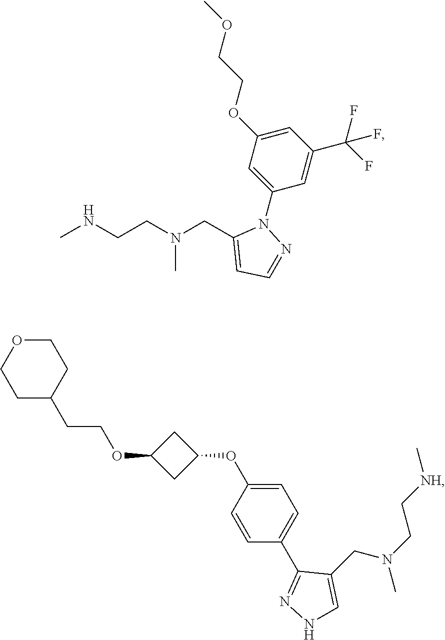 [SMILES: CNCCN(C)Cc1ccnn1-c1cc(OCCOC)cc(C(F)(F)F)c1 ; CNCCN(C)Cc1c[nH]nc1-c1ccc(O[C@H]2C[C@H](OCCC3CCOCC3)C2)cc1]
or a pharmaceutically acceptable salt thereof.

2. A pharmaceutical composition comprising a compound of claim 1 or a pharmaceutically acceptable salt thereof, and a pharmaceutically acceptable excipient.

3. A kit or packaged pharmaceutical comprising a compound of claim 1 or a pharmaceutically acceptable salt thereof, and instructions for use thereof.

4. A method of inhibiting an arginine methyl transferase (RMT) comprising contacting a cell with an effective amount of a compound of claim 1, or a pharmaceutically acceptable salt thereof.

5. The method of claim 4, wherein the arginine methyl transferase is PRMT1.

6. The method of claim 4, wherein the arginine methyl transferase is PRMT6.

7. The method of claim 4, wherein the arginine methyl transferase is PRMT8.

8. A method of modulating gene expression comprising contacting a cell with an effective amount of a compound of claim 1 or a pharmaceutically acceptable salt thereof.

9. A method of modulating transcription comprising contacting a cell with an effective amount of a compound of claim 1 or a pharmaceutically acceptable salt thereof.

10. The method of claim 4, wherein the cell is in vitro.

11. The method of claim 4, wherein the cell is in a subject.

12. A method of treating a RMT-mediated disorder, comprising administering to a subject in need thereof a therapeutically effective amount of a compound of claim 1, or a pharmaceutically acceptable salt thereof.

13. The method of claim 12, wherein the RMT-mediated disorder is a PRMT1-mediated disorder.

14. The method of claim 12, wherein the RMT-mediated disorder is a PRMT6-mediated disorder.

15. The method of claim 12, wherein the RMT-mediated disorder is a PRMT8-mediated disorder.

16. The method of claim 12, wherein the disorder is a proliferative disorder, a neurological disorder, a muscular dystrophy, an autoimmune disorder, a vascular disorder, or a metabolic disorder.

17. The method of claim 16, wherein the proliferative disorder is cancer.

18. (canceled)

19. The method of claim 16, wherein the neurological disorder is amyotrophic lateral sclerosis.

20.-23. (canceled)


SEQUENCE LISTING (17 sequences)

SEQ ID NO: 1 - protein, 15 aa, Artificial Sequence
  Synthetic Polypeptide
RLARRGGVKRISGLI

SEQ ID NO: 2 - protein, 8 aa, Artificial Sequence
  Synthetic Polypeptide
DYKDDDDK

SEQ ID NO: 3 - protein, 230 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDENLYFQGGNS

SEQ ID NO: 4 - protein, 609 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDENLYFQGGNSDYKDDDDKMAAAEAANCIMENFVATLANGMSLQPPLEEVSCGQAESSEKPNAEDMTSKDYYFDSYAHFGIHEEMLKDEVRTLTYRNSMFHNRHLFKDKVVLDVGSGTGILCMFAAKAGARKVIGIECSSISDYAVKIVKANKLDHVVTIIKGKVEEVELPVEKVDIIISEWMGYCLFYESMLNTVLYARDKWLAPDGLIFPDRATLYVTAIEDRQYKDYKIHWWENVYGFDMSCIKDVAIKEPLVDVVDPKQLVTNACLIKEVDIYTVKVEDLTFTSPFCLQVKRNDYVHALVAYFNIEFTRCHKRTGFSTSPESPYTHWKQTVFYMEDYLTVKTGEEIFGTIGMRPNAKNNRDLDFTIDLDFKGQLCELSCSTDYRMR

SEQ ID NO: 5 - protein, 15 aa, Artificial Sequence
  Synthetic Polypeptide
RLARRGGVKRISGLI

SEQ ID NO: 6 - protein, 9 aa, Artificial Sequence
  Synthetic Polypeptide
MDYKDDDDK

SEQ ID NO: 7 - protein, 389 aa, Artificial Sequence
  Synthetic Polypeptide
MDYKDDDDKSQPKKRKLESGGGGEGGEGTEEEDGAEREAALERPRRTKRERDQLYYECYSDVSVHEEMIADRVRTDAYRLGILRNWAALRGKTVLDVGAGTGILSIFCAQAGARRVYAVEASAIWQQAREVVRFNGLEDRVHVLPGPVETVELPEQVDAIVSEWMGYGLLHESMLSSVLHARTKWLKEGGLLLPASAELFIAPISDQMLEWRLGFWSQVKQHYGVDMSCLEGFATRCLMGHSEIVVQGLSGEDVLARPQRFAQLELSRAGLEQELEAGVGGRFRCSCYGSAPMHGFAIWFQVTFPGGESEKPLVLSTSPFHPATHWKQALLYLNEPVQVEQDTDVSGEITLLPSRDNPRRLRVLLRYKVGDQEEKTKDFAMEDHHHHHH

SEQ ID NO: 8 - protein, 15 aa, Artificial Sequence
  Synthetic Polypeptide
KPAIRRLARRGGVKR

SEQ ID NO: 9 - protein, 229 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEF

SEQ ID NO: 10 - protein, 623 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFMGMKHSSRCLLLRRKMAENAAESTEVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRRGEEIYGTISMKPNAKNVRDLDFTVDLDFKGQLCETSVSNDYKMR

SEQ ID NO: 11 - protein, 19 aa, Artificial Sequence
  Synthetic Polypeptide
GGRGGFGGRGGFGGRGGFG

SEQ ID NO: 12 - protein, 226 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGS

SEQ ID NO: 13 - protein, 756 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSCSLASGATGGRGAVENEEDLPELSDSGDEAAWEDEDDADLPHGKQQTPCLFCNRLFTSAEETFSHCKSEHQFNIDSMVHKHGLEFYGYIKLINFIRLKNPTVEYMNSIYNPVPWEKEEYLKPVLEDDLLLQFDVEDLYEPVSVPFSYPNGLSENTSVVEKLKHMEARALSAEAALARAREDLQKMKQFAQDFVMHTDVRTCSSSTSVIADLQEDEDGVYFSSYGHYGIHEEMLKDKIRTESYRDFIYQNPHIFKDKVVLDVGCGTGILSMFAAKAGAKKVLGVDQSEILYQAMDIIRLNKLEDTITLIKGKIEEVHLPVEKVDVIISEWMGYFLLFESMLDSVLYAKNKYLAKGGSVYPDICTISLVAVSDVNKHADRIAFWDDVYGFKMSCMKKAVIPEAVVEVLDPKTLISEPCGIKHIDCHTTSISDLEFSSDFTLKITRTSMCTAIAGYFDIYFEKNCHNRVVFSTGPQSTKTHWKQTVFLLEKPFSVKAGEALKGKVTVHKNKKDPRSLTVTLTLNNSTQTYGLQ

SEQ ID NO: 14 - protein, 15 aa, Artificial Sequence
  Synthetic Polypeptide
PRKQLATKAARKSAP

SEQ ID NO: 15 - protein, 8 aa, Artificial Sequence
  Synthetic Polypeptide
EGHHHHHH

SEQ ID NO: 16 - protein, 601 aa, Artificial Sequence
  Synthetic Polypeptide
MDYKDDDDKAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLGSSGAQGSGGGSTSAHYAVNSQFTMGGPAISMASPMSIPTNTMHYGSEGHHHHHH

SEQ ID NO: 17 - protein, 6 aa, Artificial Sequence
  Synthetic Polypeptide
HHHHHH